(19) United States
(12) Patent Application Publication
(10) Pub. No.: US 2010/0137320 A1
(43) Pub. Date: Jun. 3, 2010

(54) GAMMA SECRETASE MODULATORS

(75) Inventors: Xianhai Huang, Warren, NJ (US); Anandan Palani, Bridgewater, NJ (US); Robert G. Aslanian, Rockaway, NJ (US); Wei Zhou, Scotch Plains, NJ (US)

Correspondence Address: MERCK;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 12/393,630

(22) Filed: Feb. 26, 2009

Related U.S. Application Data
(60) Provisional application No. 61/032,545, filed on Feb. 29, 2008.

(51) Int. Cl. A61K 31/5025; C07D 487/04; A61P 25/28; A61P 25/00

(52) U.S. Cl. 514/248; 544/234

(57) ABSTRACT

This invention provides novel compounds that are modulators of gamma secretase. The compounds have the formula

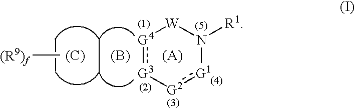

Also disclosed are methods of modulating gamma secretase activity and methods of treating Alzheimer's Disease using the compounds of formula (I).

### REFERENCE TO RELATED APPLICATION

This application claims the benefit of U.S. Provisional Application Ser. No. 61/032,545 filed Feb. 29, 2008.

### FIELD OF THE INVENTION

The present invention relates to certain heterocyclic compounds useful as gamma secretase modulators (including inhibitors, antagonists and the like), pharmaceutical compositions containing the compounds, and methods of treatment using the compounds and compositions to treat various diseases including central nervous system disorders such as, for example, neurodegenerative diseases such as Alzheimer's disease and other diseases relating to the deposition of amyloid protein. They are especially useful for reducing Amyloid beta (hereinafter referred to as Aβ) production which is effective in the treatment of diseases caused by Aβ such as, for example, Alzheimers and Down Syndrome.

### BACKGROUND OF THE INVENTION

Alzheimer's disease is a disease characterized by degeneration and loss of neurons and also by the formation of senile plaques and neurofibrillary change. Presently, treatment of Alzheimer's disease is limited to symptomatic therapies with a symptom-improving agent represented by an acetylcholinesterase inhibitor, and the basic remedy which prevents progress of the disease has not been developed. A method of controlling the cause of onset of pathologic conditions needs to be developed for creation of the basic remedy of Alzheimer's disease.

Aβ protein, which is a metabolite of amyloid precursor protein (hereinafter referred to as APP), is considered to be greatly involved in degeneration and loss of neurons as well as onset of demential conditions (for example, see Klein W L, et al Proceeding National Academy of Science USA, Sep. 2, 2003, 100(18), p. 10417-22, suggest a molecular basis for reversible memory loss.

Nitsch R M, and 16 others, Antibodies against β-amyloid slow cognitive decline in Alzheimer's disease, Neuron, May 22, 2003, 38(4), p. 547-554) suggest that the main components of Aβ protein are Aβ40 consisting of 40 amino acids and Aβ42 having two additional amino acids at the C-terminal. The Aβ40 and Aβ42 tend to aggregate (for example, see Jarrell J T et al, The carboxy terminus of the amyloid protein is critical for the seeding of amyloid formation: implications for the pathogenesis of Alzheimer's disease, Biochemistry, May 11, 1993, 32(18), p. 4693-4697) and constitute the main components of senile plaques (for example, (Glenner G G, et al, Alzheimer's disease: initial report of the purification and characterization of a novel cerebrovascular amyloid protein, Biochemical and Biophysical Research Communications, May 16, 1984, 120(3), p. 885-90. See also Masters C L, et al, Amyloid plaque core protein in Alzheimer disease and Down syndrome, Proceeding National Academy of Science USA, June 1985, 82(12), p. 4245-4249.).

Furthermore, it is known that mutations of APP and presenelin genes, which is are observed in familial Alzheimer's disease, increase production of Aβ40 and Aβ42 (for example, see Gouras G K, et al, Intraneuronal Aβ142 accumulation in human brain, American Journal of Pathology, January 2000, 156(1), p. 15-20. Also, see Scheuner D, et al, Nature Medicine, August 1996, 2(8), p. 864-870; and Forman M S, et al, Differential effects of the Swedish mutant amyloid precursor protein on β-amyloid accumulation and secretion in neurons and nonneuronal cells, Journal of Biological Chemistry, Dec. 19, 1997, 272(51), p. 32247-32253.). Therefore, compounds which reduce production of Aβ40 and Aβ42 are expected to be agents for controlling progress of Alzheimer's disease or for preventing the disease.

These Aβs are produced when APP is cleaved by beta secretase and subsequently cleaved by gamma secretase. In consideration of this, creation of inhibitors of γ-secretase and β-secretase has been attempted for the purpose of reducing production of Aβs. Many of these known secretase inhibitors are peptides or peptidomimetics such as L-685,458. L685,458, an aspartyl protease transition state mimic, is a potent inhibitor of γ-secretase activity, Biochemistry, Aug. 1, 2000, 39(30), p. 8698-8704).

Also of interest in connection with the present invention are: US 2007/0117798 (Eisai, published May 24, 2007); US 2007/0117839 (Eisai, published May 24, 2007); US 2006/0004013 (Eisai, published Jan. 5, 2006); WO 2005/110422 (Boehringer Ingelheim, published Nov. 24, 2005); WO 2006/045554 (Cellzone AG, published May 4, 2006); WO 2004/110350 (Neurogenetics™, published Dec. 23, 2004); WO 2004/071431 (Myriad Genetics, published Aug. 26, 2004); US 2005/0042284 (Myriad Genetics, published Feb. 23, 2005) and WO 2006/001877 (Myriad Genetics, published Jan. 5, 2006).

There is a need for new compounds, formulations, treatments and therapies to treat diseases and disorders associated with A. It is, therefore, an object of this invention to provide compounds useful in the treatment or prevention or amelioration of such diseases and disorders.

### SUMMARY OF THE INVENTION

In its many embodiments, the present invention provides a novel class of compounds as gamma secretase modulators (including inhibitors, antagonists and the like), methods of preparing such compounds, pharmaceutical compositions comprising one or more such compounds, methods of preparing pharmaceutical formulations comprising one or more such compounds, and methods of treatment, prevention, inhibition or amelioration of one or more diseases associated with the Aβ using such compounds or pharmaceutical compositions.

This invention provides novel compounds, that are gamma secretase modulators, of the formula:

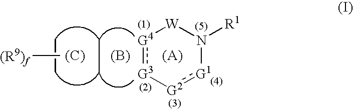

or a pharmaceutically acceptable salt, ester, or solvate thereof, wherein all substituents are defined below.

Another embodiment of this invention is directed to a compound of formula (I).

Another embodiment of this invention is directed to compound A1.

Another embodiment of this invention is directed to compound A2.

Another embodiment of this invention is directed to compound A3.

Another embodiment of this invention is directed to a pharmaceutically acceptable salt of a compound of formula (I).

Another embodiment of this invention is directed to a pharmaceutically acceptable ester of a compound of formula (I).

Another embodiment of this invention is directed to a solvate of a compound of formula (I).

Another embodiment of this invention is directed to a compound of formula (I) in isolated form.

Another embodiment of this invention is directed to a compound of formula (I) in pure form.

Another embodiment of this invention is directed to a compound of formula (I) in pure and isolated form.

Another embodiment of this invention is directed to a compound selected from the group consisting of compounds (IA) to (IH), (IJ) to (IL), 1 to 45, 1A to 45A, and 1B to 458 defined below.

Another embodiment of this invention is directed to a pharmaceutically acceptable salt of a compound selected from the group consisting of compounds (IA) to (IH), (IJ) to (IL), 1 to 45, 1A to 45A, and 18 to 45B.

Another embodiment of this invention is directed to a pharmaceutically acceptable ester of a compound selected from the group consisting of compounds (IA) to (IH), (IJ) to (IL), 1 to 45, 1A to 45A, and 1B to 45B.

Another embodiment of this invention is directed to a solvate of a compound selected from the group consisting of compounds (IA) to (IH), (IJ) to (IL), 1 to 45, 1A to 45A, and 1B to 45B.

Another embodiment is directed to a pharmaceutical composition comprising an effective amount of one or more (e.g., one) compounds of formula (I) and a pharmaceutically acceptable carrier.

Another embodiment is directed to a pharmaceutical composition comprising an effective amount of a pharmaceutically acceptable salt of one or more (e.g., one) compounds of formula (I) and a pharmaceutically acceptable carrier.

Another embodiment is directed to a pharmaceutical composition comprising an effective amount of a pharmaceutically acceptable ester of one or more (e.g., one) compounds of formula (I) and a pharmaceutically acceptable carrier.

Another embodiment is directed to a pharmaceutical composition comprising an effective amount of a solvate of one or more (e.g., one) compounds of formula (I) and a pharmaceutically acceptable carrier.

Another embodiment is directed to a pharmaceutical composition comprising an effective amount of one or more (e.g., one) compounds selected from the group consisting of compounds (IA) to (IH), (IJ) to (IL), 1 to 45, 1A to 45A, and 1B to 45B.and a pharmaceutically acceptable carrier.

Another embodiment of this invention is directed to a pharmaceutical composition comprising an effective amount of one or more (e.g., one) compounds of formula (I), and an effective amount of one or more (e.g., one) other pharmaceutically active ingredients (e.g.,) drugs, and a pharmaceutically acceptable carrier. Examples of the other pharmaceutically active ingredients include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds of formula (I), and effective amount of one or more (e.g., one) other therapeutically effective pharmaceutical active ingredients (e.g., drugs), and a pharmaceutically acceptable carrier. Examples of the other drugs include, but are not limited to drugs selected form the group consisting of: (a) drugs useful for the treatment of Alzheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase.

Another embodiment of this invention is directed to a pharmaceutical composition comprising an effective amount of one or more (e.g., one) compounds of formula (I), and effective amount of one or more BACE inhibitors, and a pharmaceutically acceptable carrier.

Another embodiment of this invention is directed to a pharmaceutical composition comprising an effective amount of one or more (e.g., one) compounds selected from the group consisting of compounds (IA) to (IH), (IJ) to (IL), 1 to 45, 1A to 45A, and 1B to 45B, and effective amount of one or more BACE inhibitors, and a pharmaceutically acceptable carrier.

Another embodiment of this invention is directed to a pharmaceutical composition comprising an effective amount of one or more (e.g., one) compounds selected from the group consisting of compounds (IA) to (IH), (IJ) to (IL), 1 to 45, 1A to 45A, and 1B to 45B, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of compounds (IA) to (IH), (IJ) to (IL), 1 to 45, 1A to 45A, and 1B to 458, and effective amount of one or more muscarinic antagonists (e.g., m₁ agonists or m₂ antagonists), and a pharmaceutically acceptable carrier.

The compounds of formula (I) can be useful as gamma secretase modulators and can be useful in the treatment and prevention of diseases such as, for example, central nervous system disorders such as Alzheimers disease and Downs Syndrome.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment.

Another embodiment of this invention is directed to a method of treating one or more neurodegenerative diseases, comprising administering an effective (i.e., therapeutically effective) amount of one or more (e.g., one) compounds of formula (I) to a patient in need of treatment.

Another embodiment of this invention is directed to a method of treating one or more neurodegenerative diseases, comprising administering an effective (i.e., therapeutically effective) amount of a compound of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment.

Another embodiment of this invention is directed to a method of treating Alzheimer's disease, comprising administering an effective (i.e., therapeutically effective) amount of one or more (e.g., one) compounds of formula (I) to a patient in need of treatment.

Another embodiment of this invention is directed to a method of treating Alzheimer's disease, comprising administering an effective (i.e., therapeutically effective) amount of a compound of formula (I) to a patient in need of treatment.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (I) and the administration of one or more (e.g., one) other pharmaceutical active ingredients (e.g., drugs). The compounds of formula (I) and the other drugs can be administered separately (i.e., each is in its own separate dosage form), or the compounds of formula (I) can be combined with the other drugs in the same dosage form.

Thus, other embodiments of this invention are directed to any one of the methods of treatment, or methods of inhibiting, described herein, wherein the compound of formula (I) is used in combination with an effective amount of one or more other pharmaceutically active ingredients selected from the group consisting of: BADE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CBI receptor inverse agonists or CBI receptor antagonists: an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Alzheimer's disease, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept®brand of donepezil hydrochloride), to a patient in need of treatment.

This invention also provides a method of treating Alzheimer's disease, comprising administering an effective (i.e., therapeutically effective) amount of one or more (e.g., one) compounds of formula (I), in combination with an effective (i.e., therapeutically effective) amount of one or more compounds selected from the group consisting of Aβ antibody inhibitors, gamma secretase inhibitors and beta secretase inhibitors.

This invention also provides a method of treating Alzheimer's disease, comprising administering an effective (i.e., therapeutically effective) amount of one or more (e.g., one) compounds of formula (I), in combination with an effective (i.e., therapeutically effective) amount of one or more BACE inhibitors.

This invention also provides a method of treating Downs syndrome, comprising administering an effective (i.e., therapeutically effective) amount of one or more (e.g., one) compounds of formula (I) to a patient in need of treatment.

This invention also provides a method of treating Downs syndrome, comprising administering an effective (i.e., therapeutically effective) amount of a compound of formula (I) to a patient in need of treatment.

This invention also provides a method of treating Downs syndrome, comprising administering an effective (i.e., therapeutically effective) amount of one or more (e.g., one) compounds of formula (I), in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

This invention also provides a method of treating Downs syndrome, comprising administering an effective (i.e., therapeutically effective) amount of a compound of formula (I), in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Other embodiments of this invention are directed to any one of the methods described above wherein the compound of formula (I) is a compound selected from the group consisting of compounds (IA) to (IH), (IJ) to (IL), 1 to 45, 1A to 45A, and 1B to 45B.

This invention also provides combinations (i.e., pharmaceutical compositions) comprising an effective (i.e., therapeutically effective) amount of one or more (e.g., one) compounds of formula (I), in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride). Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula (I) in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Compounds of formula (I) include compounds of formulas: (IA) to (IH), (IJ) to (IL), 1 to 45, 1A to 45A, and 1B to 45B.

Thus, a compound of the formula (IA) to (IH), (IJ) to (IL), 1 to 45, 1A to 45A, or 1B to 45B can be used instead of a compound of formula (I) in any one of the embodiments directed to the compounds of formula (I).

Also, a compound of the formula A1, A2, or A3 can be used instead of a compound of formula (I) in any one of the embodiments directed to the compounds of formula (I).

### DETAILED DESCRIPTION OF THE INVENTION

This invention provides compounds, useful as gamma secretase modulators, of formula (I):

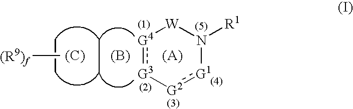

or a pharmaceutically acceptable salt, ester, or solvate thereof, wherein:

In formula (I), (A) is a reference to identify the ring;

In formula (I), (B) is a reference letter to identify the ring;

In formula (I), (C) is a reference letter to identify the ring;

Ring (B) is a 4 to 10 membered single ring or fused ring selected from the group consisting of: cycloalkyl, cycloalkenyl, aryl, heteroaryl, heterocycloalkyl, and heterocycloalkenyl, and
(a) said heterocycloalkyl Ring (B), or said heterocycloalkenyl Ring (B), or said heteroaryl Ring (B), comprises 1 to 4 heteroatoms (including any heteroatoms common to Ring (A) and Ring (C)) selected from the group consisting of —NR²—, —O—, —S—, —S(O)— and —S(O)₂, and
(b) Ring (B) is optionally substituted with 1 to 5 substitutents independently selected from the group consisting of: ═O, ═NR² and R²¹, and
(c) in one example Ring (B) is a five membered ring (including the atoms common to Rings (A) and (C)), and
(d) in another example Ring (B) is a six membered ring (including the atoms common to Rings (A) and (C));

Ring (C) is an aryl (e.g., phenyl) or heteroaryl (e.g., pyridyl) ring, and said Ring (C) is optionally substituted with 1 to 3 independently selected R²¹ substitutents;

the numbers (1), (2), (3), (4), and (5) are reference numbers to identify positions of the Ring (A);

the dotted line ( [symbol] ) between positions (1) and (2) represents an optional bond; the dotted line ( [symbol] ) between positions (3) and (4) represents an optional bond;

d is 0 or 1 (and those skilled in the art will appreciate that when d is 0 in the —N(R²)_{d}-moiety there is no substituent on the N, thus, the moiety —N(R²)_{d}— is —N═ or —NH— when d is 0, i.e., when d is 0 in a moiety there is the appropriate number of H atoms on the N to fill the required valences);

f is 1;

m is 0 to 6;

n is 1 to 5;

p is 0 to 5;

q is 0, 1 or 2, and each q is independently selected (and those skilled in the art will appreciate that when q is 0 in the moiety —C(R²¹)_{q} this means that there is no R²¹ substituent on the carbon, and the —C(R²¹)_{q} moiety is —CH═ or —CH₂—, i.e., when q is 0 in a moiety there is the appropriate number of H atoms on the carbon to fill the required valences);

W is selected from the group consisting of: —C(O)—, —S(O)₂—, —S(O)—, and —C(═NR²)—;

G¹ is selected from the group consisting of: a direct bond (i.e., the N at (5) is bonded directly to G², and Ring A is a five membered ring), —O—, —C(R²¹)_{q}, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q} group is 0 or 1 (and when 0 there is a H on the carbon), and
(b) d for the —N(R²)_{d}— group is 0 (and there is no H on the N due to the double bond between positions (3) and (4)); and
(c) G¹ is not —O—, —C(O)—, —C(═NR²)—, —S—, —S(O)₂, or S(O)—;

G² is selected from the group consisting of: a direct bond (i.e., G¹ is bonded directly to G³, and Ring A is a five membered ring), —O—, —C(R²¹)_{q}, —N(R²)_{d}—, —C(O)—, —C(═NR²)—, —S—, —S(O)₂, and S(O)—; and with the proviso that when the optional double bond between (3) and (4) is present then:
(a) q for the —C(R²¹)_{q} group is 0 or 1 (and when 0 there is a H on the carbon), and
(b) d for the —N(R²)_{d}— group is 0 (and there is no H on the N due to the double bond between positions (3) and (4)); and
(c) G² is not —O—, —C(O)—, —C(═NR²)—, —S—, —S(O)₂, or —S(O)—;

G³ is:
(a) carbon when the optional bond between G³ and G⁴ is present (i.e., there is a double bond between G³ and G⁴),
(b) carbon when the optional bond between G³ and G⁴ is absent (i.e., there is a single bond between G³ and G⁴), and there is a double bond to G³ in Ring (B), and
(c) carbon or nitrogen when the optional bond between G³ and G⁴ is absent (i.e., there is a single bond between G³ and G⁴), and there is single double bond to G³ in Ring (B);

G⁴ is:
(a) carbon when the optional bond between G³ and G⁴ is present (i.e., there is a double bond between G³ and G⁴),
(b) carbon when the optional bond between G³ and G⁴ is absent (i.e., there is a single bond between G³ and G⁴), and there is a double bond to G⁴ in Ring (B), and
(c) carbon or nitrogen when the optional bond between G³ and G⁴ is absent (i.e., there is a single bond between G³ and G⁴), and there is single double bond to G⁴ in Ring (B);

R¹ is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl (i.e., benzofusedcycloalkyl), fused benzaheterocycloalkyl (i.e., benzofusedhetero-cycloalkyl), fused heteroaryicycloalkyl (i.e., heteroarylfusedcycloalkyl), fused heteroarylheterocycloalkyl (i.e., heteroarylfusedheterocycloalkyl), heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups; or

R² is selected from the group consisting of: H, —OH, —O-alkyl (i.e., alkoxy), —O-(halo substituted alky) (such as, for example, —O-fluoroalkyl), —NH(R⁴), —N(R⁴)₂, —NH₂, —S(R⁴), —S(O)R⁴, —S(O)(OR⁴), —S(O)₂R⁴, —S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

Each R⁴ is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;

R⁹ is selected from the group consisting of: aryl-, arylalkyl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ aryl-, arylalkyl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;

R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁵), —S(O)N(R¹⁵)(R¹⁶), —S(O)₂N(R¹⁵)(R¹⁶), —C(═NOR¹⁵)R¹⁶, and —P(O)(OR¹⁵)(OR¹⁶);

R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)ₙ-alkyl, (R¹⁸)ₙ-cycloalkyl, (R¹⁸)ₙ-cycloalkylalkyl, (R¹⁵)ₙ-heterocyclyl, (R¹⁸)ₙ-heterocyclylalkyl, (R¹⁸)ₙ-aryl, (R¹⁸)ₙ-arylalkyl, (R¹⁵)ₙ-heteroaryl and (R¹⁸)ₙ-heteroarylalkyl; or

R¹⁵, R¹⁶ and R¹⁷ are independently selected from the group consisting of:

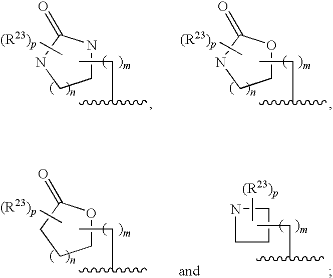

Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —ON, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or

two R¹⁸ moieties on adjacent carbons can be linked together to form a

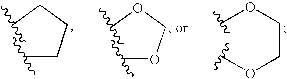

R¹⁹ is selected from the group consisting of: alkyl, cycloalkyl, aryl, arylalkyl and heteroarylalkyl;

R²⁰ is selected from the group consisting of: alkyl, cycloalkyl, aryl, halo substituted aryl, arylalkyl, heteroaryl and heteroarylalkyl;

Each R²¹ is independently selected from the group consisting of: alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR¹⁵, —C(O)R¹⁵, —C(O)OR¹⁵, —C(O)N(R¹⁵)(R¹⁶), —S(O)N(R¹⁵)(R¹⁶), —CH(R¹⁶)(R¹⁶), —S(O)₂N(R¹⁵)(R¹⁶), —C(═NOR¹⁶)R¹⁶, —P(O)(OR¹⁵)(OR¹⁶), —N(R¹⁶)(R¹⁶), -alkyl-N(R¹⁵)(R¹⁶) —N(R¹⁵)C(O)R¹⁶, —CH₂—N(R¹⁵)C(O)R¹⁶, —CH₂—N(R¹⁵)C(O)N(R¹⁶)(R¹⁶), —CH₂—R¹⁵; —CH₂N(R¹⁵)(R¹⁵), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R^{/5})C(O)OR¹⁶, —CH₂—N(R¹⁵)C(O)OR¹⁶, —S(O)R¹⁵, ═NOR¹⁵, —N₃, —NO₂, —S(O)₂R¹⁵, —O—N═C(R⁴)₂ (wherein each R⁴ is independently selected), and —O—N═C(R⁴)₂ wherein R⁴ is taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;

Each R²² group is independently selected from the group consisting of alkyl, cycloalkyl, cycloalkenyl, heterocycloalkyl, aryl, heteroaryl, halo, —CF₃, —CN, —OR¹⁵, —C(O)R¹⁶, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁶)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶—CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵;

or two R²¹ or two R²² moieties on adjacent carbons can be linked together to form a

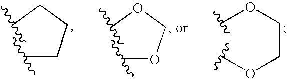

and when R²¹ or R²² is selected from the group consisting of —C(═NOR¹⁵)R¹⁶, —N(R¹⁵)C(O)R¹⁶, —CH₂—N(R¹⁶)C(O)R¹⁶, —N(R¹⁶)S(O)R¹⁶, —N(R¹⁵S(O)₂R¹⁶, —CH₂—N(R¹⁵)S(O)₂R¹⁶, —N(R¹⁵)S(O)₂N(R¹⁶)(R¹⁷), —N(R¹⁶)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁶)C(O)OR¹⁶ and —CH₂—N(R¹⁶)C(O)OR¹⁶, then R¹⁶ and R¹⁶ together can form a C₂ to C₄ chain wherein, optionally, one, two or three ring carbons can be replaced by —C(O)— or —N(H)— and R¹⁵ and R¹⁶, together with the atoms to which they are attached, form a 5 to 7 membered ring, optionally substituted by (R²³)ₙ;

Each A²³ is independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR²⁴, —C(O)R²⁴, —C(O)OR²⁴, —C(O)N(R²⁴)(R²⁶), —SR²⁴, —S(O)N(R²⁴)(R²⁶), —S(O)₂N(R²⁴)(R²⁵), —C(═NOR²⁴)R²⁶, —P(O)(OR²⁴)(OR²⁶), —N(R²⁴)(R²⁶), -alkyl-N(R²⁴)(R²⁵), —N(R²⁴)C(O)R²⁵, —CH₂—N(R²⁴)C(O)R²⁵, —N(R²⁴)S(O)R²⁵, —N(R²⁴)S(O)₂R²⁵, —CH₂—N(R²⁴)S(O)₂R²⁵, —N(R²⁴)S(O)₂N(R²⁵)(R²⁶), —N(R²⁴)S(O)N(R²⁵)(R²⁶), —N(R²⁴)C(O)N(R²⁶)(R²⁶), —CH₂—N(R²⁴)C(O)N(R²⁵)(R²⁶), —N(R²⁴)C(O)OR²⁶, —CH₂—N(R²⁴)C(O)OR²⁵, —S(O)R²⁴ and —S(O)₂R²⁴; and wherein each of the alkyl, cycloalkyl, cycloalkylalkyl, heterocycloalkyl, heterocycloalkylalkyl, aryl, arylalkyl, heteroaryl, heteroarylalkyl, alkenyl and alkynyl R²³ groups is optionally substituted with 1 to 5 independently selected R²⁷ groups;

Each R²⁴, R²⁵ and R²⁶ is independently selected from the group consisting of H, alkyl, cycloalkyl, cycloalkylalkyl, heterocycloalkyl, heterocycloalkylalkyl, aryl, arylalkyl, heteroaryl, heteroarylalkyl, arylcycloalkyl, (R^{27A})ₙ-alkyl, (R^{27A})ₙ-cycloalkyl, (R^{27A})ₙ-cycloalkylalkyl, (R^{27A})ₙ-heterocycloalkyl, (R^{27A})ₙ-heterocycloalkylalkyl, (R^{27A})ₙ-aryl, (R^{27A})ₙ-arylalkyl, (R^{27A})ₙ-heteroaryl and (R^{27A})ₙ-heteroarylalkyl;

Each R²⁷ is independently selected from the group consisting of alkyl, cycloalkyl, heterocycloalkyl, aryl, heteroaryl, halo, —CF₃, —CN, —OR²⁴, —C(O)R²⁴, —C(O)OR²⁴, alkyl-C(O)OR²⁴, C(O)N(R²⁴)(R²⁵), —SR²⁴, —S(O)N(R²⁴)(R²⁵), —S(O)₂N(R²⁴)(R²⁵), C(═NOR²⁴)R²⁵, —P(O)(OR²⁴)(OR²⁵), —N(R²⁴)(R²⁵), -alkyl-N(R²⁴)(R²⁵), —N(R²⁴)C(O)R²⁵, —CH₂—N(R²⁴)C(O)R²⁵, —N(R²⁴)S(O)R²⁵, —N(R²⁴)S(O)₂R²⁵, —CH₂—N(R²⁴)S(O)₂R²⁵, —N(R²⁴)S(O)₂N(R²⁵)(R²⁶), —N(R²⁴)S(O)N(R²⁵)(R²⁶), —N(R²⁴)C(O)N(R²⁵)(R²⁶), —CH₂—N(R²⁴)C(O)N(R²⁶)(R²⁶), —N(R²⁴)C(O)OR²⁶, —CH₂—N(R²⁴)C(O)OR²⁵, —S(O)R²⁴ and —S(O)₂R²⁴;

Each R^{27A} is independently selected from the group consisting of alkyl, aryl, arylalkyl, —NO₂, halo, —CF₃, —CN, alkyl-ON, —C(O)R²⁸, —C(O)OH, —C(O)OR²⁸, —C(O)NHR²⁹, —C(O)N(alkyl)₂, —C(O)N(alkyl)(aryl), —C(O)N(alkyl)(heteroaryl), —SR²⁸, —S(O)₂R²⁹, —S(O)NH₂, —S(O)NH(alkyl), —S(O)N(alkyl)(alkyl), —S(O)NH(aryl), —S(O)₂NH₂, —S(O)₂NHR²⁸, —S(O)₂NH(aryl), —S(O)₂NH(heterocycloalkyl), —S(O)₂N(alkyl)₂, —S(O)₂N(alkyl)(aryl), —OH, —OR²⁹, —O-heterocycloalkyl, —O-cycloalkylalkyl, —O-heterocycloalkylalkyl, —NH₂, —NHR²⁹, —N(alkyl)₂, —N(arylalkyl)₂, —N(arylalkyl)(heteroarylalkyl), —NHC(O)R²⁹, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁹, —NHS(O)₂NH(alkyl), —NHS(O)₂N(alkyl)(alkyl), —N(alkyl)S(O)₂NH(alkyl) and N(alkyl)S(O)₂N(alkyl)(alkyl);

R²⁸ is selected from the group consisting of: alkyl, cycloalkyl, arylalkyl and heteroarylalkyl; and

R²⁹ is selected from the group consisting of; alkyl, cycloalkyl, aryl, arylalkyl, heteroaryl or heteroarylalkyl; and

provided that:
(a) Ring A does not have two adjacent —O— atoms in the ring; and
(b) Ring A does not have two adjacent sulfur groups in the ring (i.e., when there is a —S—, —S(O)— or S(O)₂ group at one position in Ring A, then the adjacent positions in Ring A are not S—, —S(O)— or S(O)₂); and
(c) Ring A does not have an —O— atom adjacent to a sulfur group (i.e., Ring A does not have an —O— atom adjacent to a S—, —S(O)— or S(O)₂); and
(d) When G¹ is N, then G² is not —O—; and
(e) When G¹ is ±0-, then G² is not N; and
(f) When G¹ is N, then G² is not S—; and
(g) When G¹ is S—, then G² is not N; and
(h) When G¹ is a direct bond, and G² is —O—, then G³ is not N; and
(i) When G² is a direct bond, and —O— is —O—, then G³ is not N; and
(j) When G¹ is N, and G³ is N, then G² is not N; and
(k) When G² is N, and G³ is N, then G¹ is not N; and
(l) When G¹ is N, and G² is N, then G³ is not N.

Those skilled in the art will appreciate that when W is —S(O)—, the —S(O)— moiety can be:

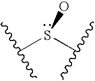

or the —S(O)— moiety can be;

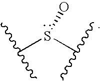

In one embodiment of this invention Ring (B) is a five membered ring (including the atoms common to Rings (A) and (C)).

In another embodiment of this invention Ring (B) is a six membered ring (including the atoms common to Rings (A) and (C)).

In another embodiment of this invention Ring (B) is cyclopentyl.

In another embodiment of this invention Ring (B) is cyclohexyl.

In another embodiment of this invention Ring (B) is cycloheptyl.

In another embodiment of this invention Ring (B) is

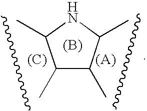

In another embodiment of this invention Ring (B) is

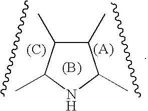

In another embodiment of this invention Ring (B) is

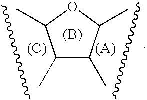

In another embodiment of this invention Ring (B) is

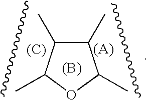

In another embodiment of this invention Ring (B) is

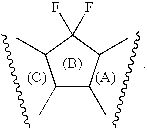

In another embodiment of this invention Ring (B) is

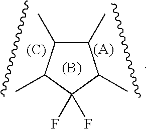

In another embodiment of this invention Ring (B) is

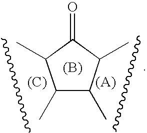

In another embodiment of this invention Ring (B) is

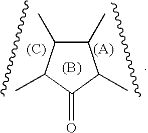

In another embodiment of this invention Ring (B) is

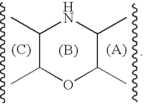

In another embodiment of this invention Ring (B) is

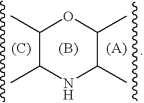

In another embodiment of this invention Ring (B) is

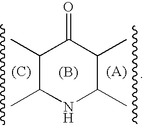

In another embodiment of this invention Ring (B) is

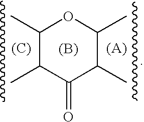

In another embodiment of this invention Ring (B) is

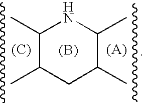

In another embodiment of this invention Ring (B) is

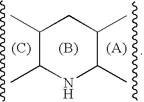

In another embodiment of this invention Ring (B) is

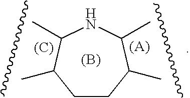

In another embodiment of this invention Ring (B) is

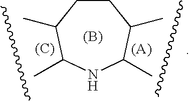

In another embodiment of this invention Ring (B) is

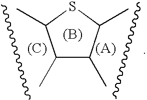

In another embodiment of this invention Ring (B) is

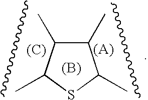

In another embodiment of this invention Ring (B) is

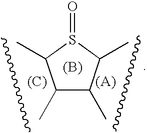

In another embodiment of this invention Ring (B) is

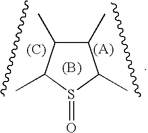

In another embodiment of this invention Ring (B) is

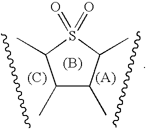

In another embodiment of this invention Ring (B) is

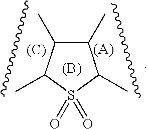

In another embodiment of this invention Ring (B) is

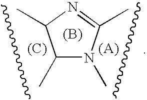

In another embodiment of this invention Ring (B) is

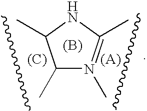

In another embodiment of this invention Ring (B) is

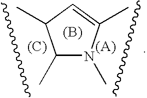

In another embodiment of this invention Ring (B) is

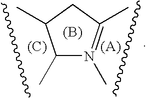

In another embodiment of this invention Ring (B) is

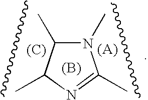

In another embodiment of this invention Ring (B) is

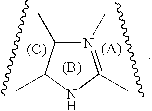

In one embodiment of this invention Ring (C) is heteroaryl (e.g., pyridyl).

In one embodiment of this invention Ring (C) is aryl.

In another embodiment of this invention Ring (C) is aryl substituted with 1 to 3 independently selected R² groups.

In another embodiment of this invention Ring (C) is aryl substituted with 1 to 3 independently selected R²¹ groups and each R²¹ group is an independently selected —OR¹⁵ group.

In another embodiment of this invention Ring (C) is aryl substituted with 1 to 3 independently selected R²¹ groups and each R²¹ group is an independently selected —OR¹⁵ group, and each R¹⁵ is an independently selected alkyl group (e.g., methyl).

In another Ring (C) is phenyl.

In another embodiment Ring (C) is phenyl substituted with 1 to 3 independently selected R²¹ groups.

In another embodiment of this invention Ring (C) is phenyl substituted with 1 to 3 independently selected R²¹ groups and each R²¹ group is an independently selected —OR¹⁵ group.

In another embodiment of this invention Ring (C) is phenyl substituted with 1 to 3 independently selected R²¹ groups and each R²¹ group is an independently selected —OR¹⁵ group, and each R¹⁵ is an independently selected alkyl group (e.g., methyl).

In another embodiment of this invention Ring (C) is phenyl substituted with 1 R²¹ group and said R²¹ group is an —OR¹⁵ group.

In another embodiment of this invention Ring (C) is phenyl substituted with 1 R²¹ group and said R²¹ group is an —OR¹⁵ group, and said R¹⁵ is an alkyl group.

In another embodiment of this invention Ring (C) is phenyl substituted with 1 R²¹ group and said R²¹ group is an —OR¹⁵ group, and said R¹⁵ is an alkyl group, and said alkyl group is methyl.

In another embodiment Ring (C) is:

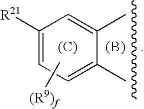

In another embodiment Ring (C) is:

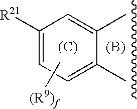

wherein R²¹ is —OR¹⁵.

In another embodiment Ring (C) is:

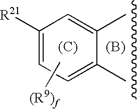

wherein R²¹ is —OR¹⁵ and R¹⁵ is alkyl.

In another embodiment Ring (C) is:

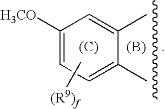

In another embodiment Ring (C) is:

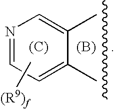

In another embodiment Ring (C) is:

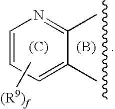

In another embodiment of this invention G¹ is a direct bond.

In another embodiment of this invention G¹ is —O—.

In another embodiment of this invention G¹ is —C(R²¹)_{q}.

In another embodiment of this invention G¹ is —N(R²)_{d}—.

In another embodiment of this invention G¹ is —C(O)—.

In another embodiment of this invention G¹ is —C(═NR²)—.

In another embodiment of this invention G is S—.

In another embodiment of this invention G¹ is —S(O)₂.

In another embodiment of this invention G¹ is —S(O)—.

In another embodiment of this invention G² is a direct bond.

In another embodiment of this invention G² is —O—.

In another embodiment of this invention G² is —C(R²¹)_{q}.

In another embodiment of this invention G² is —N(R²)_{d}—.

In another embodiment of this invention G² is —C(O)—.

In another embodiment of this invention G² is —C(═NR²)—.

In another embodiment of this invention G² is —S—.

In another embodiment of this invention G² is —S(O)₂.

In another embodiment of this invention G² is —S(O)—.

In another embodiment of this invention R²¹ is selected from the group consisting of: alkyl, —OR¹⁵, —C(O)OR¹⁵, —C(O)NR¹⁵R¹⁸, and alkyl substituted with 1 to 5 independently selected R²² groups (e.g., halo, such as, for example, F, Cl, and Br).

In another embodiment of this invention R²¹ is selected from the group consisting of: alkyl, —OR¹⁵, —C(O)OR¹⁵, —C(O)NR¹⁵R¹⁸, and alkyl substituted with 1 to 5 independently selected R²² groups (e.g., halo, such as, for example, F, Cl, and Br, and wherein in one example the alkyl substituted R²¹ group is —CF₃), wherein R¹⁵ and R¹⁶ are independently selected from the group consisting of: H, alkyl, (R¹⁸)ₙ-arylalkyl- (wherein, for example, n is 1, and R¹⁸ is —OR²⁰, and R²⁰ is alkyl (e.g., methyl), cycloalkyl (e.g., cyclobutyl), and (R¹⁸)ₙ-alkyl (e.g., n is 1, R¹⁸ is —OR²⁰, and R²⁰ is alkyl (e.g., methyl).

In another embodiment of this invention R²¹ is selected from the group consisting of: (a) alkyl, —OR¹⁵ (wherein R¹⁵ is alkyl, e.g., methyl and ethyl), (b) —C(O)OR¹⁵ (wherein R¹⁵ is alkyl, e.g., methyl), (c) —C(O)NR¹⁵R¹⁸ (wherein R¹⁵ and R¹⁸ are independently selected from the group consisting of: H, alkyl, (R¹⁸)ₙ-arylalkyl- (wherein, for example, n is 1, and R¹⁸ is ±OR²⁰, and R²⁰ is alkyl (e.g., methyl), cycloalkyl (e.g., cyclobutyl), and (R¹⁸)ₙ-alkyl (e.g., n is 1, R¹⁸ is —OR²⁰, and R²⁰ is alkyl (e.g., methyl), and in one example, only one of R¹⁵ and R¹⁶ is H), and (d) alkyl substituted with 1 to 5 independently selected R²² groups (e.g., halo, such as, for example, F, Cl, and Br, and wherein in one example the alkyl substituted R²¹ group is —CF₃).

In another embodiment of this invention R⁹ is heteroaryl.

In another embodiment of this invention R⁹ is heteroaryl substituted with one or more R²¹ groups.

In another embodiment of this invention R⁹ is heteroaryl substituted with one or more R²¹ groups, and said R²¹ groups are the same or different alkyl.

In another embodiment of this invention R⁹ is heteroaryl substituted with one R²¹ group, and said R²¹ is alkyl.

In another embodiment of this invention R⁹ is heteroaryl substituted with one R²¹ group, and said R²¹ is alkyl, and said alkyl is methyl.

In another embodiment of this invention R⁹ is and said heteroaryl is imidazoyl.

In another embodiment of this invention R⁹ is imidazolyl substituted with one or more R²¹ groups.

In another embodiment of this invention R⁹ is imidazolyl substituted with one or more R²¹ groups, and said R²¹ groups are the same or different alkyl.

In another embodiment of this invention R⁹ is imidazolyl substituted with one

R²¹ group, and said R²¹ is alkyl.

In another embodiment of this invention R⁹ is imidazolyl substituted with one or more R²¹ group, and said R²¹ is alkyl, and said alkyl is methyl.

In another embodiment of this invention Ring (B) is any one of the rings defined above, and Ring (C) is any one of the rings defined above, and said R⁹ group is selected from the group consisting of heteroaryl and heteroaryl substituted with one or more R²¹ groups, wherein each R²¹ is independently selected.

In another embodiment of this invention Ring (B) is any one of the rings defined above, and Ring (C) is any one of the rings defined above, and said R⁹ is imidazolyl substituted with one or more R²¹ groups, wherein each R²¹ is independently selected.

In another embodiment of this invention Ring (B) is any one of the rings defined above, and Ring (C) is any one of the rings defined above, and said R⁹ is imidazolyl substituted with one R²¹ group, wherein each R²¹ is independently selected.

In another embodiment of this invention Ring (B) is any one of the rings defined above, and Ring (C) is any one of the rings defined above, and said R⁹ is imidazolyl substituted with one or more independently selected alkyl groups.

In another embodiment of this invention Ring (B) is any one of the rings defined above, and Ring (C) is any one of the rings defined above, and said R⁹ is imidazolyl substituted with one alkyl group.

In another embodiment of this invention Ring (B) is any one of the rings defined above, and Ring (C) is any one of the rings defined above, and said R⁹ is imidazolyl substituted with one methyl group.

In another embodiment of this invention the R⁹ moiety is:

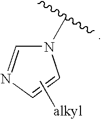

In another embodiment of this invention the R⁹—R(C)— moiety is:

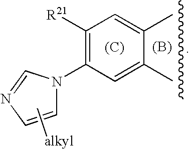

In another embodiment of this invention the R⁹—R(C)— moiety is:

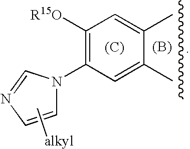

And in another embodiment said R¹⁵ is alkyl in said R⁹—R(C)— moiety.

In another embodiment of this invention the R⁹—R(C)— moiety is:

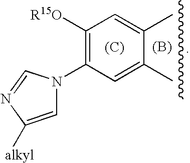

And in another embodiment said R^{th} is alkyl in said R⁹—R(C)— moiety.

In another embodiment of this invention the R⁹—R(C)— moiety is:

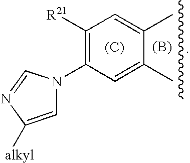

In another embodiment of this invention the R⁹—R(C)— moiety is:

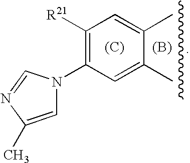

In another embodiment of this invention the R⁹—R(C)— moiety is:

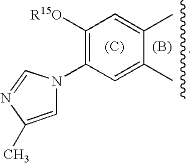

And in another embodiment said R¹⁵ is alkyl in said R⁹—R(C)— moiety.

In another embodiment of this invention the R⁹—R(C)— moiety is:

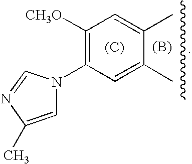

In another embodiment of this invention the R⁹—R(C)— moiety is:

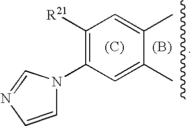

In another embodiment of this invention the R⁹—R(C)— moiety is:

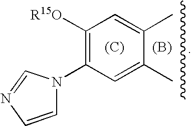

In another embodiment of this invention the R⁹—R(C)— moiety is:

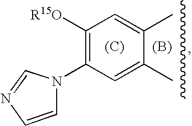

wherein said R¹⁵ is alkyl.

In another embodiment of this invention the R⁹—R(C)— moiety is:

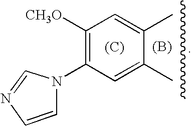

In another embodiment of this invention R¹ is an alkyl group substituted with one or more independently selected R²¹ groups.

In another embodiment of this invention R¹ is:

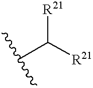

wherein each R²¹ is independently selected, and each R²¹ is independently unsubstituted or substituted with one or more independently selected R²² groups.

In another embodiment of this invention R¹ is:

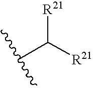

wherein one R²¹ is an unsubstituted or substituted alkyl group.

In another embodiment of this invention R¹ is:

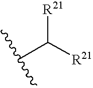

wherein one R²¹ is an unsubstituted alkyl group.

In another embodiment of this invention R¹ is:

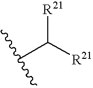

wherein one R²¹ is a substituted alkyl group.

In another embodiment of this invention R¹ is:

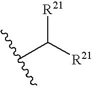

wherein one R²¹ is an unsubstituted or substituted alkyl group, and the other R²¹ is an unsubstituted or substituted aryl (e.g., phenyl) group.

In another embodiment of this invention R¹ is:

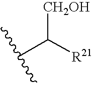

and R²¹ is unsubstituted or substituted with one or more independently selected R²² groups.

In another embodiment of this invention R¹ is:

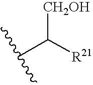

and R²¹ is unsubstituted aryl (e.g., phenyl) or aryl (e.g., phenyl) substituted with one or more independently selected R²² groups.

In another embodiment of this invention R¹ is an alkyl group substituted with one R²¹ group.

In another embodiment of this invention R¹ is an alkyl group substituted with one R²¹ group, and said R²¹ group is substituted with one or more independently selected R²² groups.

In another embodiment of this invention R¹ is:

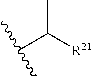

wherein R²¹ is unsubstituted or substituted with one or more independently selected R²² groups.

In another embodiment of this invention R¹ is an alkyl group substituted with one R²¹ group, and said R²¹ group is an aryl group.

in another embodiment of this invention R¹ is an alkyl group substituted with one R²¹ group, and said R²¹ group is an aryl group, said aryl is phenyl.

in another embodiment of this invention R¹ is an ethyl group substituted with one R²¹ group, and said R²¹ group is an aryl group, said aryl is phenyl.

In another embodiment of this invention R¹ is an methyl group substituted with one R²¹ group, and said R²¹ group is an aryl group, said aryl is phenyl.

In another embodiment of this invention R¹ is an alkyl group substituted with one R²¹ group, and said R²¹ group is an aryl group, and said aryl group is substituted with one or more R²² groups.

In another embodiment of this invention R¹ is an alkyl group substituted with one R²¹ group, and said R²¹ group is an aryl group, and said aryl group is substituted with one or more R²² groups, and each R²² group is the same or different halo.

In another embodiment of this invention R¹ is an alkyl group substituted with one R²¹ group, and said R²¹ group is an aryl group, and said aryl group is substituted with 1 to 3 R²² groups, and each R²² group is the same or different halo.

In another embodiment of this invention R¹ is an alkyl group substituted with one R²¹ group, and said R²¹ group is an aryl group, and said aryl group is substituted with one or two R²² halo groups, and each R²² group is the same or different halo.

In another embodiment of this invention R¹ is an alkyl group substituted with one R²¹ group, and said R²¹ group is an aryl group, and said aryl group is substituted with one or two R²² F groups.

In another embodiment of this invention R¹ is an alkyl group substituted with one R²¹ group, and said R²¹ group is phenyl, and said phenyl is substituted with one or more R²² groups.

In another embodiment of this invention R¹ is an alkyl group substituted with one R²¹ group, and said R²¹ group is phenyl, and said phenyl is substituted with one or more R²² groups, and each R²² group is the same or different halo.

In another embodiment of this invention R¹ is an alkyl group substituted with one R²¹ group, and said R²¹ group is phenyl, and said phenyl is substituted with one or two R²² halo groups, and each R²² group is the same or different halo.

In another embodiment of this invention R¹ is an alkyl group substituted with one R²¹ group, and said R²¹ group is phenyl, and said phenyl is substituted with one or two R²² F groups.

In another embodiment of this invention R¹ is an ethyl group substituted with one R²¹ group, and said R²¹ group is an aryl group, and said aryl group is substituted with one or more R²² groups.

In another embodiment of this invention R¹ is a methyl group substituted with one R²¹ group, and said R²¹ group is an aryl group, and said aryl group is substituted with one or more R²² groups.

In another embodiment of this invention R¹ is an ethyl group substituted with one R²¹ group, and said R²¹ group is phenyl, and said phenyl is substituted with one or more R²² groups.

In another embodiment of this invention R¹ is a methyl group substituted with one R²¹ group, and said R²¹ group is phenyl, and said phenyl is substituted with one or more R²² groups.

In another embodiment of this invention R¹ is an ethyl group substituted with one R²¹ group, and said R²¹ group is phenyl, and said phenyl is substituted with one or two R²² halo groups, and each R²² group is the same or different halo.

In another embodiment of this invention R¹ is a methyl group substituted with one R²¹ group, and said R²¹ group is phenyl, and said phenyl is substituted with one or two R²² halo groups, and each R²² group is the same or different halo.

In another embodiment of this invention R¹ is an ethyl group substituted with one R²¹ group, and said R²¹ group is phenyl, and said phenyl is substituted with one or two R²² F groups.

In another embodiment of this invention R¹ is a methyl group substituted with one R²¹ group, and said R²¹ group is phenyl, and said phenyl is substituted with one or two R²² F groups.

In another embodiment of this invention R¹ is an ethyl group substituted with one R²¹ group, and said R²¹ group is phenyl, and said phenyl is substituted with one R²² halo group.

In another embodiment of this invention R¹ is a methyl group substituted with one R²¹ group, and said R²¹ group is phenyl, and said phenyl is substituted with one R²² halo group.

In another embodiment of this invention R¹ is an ethyl group substituted with one R²¹ group, and said R²¹ group is phenyl, and said phenyl is substituted with one R²² F group.

In another embodiment of this invention R¹ is a methyl group substituted with one R²¹ group, and said R²¹ group is phenyl, and said phenyl is substituted with one R²² F group.

In another embodiment of this invention R¹ is selected from the group consisting of:

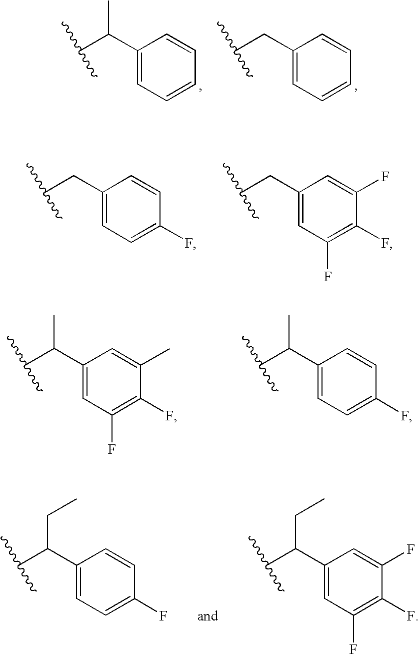

In another embodiment of this invention R¹ is selected from the group consisting of:

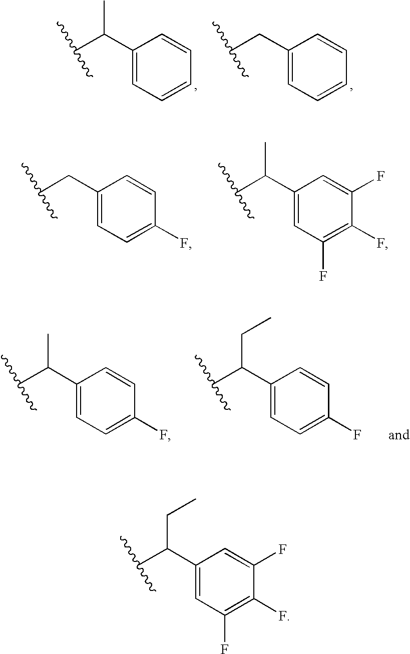

In another embodiment of this invention: (a) R¹ is an alkyl group substituted with one R²¹ group, or (b) R¹ is an alkyl group substituted with one R²¹ group, and said R²¹ group is substituted with one or more independently selected R²² groups, and (c) Ring (B) is any one of the rings described above, and (d) Ring (C) is any one of the rings described above, and (e) R⁹ is selected from the group consisting of heteroaryl and heteroaryl substituted with one or more independently selected R²¹ groups.

In another embodiment of this invention: (a) R¹ is an alkyl group substituted with one phenyl group, or (b) R¹ is an alkyl group substituted with one phenyl group, and said phenyl group is substituted with one or more independently selected R²² groups, and (c) Ring (B) is any one of the rings described above, and (d) Ring (C) is any one of the rings described above, and (e) R⁹ is selected from the group consisting of imidazolyl and imidazolyl substituted with one or more independently selected R²¹ groups.

In another embodiment of this invention: (a) R¹ is a methyl or ethyl group substituted with one phenyl, or (b) R¹ is an methyl or alkyl group substituted with one phenyl, and said phenyl is substituted with one or more independently selected halos, and (c) Ring (B) is any one of the rings described above, and (d) Ring (C) is any one of the rings described above, and (e) R⁹ is selected from the group consisting of imidazolyl and imidazolyl substituted with one or more independently selected alkyl groups groups.

In another embodiment of this invention: (a) R¹ is a methyl or ethyl group substituted with one phenyl, or (b) R¹ is an methyl or alkyl group substituted with one phenyl, and said phenyl is substituted with one or two independently selected halos, and (c) Ring (B) is any one of the rings described above, and (d) Ring (C) is any one of the rings described above, and (e) R⁹ is selected from the group consisting of imidazolyl and imidazolyl substituted with one or two independently selected alkyl groups groups.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) Ring (B) is any one of the rings described above, and (d) Ring (C) is any one of the rings described above, and (e) R⁹ is selected from the group consisting of imidazolyl and imidazolyl substituted with one or two independently selected methyl groups groups.

In another embodiment of this invention: (a) R¹ is a methyl or ethyl group substituted with one phenyl, or (b) R¹ is an methyl or alkyl group substituted with one phenyl, and said phenyl is substituted with one or two F, and (c) Ring (B) is any one of the rings described above, and (d) Ring (C) is any one of the rings described above, and (e) R⁹ is selected from the group consisting of imidazolyl and imidazolyl substituted with one methyl group.

In another embodiment of this invention: (a) R¹ is an alkyl group substituted with one R²¹ group, or (b) R¹ is an alkyl group substituted with one R²¹ group, and said R²¹ group is substituted with one or more independently selected R²² groups, and (c) Ring (B) is any one of the rings described above, and (d) the R⁹—R(C)— moiety is (wherein “R(C)” represents Ring (C)):

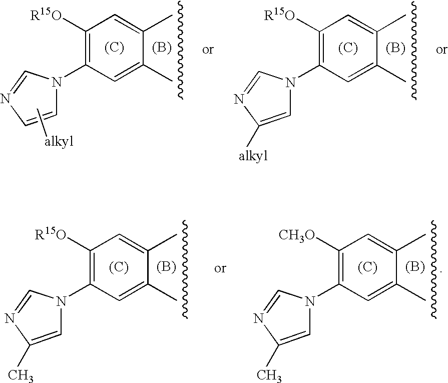

In another embodiment of this invention: (a) R¹ is an alkyl group substituted with one phenyl group, or (b) R¹ is an alkyl group substituted with one phenyl group, and said phenyl group is substituted with one or more independently selected R²² groups, and (c) Ring (B) is any one of the rings described above, (d) the R⁹—R(C)— moiety is:

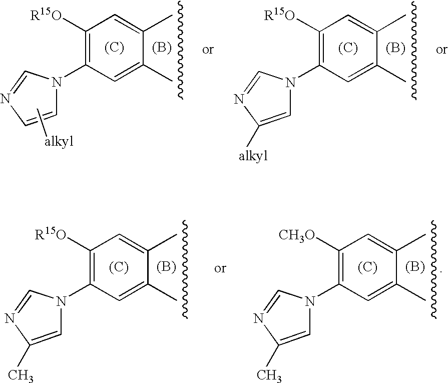

In another embodiment of this invention: (a) R¹ is a methyl or ethyl group substituted with one phenyl, or (b) R¹ is an methyl or alkyl group substituted with one phenyl, and said phenyl is substituted with one or more independently selected halos, and (c) Ring (B) is any one of the rings described above, and (d) the R⁹—R(C)— moiety is:

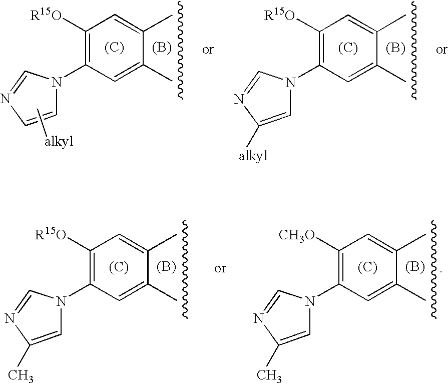

In another embodiment of this invention: (a) R¹ is a methyl or ethyl group substituted with one phenyl, or (b) R¹ is an methyl or alkyl group substituted with one phenyl, and said phenyl is substituted with one or two F, and (c) Ring (B) is any one of the rings described above, and (d) the R⁹—R(C)— moiety is:

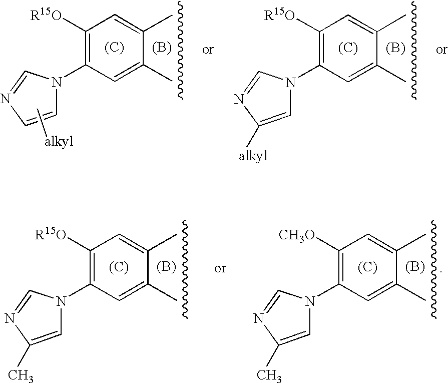

In another embodiment of this invention R¹ is selected from the group consisting of:

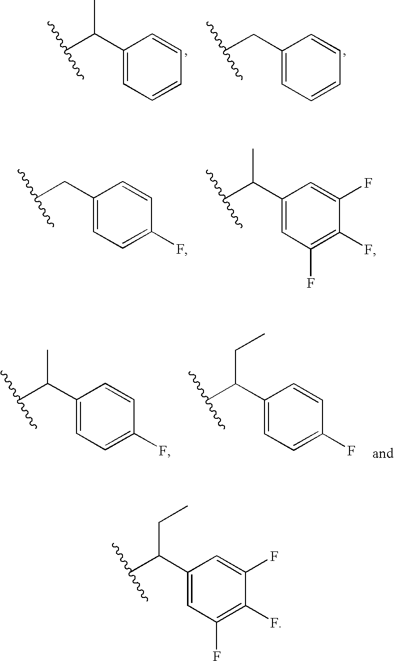

wherein the R⁹-Ring (C)— moiety is:

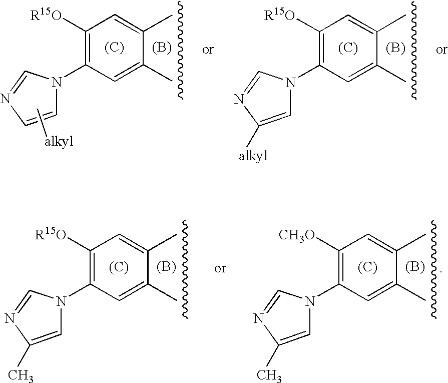

In another embodiment of this invention R¹ is selected from the group consisting of:

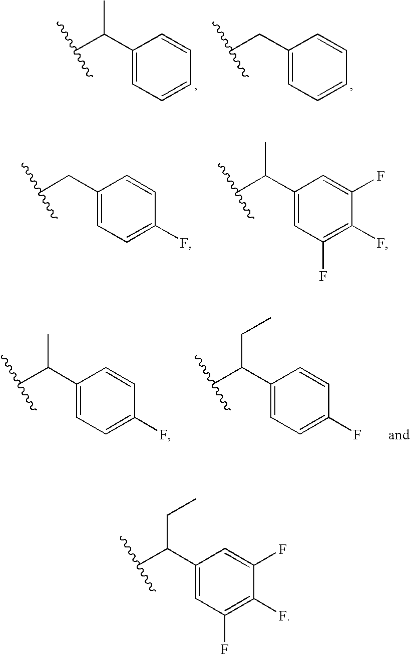

wherein the R⁹-Ring (C)— moiety is:

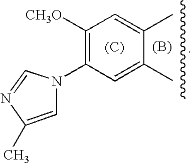

In another embodiment of this invention W is —C(O)—.

In another embodiment of this invention W is —S(O)—.

In another embodiment of this invention W is —S(O)₂—.

In another embodiment of this invention W is —C(═O═NR²)—,

In another embodiment W is —C(═NR²)— wherein R² is selected from the group consisting of: —OH, —O-alkyl (i.e., alkoxy), —O-(halo substituted alky) (such as, for example, —O-fluoroalkyl), —NH(R⁴), —N(R⁴)₂ (wherein each R⁴ is independently selected), —NH₂, —S(R⁴), —S(O)R⁴, —S(O)(OR⁴), —S(O)₂R⁴, —S(O)₂(OR⁴), —S(O)NHR⁴, —S(O)N(R⁴)₂ (wherein each R⁴ is independently selected), —S(O)NH₂, —S(O)₂NHR⁴, —S(O)₂N(R⁴)₂ (wherein each R⁴ is independently selected), —S(O)₂NH₂, —CN, —C(O)₂R⁴, —C(O)NHR⁴, —C(O)N(R⁴)₂ (wherein each R⁴ is independently selected), —C(O)NH₂, and —C(O)R⁴.

Other embodiments of this invention are directed to compounds of formula (I) wherein R¹ is selected from the group consisting of: benzofusedcycloalkyl (i.e., fused benzocycloalkyl), fused benzoheterocycloalkyl, fused heteroarylcycloalkyl, fused heteroarylheterocycloalkyl, and wherein said R¹ groups are optionally substituted with 1-5 independently selected R²¹ groups. In one example, the R²¹ groups are halo (e.g., F).

Examples of the fused ring R¹ groups include, but are not limited to:

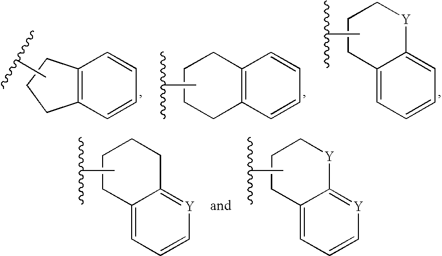

wherein each Y is independently selected from the group consisting of: —O—, —NR¹⁴— and —C(R²¹)_{q}—, wherein q is as defined above (i.e., 0, 1 or 2 and each R²¹ is independently selected), and wherein R¹⁴ and R²¹ are as defined for formula (IL Examples of these R¹ groups include, for example:

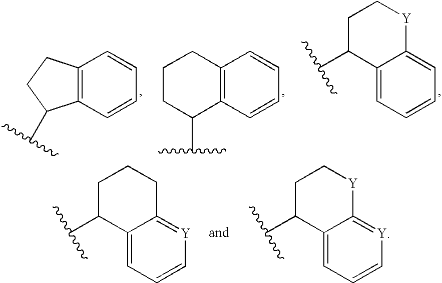

Compounds of formula (I) also include compounds wherein R¹ is an alkyl group (e.g., ethyl) substituted with one R²¹ group, Examples of said R¹ groups include alkyl (e.g., methyl or ethyl) substituted with the R²¹ moiety aryl (e.g., phenyl or naphthyl). Examples of said R¹ groups also include alkyl (e.g., methyl or ethyl) substituted with the R²¹ moiety aryl (e.g., phenyl or naphthyl), which in turn is substituted with one or more (e.g., one or two) independently selected R²² groups (e.g., R²² is halo, such as, for example, F).

Examples of the substituted R¹ alkyl groups include, but are not limited to:

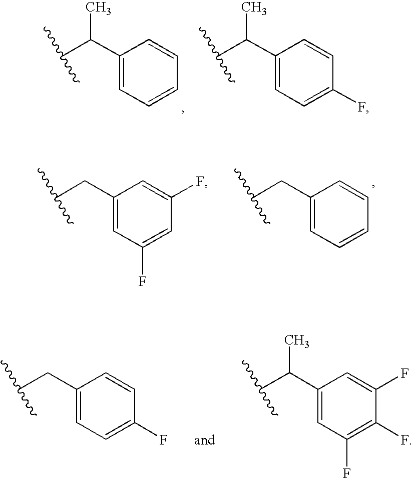

In another embodiment of this invention R¹ is

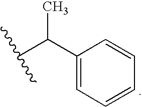

In another embodiment of this invention R¹ is

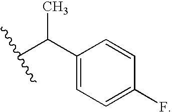

In another embodiment of this invention R¹ is

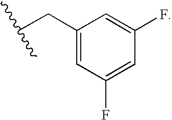

In another embodiment of this invention R¹ is

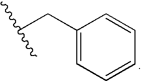

In another embodiment of this invention R¹ is

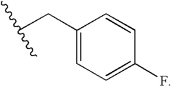

In another embodiment R¹ is

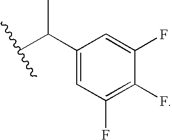

In another embodiment of this invention R¹ is

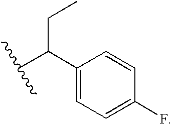

In another embodiment of this invention R¹ is

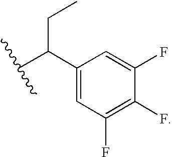

Other embodiments of this invention are directed to compounds of formula (I) wherein R¹ is a cycloalkyl group (e.g., cyclopropyl or cyclobutyl) substituted with one R²¹ group (e.g., aryl, such as, for example, phenyl), or a cycloalkyl group (e.g., cyclopentyl or cyclohexyl) substituted with one R²¹ group (e.g., aryl, such as, for example, phenyl) which in turn is substituted with one or more (e.g., one or two) independently selected R²² groups (e.g., halo, such as, for example, F). In one example the R²¹ group is bound to the same carbon of the R¹ group that binds the R¹ group to the rest of the molecule.

Examples of the cycloalkyl R¹ groups include, but are not limited to:

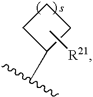

such as, for example,

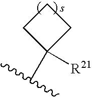

wherein s is 0 (i.e., the ring is cyclopropyl), or 1 (i.e., the ring is cyclobutyl). Examples of these R¹ groups include but are not limited to:

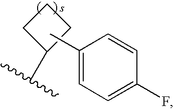

such as, for example,

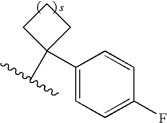

wherein s is 0 (i.e., the ring is cyclopropyl), or 1 (i.e., the ring is cyclobutyl).

Other embodiments of this invention are directed to compounds of formula (I) wherein R¹ is

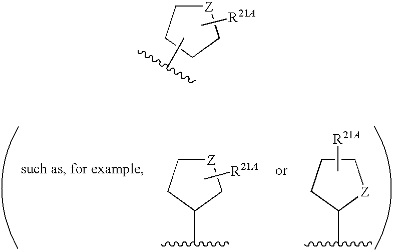

wherein Z is selected from the group consisting of: (1) —O—, (2) —NR¹⁴—, (3) —C(R²¹)_{q}— wherein q is 0, 1 or 2, and each R²¹ is independently selected, (4) —C(R²¹)_{q}—C(R²¹)_{q}— wherein each q is independently 0, 1 or 2 and each R²¹ is indepenendently selected, (5) —(C(R²¹)_{q})_{q}—O—(C(R²¹)_{q})_{q}— wherein each q is independently 0, 1 or 2, and each R²¹ is independently selected, and (6) —(C(R²¹)_{q})_{q}—N(R¹⁴)—(C(R²¹)_{q})_{q}— wherein each q is independently 0, 1 or 2, and each R²¹ is independently selected. R^{21A} is defined the same as R²¹ for formula (I). Examples of R^{21A} include, but are not limited to, aryl (e.g., phenyl) and aryl (e.g., phenyl) substituted with one or more (e.g., one or two, or one) independently selected R²² groups (e.g., halo, such as, for example, F). Examples of this R¹ include, but are not limited to:

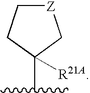

Thus, examples of this R¹ group include, but are not limited to:

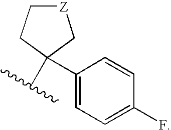

Examples of R¹ also include, but are not limited to:

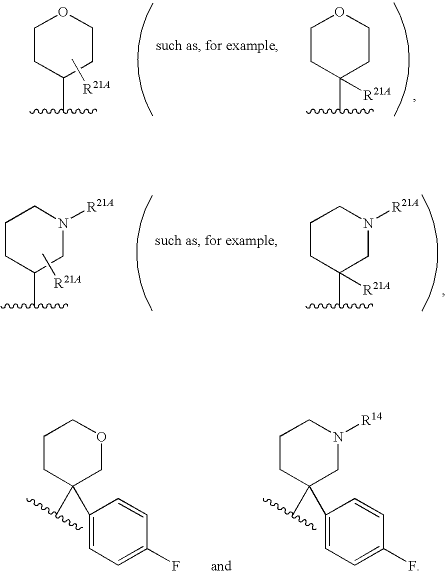

Examples of the R¹ group

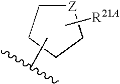

also include, but are not limited to:

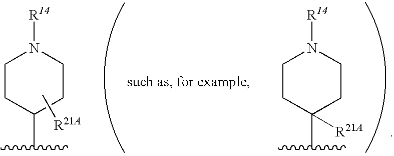

Examples of the R¹ group

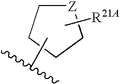

also include, but are not limited to:

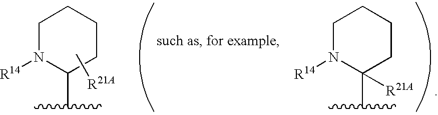

Examples of the R¹ group

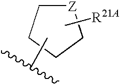

also include, but are not limited to:

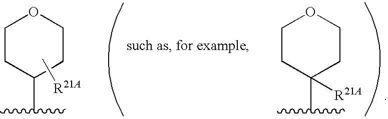

Examples of the R¹ group

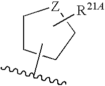

also include, but are not limited to:

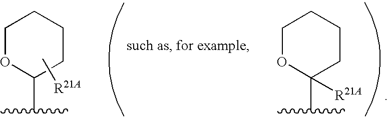

Other embodiments of this invention are directed to the compounds of formula (I) wherein Ring (C) is heteroaryl or heteroaryl substituted with one or more R²¹ groups, and R⁹ is heteroaryl (e.g., imidazolyl) or heteroaryl (e.g., imidazolyl) substituted with one or more (e.g., one or two, or one) R²¹ groups (e.g., alkyl, such as, for example, methyl).

In another embodiment of the compounds of formula (I) R⁹ is heteroaryl.

In another embodiment of the compounds of formula (I) R⁹ is heteroaryl substituted with one or more (e.g., one) independently selected R²¹ groups.

In another embodiment of the compounds of formula (I) R⁹ is heteroaryl substituted with one or more (e.g., one) independently selected R²¹ groups, wherein each R²¹ group is the same or different alkyl group (e.g., methyl).

In another embodiment of the compounds of formula (I) R⁹ is heteroaryl substituted with one R²¹ group.

In another embodiment of the compounds of formula (I) R⁹ is heteroaryl substituted with one R²¹ group, wherein R²¹ is an alkyl group (e.g., methyl).

In another embodiment of the compounds of formula (I) R⁹ is imidazolyl.

In another embodiment of the compounds of formula (I) R⁹ is imidazolyl substituted with one or more (e.g., one) independently selected R²¹ groups.

In another embodiment of the compounds of formula (I) R⁹ is imidazolyl substituted with one or more (e.g., one) independently selected R²¹ groups, wherein each R²¹ group is the same or different alkyl group (e.g., methyl).

In another embodiment of the compounds of formula (I) R⁹ is imidazolyl substituted with one R²¹ group.

In another embodiment of the compounds of formula (I) R⁹ is imidazolyl substituted with one R²¹ group, wherein R²¹ is an alkyl group (e.g., methyl).

In another embodiment of the compounds of formula (I) R⁹ is heteroaryl, optionally substituted with one or more R²¹ groups, Ring (B) is any one of the rings described above, Ring (C) is any one of the rings described above (e.g., phenyl) substituted with one or more (e.g., one) R²¹ groups, and W is —C(O)—. In one example the R²¹ groups for R⁹ are independently selected from alkyl. In another example of this embodiment the R²¹ groups for Ring (C) are independently selected from —OR¹⁵ (wherein, for example, R¹⁵ is alkyl, such as, for example, methyl). In one example of this embodiment R⁹ is substituted with one R²¹ group. In another example of this embodiment Ring (C) is phenyl substituted with one R²¹ group. In another example of this embodiment R⁹ is substituted with one R²¹ group, and Ring (C) is phenyl substituted with one R²¹ group, each R²¹ being independently selected. In another example of this embodiment the R⁹ is substituted with one R²¹ group and said R²¹ group is alkyl (e.g., methyl), and Ring (C) is phenyl substituted with one R²¹ group and this R²¹ group is —OR¹⁵ (wherein R¹⁵ is, for example, alkyl, such as, for example, methyl).

Other embodiments of the compounds of formula (I) are directed to any one of the above embodiments wherein R⁹ is:

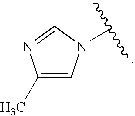

In another embodiment of the compounds of formula (I) R¹ is benzofusedcycloalkyl.

In another embodiment of the compounds of formula (I) R¹ is:

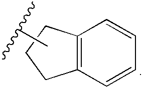

In another embodiment of the compounds of formula (I) R¹ is:

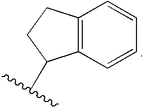

In another embodiment of the compounds of formula (I) R¹ is:

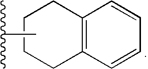

In another embodiment of the compounds of formula (I) is:

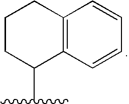

In another embodiment of the compounds of formula (I) R¹ is alkyl substituted with one R²¹ group.

In another embodiment of the compounds of formula (I) R¹ is alkyl substituted with one R²¹ group, and said alkyl is

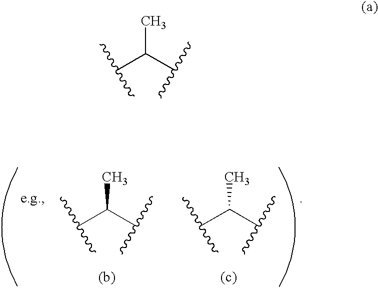

In another embodiment of the compounds of formula (I) R¹ is alkyl (e.g., (a), (b) or (c) described above) substituted with one R²¹ group wherein said R²¹ group is aryl.

In another embodiment of the compounds of formula (I) R¹ is alkyl (e.g., (a), (b) or (c) described above) substituted with one R²¹ group wherein said R²¹ group is phenyl.

In another embodiment of the compounds of formula (I) R¹ is alkyl (e.g., (a), (b) or (c) described above) substituted with one R²¹ group wherein said R²¹ group is naphthyl.

In another embodiment of the compounds of formula (I) R¹ is alkyl substituted with one R²¹ group, and said R²¹ group is substituted with two independently selected R²² groups.

In another embodiment of the compounds of formula (I) R¹ is alkyl substituted with one R²¹ group, and said R²¹ group is substituted with one R²² group.

In another embodiment of the compounds of formula (I) R¹ is alkyl substituted with one R²¹ group, wherein said alkyl group is (a) (e.g., (b) or (c)), as described above, and said R²¹ group is substituted with two independently selected R²² groups.

In another embodiment of the compounds of formula (I) R¹ is alkyl substituted with one R²¹ group, wherein said alkyl group is (a) (e.g., (b) or (c)), as described above, and said R²¹ group is substituted with one R²² group.

In another embodiment of the compounds of formula (I) R¹ is alkyl substituted with one R²¹ group, wherein said R²¹ group is aryl, and said R²¹ group is substituted with two independently selected R²² groups.

In another embodiment of the compounds of formula (I) R¹ is alkyl substituted with one R²¹ group, wherein said R²¹ group is aryl, and said R²¹ group is substituted with one R²² group.

In another embodiment of the compounds of formula (I) R¹ is alkyl substituted with one R²¹ group, wherein said R²¹ group is aryl, said alkyl group is (a) (e.g., (b) or (c)), as described above, and said R²¹ group is substituted with two independently selected R²² groups.

In another embodiment of the compounds of formula (I) R¹ is alkyl substituted with one R²¹ group, wherein said R²¹ group is aryl, wherein said alkyl group is (a) (e.g., (b) or (c)), as described above, and said R²¹ group is substituted with one R²² group.

In another embodiment of the compounds of formula (I) R¹ is alkyl substituted with one R²¹ group, wherein said R²¹ group is aryl, said R²¹ group is substituted with two independently selected R²² groups, and each R²² is halo.

In another embodiment of the compounds of formula (I) R¹ is alkyl substituted with one R²¹ group, wherein said R²¹ group is aryl, and said R²¹ group is substituted with one R²² group, and said R²² is halo.

In another embodiment of the compounds of formula (I) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is halo.

In another embodiment of the compounds of formula (I) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is halo.

In another embodiment of the compounds of formula (I) R¹ is alkyl substituted with one R²¹ group, wherein said R²¹ group is aryl, said R²¹ group is substituted with two independently selected R²² groups, and each R²² is F.

In another embodiment of the compounds of formula (I) R¹ is alkyl substituted with one R²¹ group, wherein said R²¹ group is aryl, and said R²¹ group is substituted with one R²² group, and said R²² is F.

In another embodiment of the compounds of formula (I) R¹ is alkyl substituted with one R²¹ group, wherein said R²¹ group is aryl, said alkyl group is (a) (e.g., (b) or (c)), as described above, and said R²¹ group is substituted with two independently selected R²² groups, and each R²² is F.

In another embodiment of the compounds of formula (I) R¹ is alkyl substituted with one R²¹ group, wherein said R²¹ group is aryl, wherein said alkyl group is (a) (e.g., (b) or (c)), as described above, and said R²¹ group is substituted with one R²² group. and said R²² is F.

In another embodiment of this invention the compound of formula (I) is a compound of the formula:

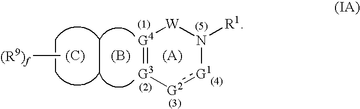

In another embodiment of this invention the compound of formula (I) is a compound of the formula:

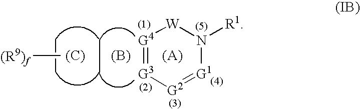

In another embodiment of this invention the compound of formula (I) is a compound of the formula:

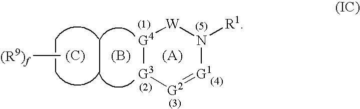

In another embodiment of this invention the compound of formula (I) is a compound of the formula:

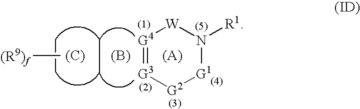

In another embodiment of this invention the compound of formula (I) is a compound of the formula:

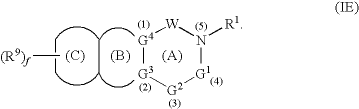

In another embodiment of this invention the compound of formula (I) is a compound of the formula:

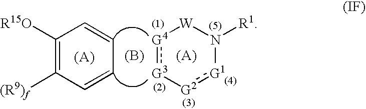

In another embodiment of this invention the compound of formula (I) is a compound of the formula:

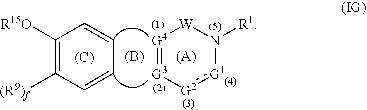

In another embodiment of this invention the compound of formula (I) is a compound of the formula:

In another embodiment of this invention the compound of formula (I) is a compound of the formula:

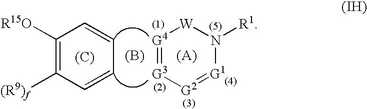

In another embodiment of this invention the compound of formula (I) is a compound of the formula:

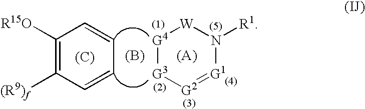

In another embodiment of this invention the compound of formula (I) is a compound of the formula:

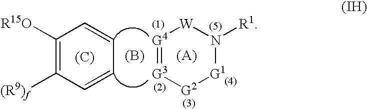

In another embodiment of this invention the compound of formula (I) is a compound of the formula:

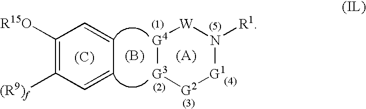

In another embodiment of this invention the compound of formula (I) is a compound of the formula:

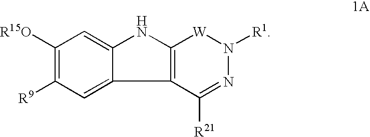

In another embodiment of this invention the compound of formula (I) is a compound of the formula:

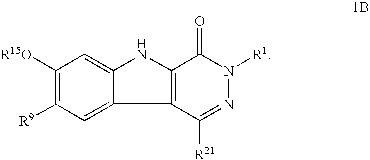

In another embodiment of this invention the compound of formula (I) is a compound of the formula:

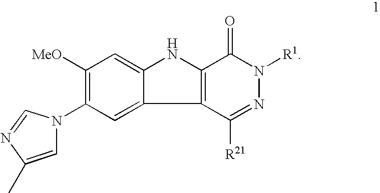

In another embodiment of this invention the compound of formula (I) is a compound of the formula:

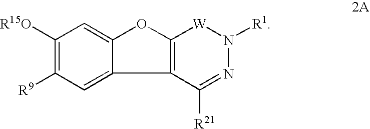

In another embodiment of this invention the compound of formula (I) is a compound of the formula:

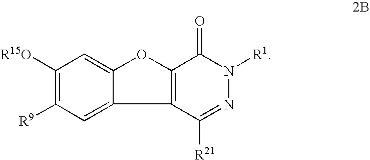

In another embodiment of this invention the compound of formula (I) is a compound of the formula:

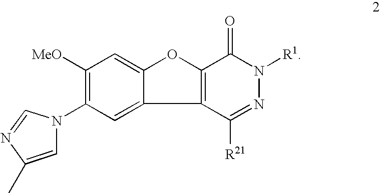

In another embodiment of this invention the compound of formula (I) is a compound of the formula:

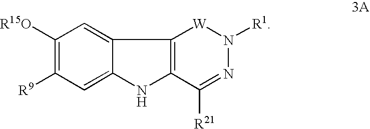

In another embodiment of this invention the compound of formula (I) is a compound of the formula:

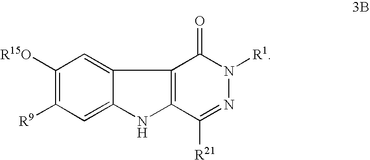

In another embodiment of this invention the compound of formula (I) is a compound of the formula:

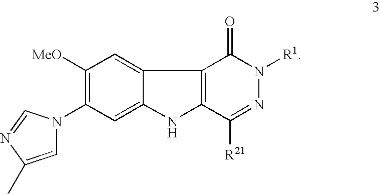

In another embodiment of this invention the compound of formula (I) is a compound of the formula:

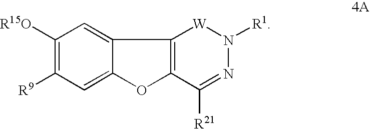

In another embodiment of this invention the compound of formula (I) is a compound of the formula:

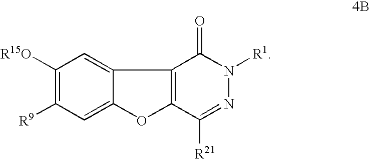

In another embodiment of this invention the compound of formula (I) is a compound of the formula:

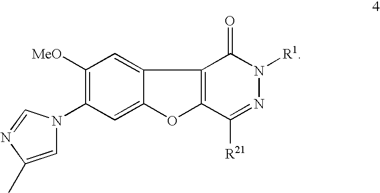

In another embodiment of this invention the compound of formula a compound of the formula:

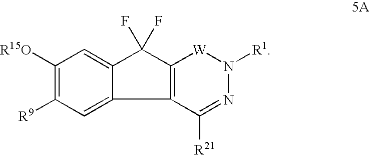

In another embodiment of this invention the compound of formula (I) is a compound of the formula:

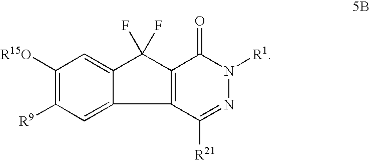

In another embodiment of this invention the compound of formula (I) is a compound of the formula;

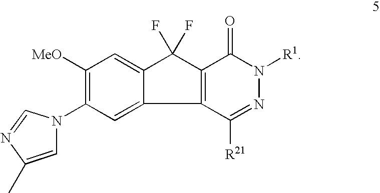

In another embodiment of this invention the compound of formula (I) is a compound of the formula:

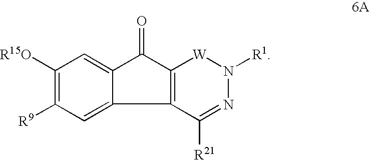

In another embodiment of this invention the compound of formula (I) is a compound of the formula:

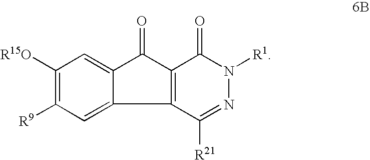

In another embodiment of this invention the compound of formula (I) is a compound of the formula:

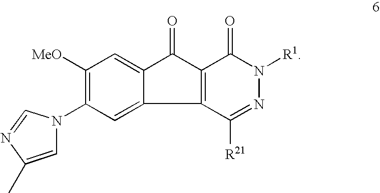

In another embodiment of this invention the compound of formula (I) is a compound of the formula:

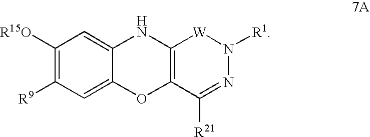

In another embodiment of this invention the compound of formula (I) is a compound of the formula:

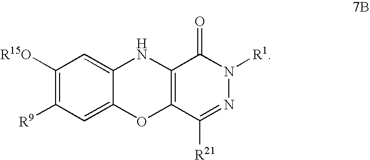

In another embodiment of this invention the compound of formula (I) is a compound of the formula:

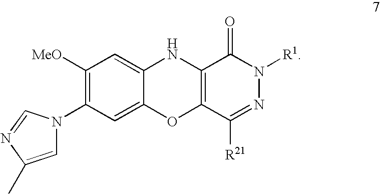

In another embodiment of this invention the compound of formula (I) is a compound of the formula:

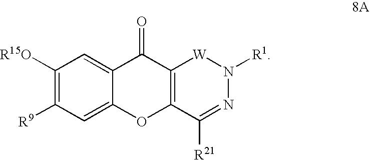

In another embodiment of this invention the compound of formula (I) is a compound of the formula:

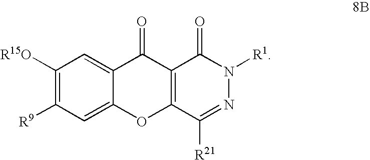

In another embodiment of this invention the compound of formula (0 is a compound of the formula:

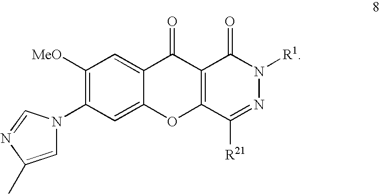

In another embodiment of this invention the compound of formula (I) is a compound of the formula:

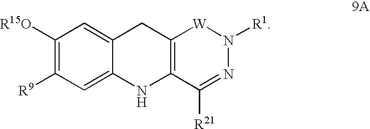

In another embodiment of this invention the compound of formula (I) is a compound of the formula:

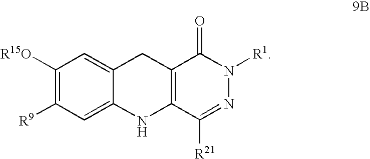

In another embodiment of this invention the compound of formula (I) is a compound of the formula:

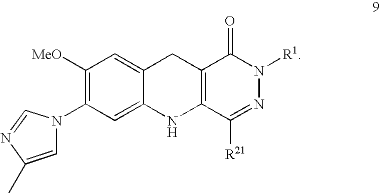

In another embodiment of this invention the compound of formula a compound of the formula:

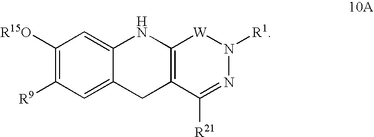

In another embodiment of this invention the compound of formula (I) is a compound of the formula:

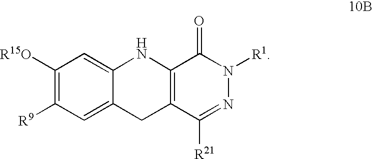

In another embodiment of this invention the compound of formula (I) is a compound of the formula:

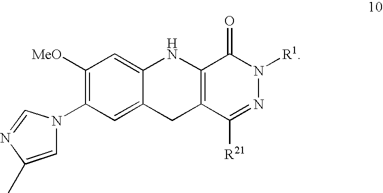

In another embodiment of this invention the compound of formula (I) is a compound of the formula:

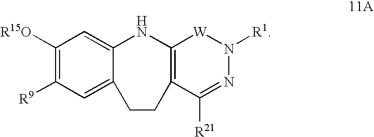

In another embodiment of this invention the compound of formula (I) is a compound of the formula:

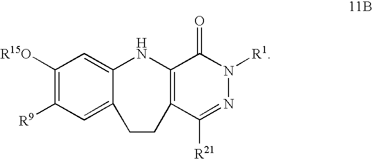

In another embodiment of this invention the compound of formula (I) is a compound of the formula:

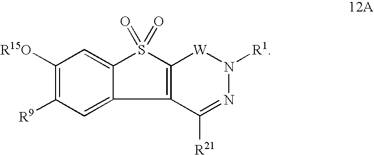

In another embodiment of this invention the compound of formula a compound of the formula:

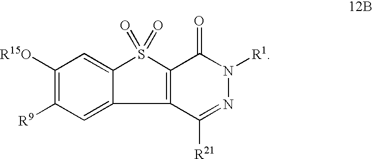

In another embodiment of this invention the compound of formula (I) is a compound of the formula:

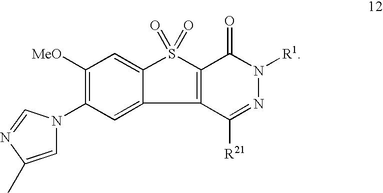

In another embodiment of this invention the compound of formula (I) is a compound of the formula:

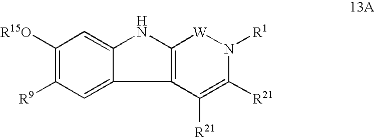

wherein each R²¹ is independently selected.

In another embodiment of this invention the compound of formula (I) is a compound of the formula:

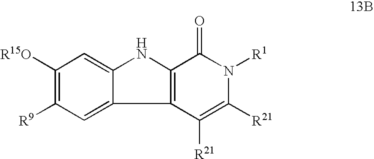

wherein each R²¹ is independently selected.

In another embodiment of this invention the compound of formula is a compound of the formula:

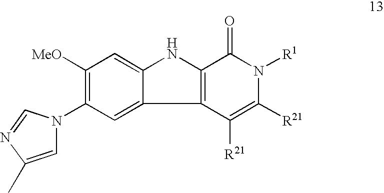

wherein each R²¹ is independently selected.

In another embodiment of this invention the compound of formula (I) is a compound of the formula:

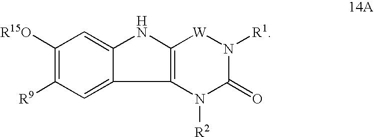

In another embodiment of this invention the compound of formula (I) is a compound of the formula:

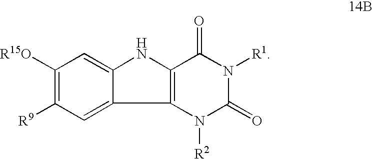

In another embodiment of this invention the compound of formula (I) is a compound of the formula:

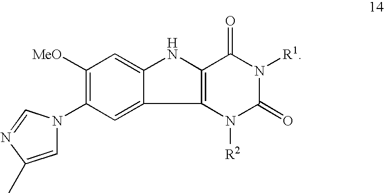

In another embodiment of this invention the compound of formula (I) is a compound of the formula:

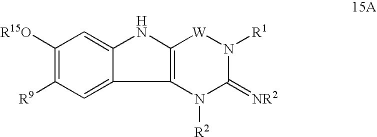

wherein each R² is independently selected.

In another embodiment of this invention the compound of formula (I) a compound of the formula:

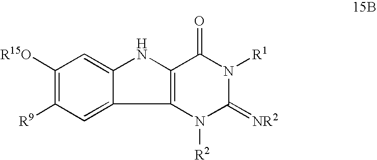

wherein each R independently selected.

In another embodiment of this invention the compound of formula (I) is a compound of the formula:

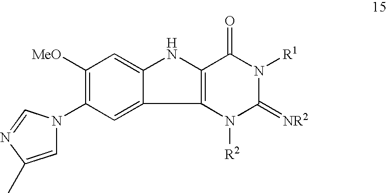

wherein each R² is independently selected.

In another embodiment of this invention the compound of formula (I) is a compound of the formula:

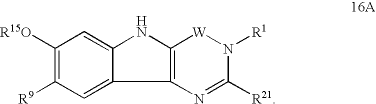

In another embodiment of this invention the compound of formula (I) is a compound of the formula:

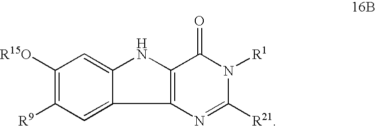

In another embodiment of this invention the compound of formula (I) is a compound of the formula:

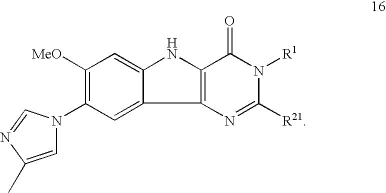

In another embodiment of this invention the compound of formula (I) is a compound of the formula:

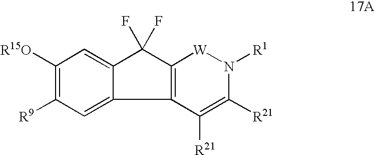

wherein each R²¹ is independently selected.

In another embodiment of this invention the compound of formula (I) is a compound of the formula:

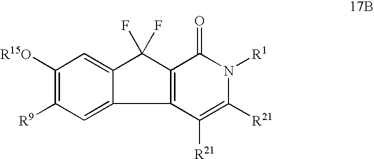

wherein each R²¹ is independently selected.

In another embodiment of this invention the compound of formula (I) is a compound of the formula:

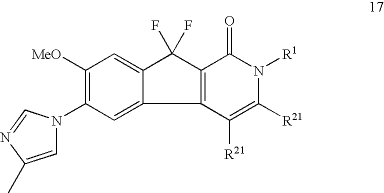

wherein each R²¹ is independently selected.

In another embodiment of this invention the compound of formula (I) is a compound of the formula:

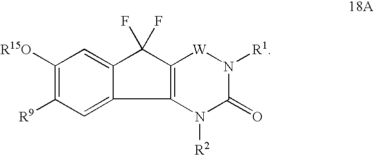

In another embodiment of this invention the compound of formula (I) is a compound of the formula:

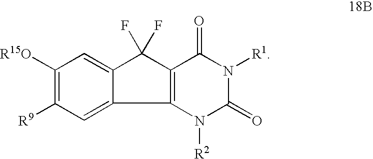

In another embodiment of this invention the compound of formula (I) is a compound of the formula:

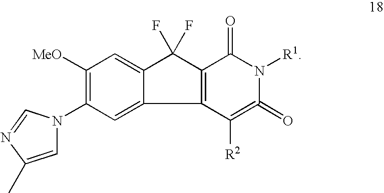

In another embodiment of this invention the compound of formula (I) is a compound of the formula:

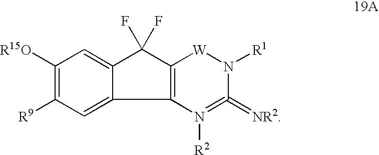

wherein each R² is independently selected.

In another embodiment of this invention the compound of formula (I) is a compound of the formula:

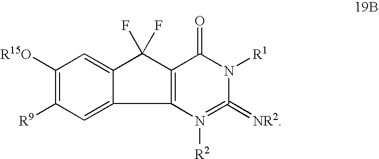

wherein each R² is independently selected.

In another embodiment of this invention the compound of formula (I) is a compound of the formula:

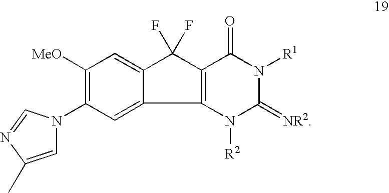

wherein each R² is independently selected.

In another embodiment of this invention the compound of formula (I) is a compound of the formula:

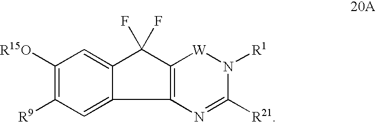

In another embodiment of this invention the compound of formula (I) is a compound of the formula:

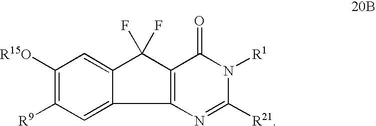

In another embodiment of this invention the compound of formula (I) is a compound of the formula:

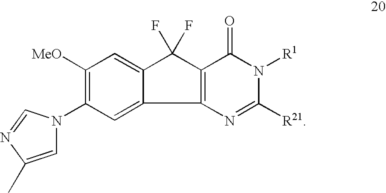

In another embodiment of this invention the compound of formula (I) is a compound of the formula:

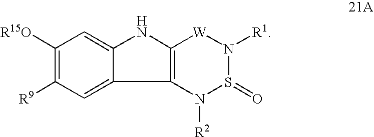

In another embodiment of this invention the compound of formula (I) is a compound of the formula:

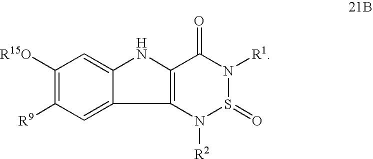

In another embodiment of this invention the compound of formula (I) is a compound of the formula:

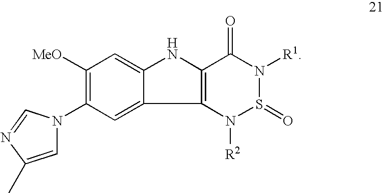

In another embodiment of this invention the compound of formula (I) is a compound of the formula:

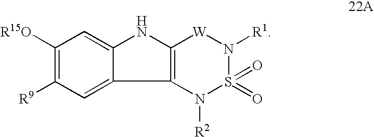

In another embodiment of this invention the compound of formula (I) is a compound of the formula:

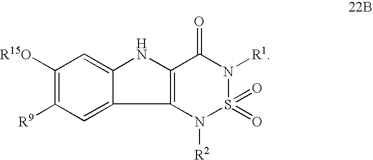

In another embodiment of this invention the compound of formula (I) is a compound of the formula:

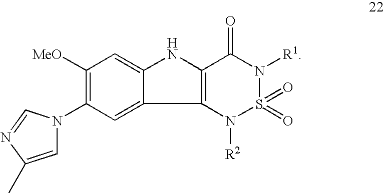

In another embodiment of this invention the compound of formula (I) is a compound of the formula:

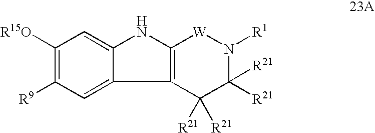

wherein each R²¹ is independently selected.

In another embodiment of this invention the compound of formula (I) is a compound of the formula:

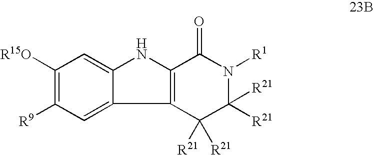

wherein each R²¹ is independently selected.

In another embodiment of this invention the compound of formula (I) is a compound of the formula:

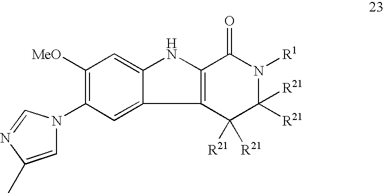

wherein each R²¹ is independently selected,

In another embodiment of this invention the compound of formula (I) is a compound of the formula:

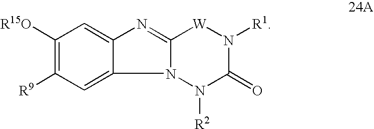

In another embodiment of this invention the compound of formula (I) is a compound of the formula:

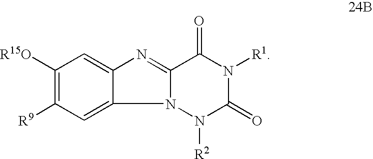

In another embodiment of this invention the compound of formula (I) is a compound of the formula:

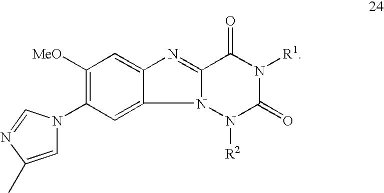

In another embodiment of this invention the compound of formula (I) is a compound of the formula:

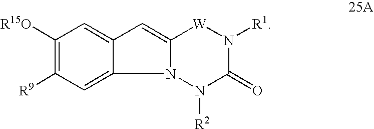

In another embodiment of this invention the compound of formula (I) is a compound of the formula:

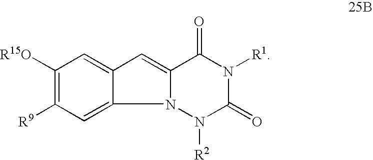

In another embodiment of this invention the compound of formula a compound of the formula:

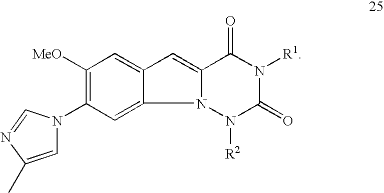

In another embodiment of this invention the compound of formula (I) is a compound of the formula:

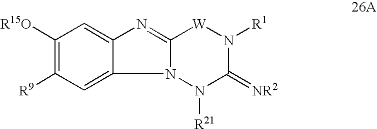

wherein each R² is independently selected.

In another embodiment of this invention the compound of formula (I) is a compound of the formula:

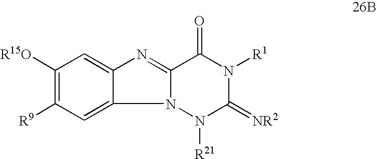

wherein each R² is independently selected.

In another embodiment of this invention the compound of formula (I) is a compound of the formula:

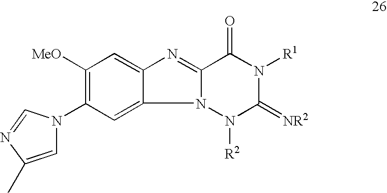

wherein each R² is independently selected.

In another embodiment of this invention the compound of formula (I) is a compound of the formula:

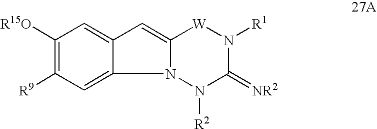

wherein each R² is independently selected.

In another embodiment of this invention the compound of formula (I) is a compound of the formula:

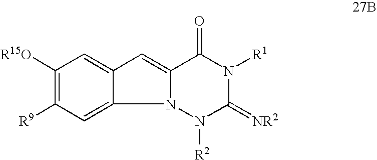

wherein each R² is independently selected.

In another embodiment of this invention the compound of formula (I) is a compound of the formula:

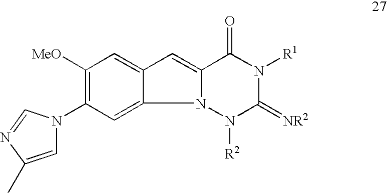

wherein each R² is independently selected.

In another embodiment of this invention the compound of formula (I) is a compound of the formula:

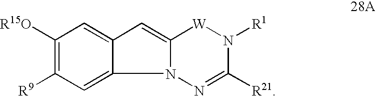

In another embodiment of this invention the compound of formula (I) is a compound of the formula:

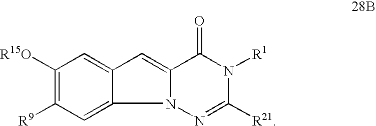

In another embodiment of this invention the compound of formula is a compound of the formula:

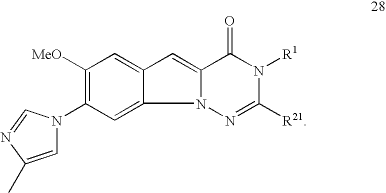

In another embodiment of this invention the compound of formula (I) is a compound of the formula:

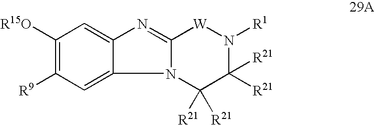

wherein each R²¹ is independently selected.

In another embodiment of this invention the compound of formula (I) is a compound of the formula:

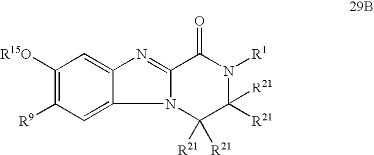

wherein each R²¹ is independently selected.

In another embodiment of this invention the compound of formula (I) is a compound of the formula:

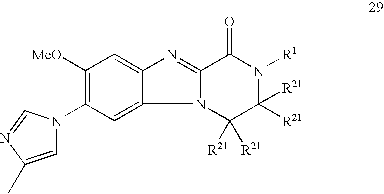

wherein each R²¹ is independently selected.

In another embodiment of this invention the compound of formula (I) is a compound of the formula:

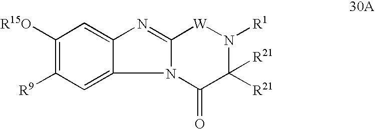

wherein each R²¹ is independently selected.

In another embodiment of this invention the compound of formula (I) is a compound of the formula:

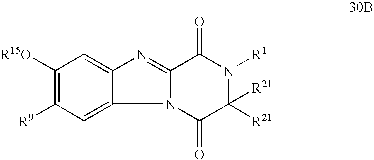

wherein each R²¹ is independently selected.

In another embodiment of this invention the compound of formula (I) is a compound of the formula:

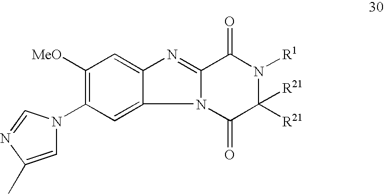

wherein each R²¹ is independently selected.

In another embodiment of this invention the compound of formula (I) is a compound of the formula:

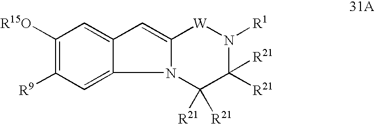

wherein each R²¹ is independently selected,

In another embodiment of this invention the compound of formula (I) is a compound of the formula:

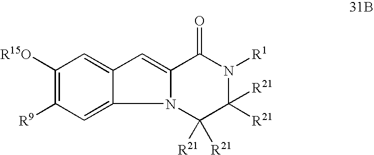

wherein each R²¹ is independently selected.

In another embodiment of this invention the compound of formula (I) is a compound of the formula:

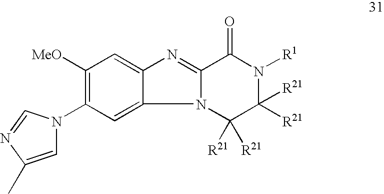

wherein each R²¹ is independently selected.

In another embodiment of this invention the compound of formula (I) is a compound of the formula:

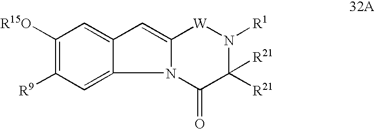

wherein each R²¹ is independently selected.

In another embodiment of this invention the compound of formula (I) is a compound of the formula:

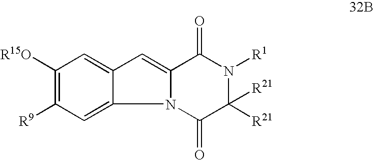

wherein each R²¹ is independently selected.

In another embodiment of this invention the compound of formula (I) is a compound of the formula:

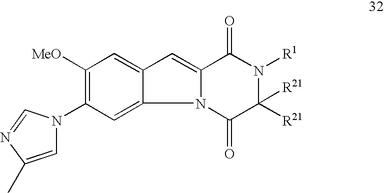

wherein each R²¹ is independently selected.

In another embodiment of this invention the compound of formula (I) is a compound of the formula:

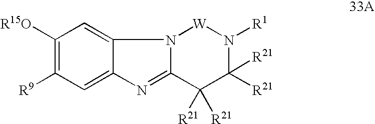

wherein each R²¹ is independently selected.

In another embodiment of this invention the compound of formula (I) is a compound of the formula:

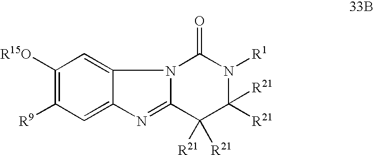

wherein each R²¹ is independently selected.

In another embodiment of this invention the compound of formula (I) is a compound of the formula:

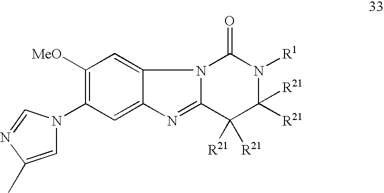

wherein each R²¹ is independently selected.

In another embodiment of this invention the compound of formula (I) is a compound of the formula:

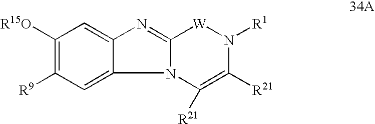

wherein each R²¹ is independently selected.

In another embodiment of this invention the compound of formula (I) is a compound of the formula:

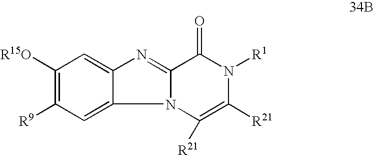

wherein each R²¹ is independently selected.

In another embodiment of this invention the compound of formula (I) is a compound of the formula:

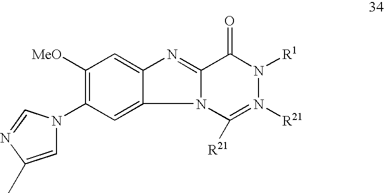

wherein each R²¹ is independently selected.

In another embodiment of this invention the compound of formula (I) is a compound of the formula:

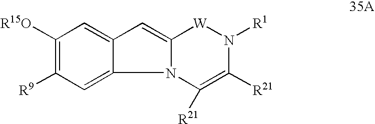

wherein each R²¹ is independently selected.

In another embodiment of this invention the compound of formula (I) is a compound of the formula:

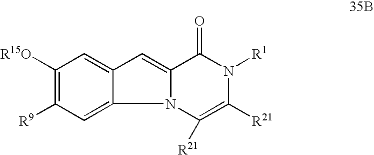

wherein each R²¹ is independently selected.

In another embodiment of this invention the compound of formula (I) is a compound of the formula:

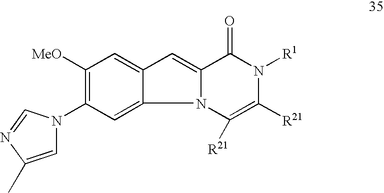

wherein each R²¹ is independently selected.

In another embodiment of this invention the compound of formula (I) is a compound of the formula:

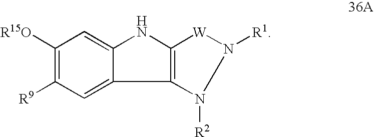

In another embodiment of this invention the compound of formula (I) is a compound of the formula:

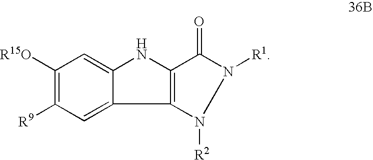

In another embodiment of this invention the compound of formula (I) is a compound of the formula:

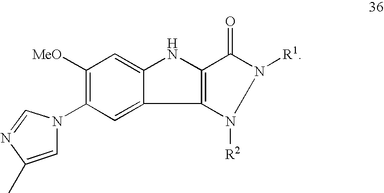

In another embodiment of this invention the compound of formula (I) is a compound of the formula:

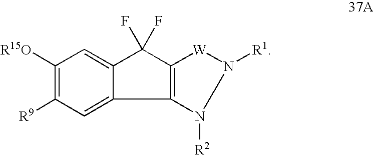

In another embodiment of this invention the compound of formula (I) is a compound of the formula:

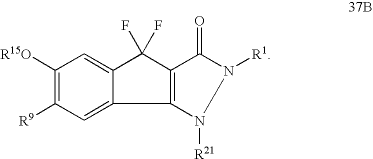

In another embodiment of this invention the compound of formula (I) is a compound of the formula:

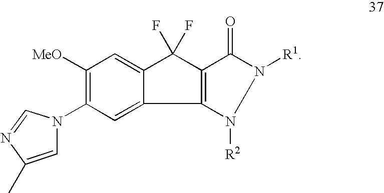

In another embodiment of this invention the compound of formula (I) is a compound of the formula:

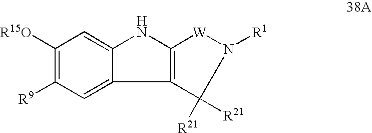

wherein each R²¹ is independently selected.

In another embodiment of this invention the compound of formula (I) is a compound of the formula:

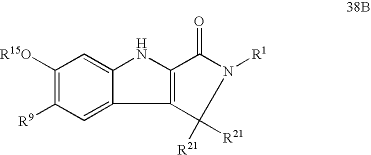

wherein each R²¹ is independently selected.

In another embodiment of this invention the compound of formula (I) is a compound of the formula:

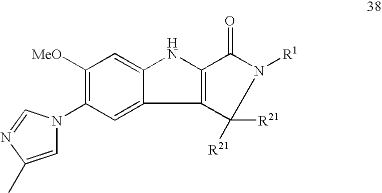

wherein each R²¹ is independently selected.

In another embodiment of this invention the compound of formula (I) is a compound of the formula:

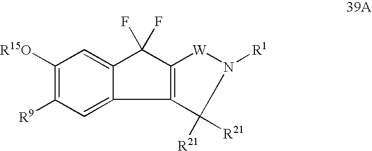

wherein each R²¹ is independently selected.

In another embodiment of this invention the compound of formula (I) is a compound of the formula:

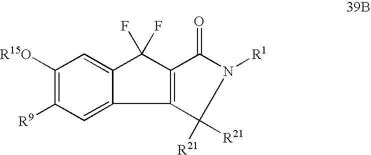

wherein each R²¹ is independently selected.

In another embodiment of this invention the compound of formula (I) is a compound of the formula:

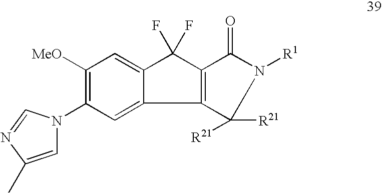

wherein each R²¹ is independently selected.

In another embodiment of this invention the compound of formula (I) is a compound of the formula:

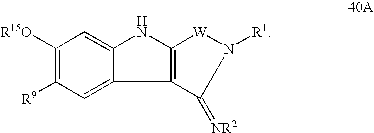

In another embodiment of this invention the compound of formula (I) is a compound of the formula:

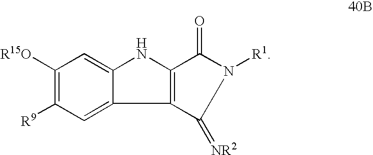

In another embodiment of this invention the compound of formula (I) is a compound of the formula:

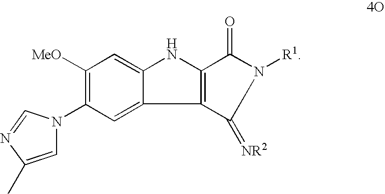

In another embodiment of this invention the compound of formula (I) is a compound of the formula:

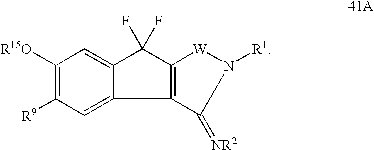

In another embodiment of this invention the compound of formula (I) is a compound of the formula:

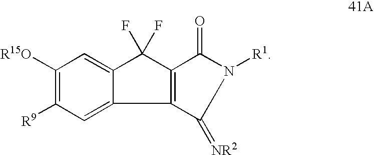

In another embodiment of this invention the compound of formula (I) is a compound of the formula:

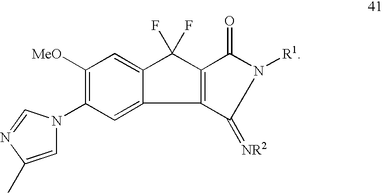

In another embodiment of this invention the compound of formula (I) is a compound of the formula:

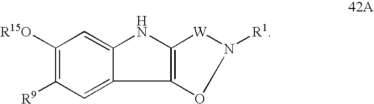

In another embodiment of this invention the compound of formula (I) is a compound of the formula:

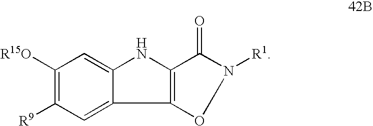

In another embodiment of this invention the compound of formula (I) is a compound of the formula:

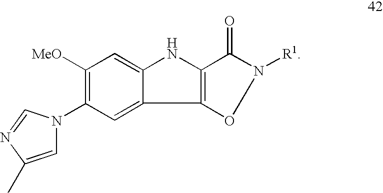

In another embodiment of this invention the compound of formula (I) is a compound of the formula:

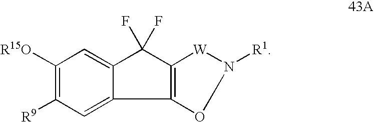

In another embodiment of this invention the compound of formula (I) is a compound of the formula:

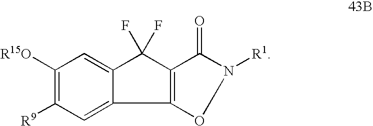

In another embodiment of this invention the compound of formula (I) is a compound of the formula:

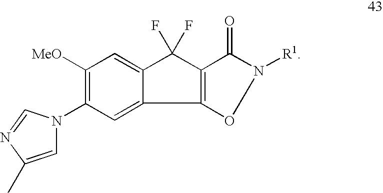

In another embodiment of this invention the compound of formula (I) is a compound of the formula:

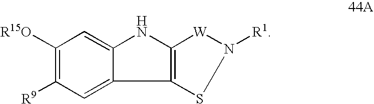

In another embodiment of this invention the compound of formula (I) is a compound of the formula:

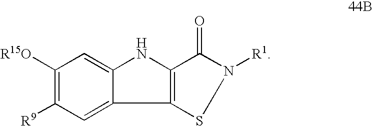

In another embodiment of this invention the compound of formula (I) is a compound of the formula:

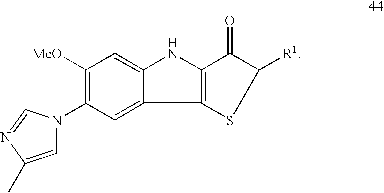

In another embodiment of this invention the compound of formula (I) is a compound of the formula:

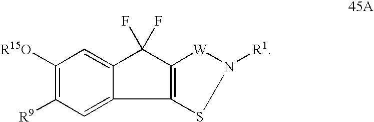

In another embodiment of this invention the compound of formula (I) is a compound of the formula:

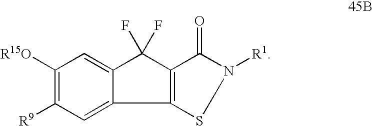

In another embodiment of this invention the compound of formula (I) is a compound of the formula:

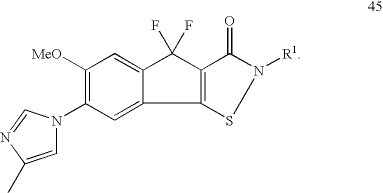

Other embodiments of this invention are directed to any one of the embodiments directed to the compounds of formulas 1 to 45, 1A to 45A, and 1B to 45B wherein R¹ is as defined above in any one of the above embodiments of formula (I).

Other embodiments of this invention are directed to any one of the embodiments directed to the compounds of formulas 1A to 45A, and 1B to 45B wherein R¹, R⁹ and —OR¹⁵ are as defined above in any one of the embodiments of formula (I).

Other embodiments of this invention are directed to any one of the embodiments directed to the compounds of formulas 1A to 45A, and 18 to 45B wherein R¹, R⁹, and —OR¹⁵ are as defined above in any one of the embodiments of formula (I), and R²¹ for compounds 1 to 14, 16, 17, 20, 23, 28-35, 38-39, 1A to 14A, 16A, 17A, 20A, 23A, 28A-35A, 38A-39A, 1B to 14B, 16B, 178, 20B, 23B, 28B-35B, and 388-398 are as defined above in any one of the embodiments of formula (I), and R² for compounds 15, 18, 19, 21, 22, 24, 25, 26, 27, 36, 37, 40, 41, 15A, 18A, 19A, 21A, 22A, 24A, 25A, 26A, 27A, 36A, 37A, 40A, 41A, 15B, 18B, 198, 21B, 22B, 24B, 25B, 26B, 27B, 36B, 37B, 40B, and 41B are as defined above in any one of the embodiments of formula (I).

Examples of R²¹ groups include —OR¹⁵ wherein, for example, R¹⁵ is alkyl (such as methyl or ethyl), or R¹⁵ is cycloalkylalkyl (such as, for example, —CH₂-cyclopropyl), or R¹⁵ is -alkyl-(R¹⁸)ₙ (wherein, for example, said R¹⁸ is —OR²⁰, and said R²⁰ is alkyl, and wherein examples of said -alkyl-(R¹⁸)ₙ moiety is —(CH₂)₂OCH₃).

Examples of R²¹ also include —C(O)OR¹⁵ wherein, for example, A¹⁵ is alkyl, such as, for example, methyl).

Examples of R²¹ also include —C(O)NR¹⁵R¹⁶, wherein, for example, one of R¹⁵ or R¹⁶ is H, and the other is selected from the group consisting of: (R¹⁸)-arylalkyl-, (R¹⁸)ₙ-alkyl-, and cycloalkyl. In one example of this —C(O)NR¹⁵R¹⁶ moiety the R¹⁸ is −OR²⁰, n is 1, R²⁰ is alkyl, said cycloalkyl is cyclobutyl, and said arylalkyl- is benzyl.

Examples of R²¹ also include halo (e.g., Br, Cl or F).

Examples of R²¹ also include arylalkyl, such as, for example, benzyl.

Another embodiment of this invention is directed to a compound of formula (I) selected from the group consisting of: (IA) to (IH), (IJ) to (IL), 1 to 45, 1A to 45A, and 1B to 45B.

Another embodiment of this invention is directed to a pharmaceutically acceptable salt of a compound of formula (I), said compound of formula (I) being selected from the group consisting of: (IA) to (IH), (IJ) to (IL). 1 to 45, 1A to 45A, and 1B to 45B.

Another embodiment of this invention is directed to a pharmaceutically acceptable ester of a compound of formula (I), said compound of formula (I) being selected from the group consisting of: (IA) to (IH), (IJ) to (IL), 1 to 45, 1A to 45A, and 1B to 45B.

Another embodiment of this invention is directed to a solvate of a compound of formula (I), said compound of formula (I) being selected from the group consisting of: (IA) to (IH), (IJ) to (IL), 1 to 45, 1A to 45A, and 16 to 45B.

Another embodiment of this invention is directed to a compound of formula (I) in isolated form, said compound of formula (I) being selected from the group consisting of: (IA) to (IH), (IJ) to (IL), 1 to 45, 1A to 45A, and 1B to 45B.

Another embodiment of this invention is directed to a compound of formula (I) in pure form, said compound of formula (I) being selected from the group consisting of: (IA) to (IH), (IJ) to (IL), 1 to 45, 1A to 45A, and 1B to 45B.

Another embodiment of this invention is directed to a compound of formula (I) in pure and isolated form, said compound of formula (I) being selected from the group consisting of: (IA) to (1H), (IJ) to (IL), 1 to 45, 1A to 45A, and 1B to 45B.

Another embodiment is directed to a pharmaceutical composition comprising an effective amount of one or more (e.g., one) compounds of formula (I) and a pharmaceutically acceptable carrier, said compound of formula (I) being selected from the group consisting of: (IA) to (IH), (IJ) to (IL), 1 to 45, 1 A to 45A, and 1B to 45B.

Another embodiment is directed to a pharmaceutical composition comprising an effective amount of a pharmaceutically acceptable salt of one or more (e.g., one) compounds of formula (I) and a pharmaceutically acceptable carrier, said compound of formula (I) being selected from the group consisting of: (IA) to (1H), (1J) to (IL), 1 to 45, 1A to 45A, and 1B to 45B.

Another embodiment is directed to a pharmaceutical composition comprising an effective amount of a pharmaceutically acceptable ester of one or more (e.g., one) compounds of formula (I) and a pharmaceutically acceptable carrier, said compound of formula (I) being selected from the group consisting of: (IA) to (IH), (IJ) to (IL), 1 to 45, 1A to 45A, and 1B to 45B.

Another embodiment is directed to a pharmaceutical composition comprising an effective amount of a solvate of one or more (e.g., one) compounds of formula (I) and a pharmaceutically acceptable carrier, said compound of formula (I) being selected from the group consisting of: (IA) to (1H), (IJ) to (IL), 1 to 45, 1A to 45A, and 1B to 45B.

Another embodiment of this invention is directed to a pharmaceutical composition comprising an effective amount of one or more (e.g., one) compounds of formula (I), and an effective amount of one or more (e.g., one) other pharmaceutically active ingredients (e.g.,) drugs, and a pharmaceutically acceptable carrier. Examples of the other pharmaceutically active ingredients include, but are not limited to drugs selected form the group consisting of: (a) drugs useful for the treatment of Aizheimer's disease, (b) drugs useful for inhibiting the deposition of amyloid protein (e.g., amyloid beta protein) in, on or around neurological tissue (e.g., the brain), (c) drugs useful for treating neurodegenerative diseases, and (d) drugs useful for inhibiting gamma-secretase, said compound of formula (I) being selected from the group consisting of: (IA) to (IH), (IJ) to (IL), 1 to 45, 1A to 45A, and 1B to 45B.

Another embodiment of this invention is directed to a pharmaceutical composition comprising an effective amount of one or more (e.g., one) compounds of formula (I), and effective amount of one or more BADE inhibitors, and a pharmaceutically acceptable carrier, said compound of formula (I) being selected from the group consisting of: (IA) to (IH), (IJ) to (IL), 1 to 45, 1A to 45A, and 1B to 458.

Another embodiment of this invention is directed to a pharmaceutical composition comprising an effective amount of one or more (e.g., one) compounds selected from the group consisting of: (IA) to (IH), (IJ) to (IL), 1 to 45, 1A to 45A, and 1B to 45B, and effective amount of one or more cholinesterase inhibitors (e.g., acetyl- and/or butyrylchlolinesterase inhibitors), and a pharmaceutically acceptable carrier.

Another embodiment of this invention is directed to a pharmaceutical composition comprising an effective amount of one or more (e.g., one) compounds selected from the group consisting of: (IA) to (IH), (IJ) to (IL), 1 to 45, 1A to 45A, and 1B to 45B, and effective amount of one or more muscarinic antagonists (e.g., m₁ agonists or m₂ antagonists), and a pharmaceutically acceptable carrier.

The compounds of formulas (IA) to (IH), (IJ) to (IL), 1 to 45, 1A to 45A, and 1B to 458 can be useful as gamma secretase modulators and can be useful in the treatment and prevention of diseases such as, for example, central nervous system disorders such as Alzheimers disease and Downs Syndrome.

Thus, another embodiment of this invention is directed to a method for modulating (including inhibiting, antagonizing and the like) gamma-secretase comprising administering an effective (i.e., therapeutically effective) amount of one or more (e.g., one) compounds of formula (I) to a patient in need of such treatment, said compound of formula (I) being selected from the group consisting of: (IA) to (IH), (IJ) to (IL), 1 to 45, 1A to 45A, and 1B to 456.

Another embodiment of this invention is directed to a method for modulating (including inhibiting, antagonizing and the like) gamma-secretase, comprising administering an effective (i.e., therapeutically effective) amount of a compound of formula (I) to a patient in need of treatment, said compound of formula (I) being selected from the group consisting of: (IA) to (IH), (IJ) to (IL), 1 to 45, 1 A to 45A, and 1B to 45B.

Another embodiment of this invention is directed to a method of treating one or more neurodegenerative diseases, comprising administering an effective (i.e., therapeutically effective) amount of one or more (e.g., one) compounds of formula (I) to a patient in need of treatment, said compound of formula (I) being selected from the group consisting of: (IA) to (IH), (IJ) to (IL), 1 to 45, 1A to 45A, and 1B to 45B.

Another embodiment of this invention is directed to a method of treating one or more neurodegenerative diseases, comprising administering an effective (i.e., therapeutically effective) amount of a compound of formula (I) to a patient in need of treatment, said compound of formula (I) being selected from the group consisting of: (IA) to (IH), (IJ) to (IL), 1 to 45, 1A to 45A, and 1B to 45B.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of one or more (e.g., one) compounds of formula (I) to a patient in need of treatment, said compound of formula (I) being selected from the group consisting of: (IA) to (1H), (IJ) to (IL), 1 to 45, 1A to 45A, and 1B to 45B.

Another embodiment of this invention is directed to a method of inhibiting the deposition of amyloid protein (e.g., amyloid beta protein) in, on or around neurological tissue (e.g., the brain), comprising administering an effective (i.e., therapeutically effective) amount of a compound of formula (I) to a patient in need of treatment, said compound of formula (I) being selected from the group consisting of: (IA) to (IH), (IJ) to (IL), 1 to 45, 1A to 45A, and 1B to 45B.

Another embodiment of this invention is directed to a method of treating Alzheimer's disease, comprising administering an effective (i.e., therapeutically effective) amount of one or more (e.g. one) compounds of formula (I) to a patient in need of treatment, said compound of formula (I) being selected from the group consisting of: (IA) to (IH), (IJ) to (IL), 1 to 45, 1A to 45A, and 1B to 45B.

Another embodiment of this invention is directed to a method of treating

Alzheimer's disease, comprising administering an effective (i.e., therapeutically effective) amount of a compound of formula (I) to a patient in need of treatment, said compound of formula (I) being selected from the group consisting of: (IA) to (1H), (IJ) to (IL), 1 to 45, IA to 45A, and 1B to 45B.

This invention also provides combination therapies for (1) modulating gamma-secretase, or (2) treating one or more neurodegenerative diseases, or (3) inhibiting the deposition of amyloid protein (e.g., amyloid beta protein) in, on or around neurological tissue (e.g., the brain), or (4) treating Alzheimer's disease. The combination therapies are directed to methods comprising the administration of one or more (e.g. one) compounds of formula (IA) to (IH), (IJ) to (IL), 1 to 45, 1A to 45A, or 1B to 45B, and the administration of one or more (e.g., one) other pharmaceutical active ingredients (e.g., drugs). The compounds of formula (IA) to (IH), (IJ) to (IL), 1 to 45, 1A to 45A, or 1B to 45B, and the other drugs can be administered separately (i.e., each is in its own separate dosage form), or the compounds of formula (IA) to (IH), (IJ) to (IL), 1 to 45, 1A to 45A, or 1B to 45B can be combined with the other drugs in the same dosage form.

Thus, other embodiments of this invention are directed to any one of the methods of treatment, or methods of inhibiting, described herein, wherein the compounds of formulas (IA) to (IH), (IJ) to (IL), 1 to 45, IA to 45A, or 1B to 45B are used in combination with an effective amount of one or more other pharmaceutically active ingredients selected from the group consisting of: BACE inhibitors (beta secretase inhibitors), muscarinic antagonists (e.g., m₁ agonists or m₂ antagonists), cholinesterase inhibitors (e.g., acetyl- and/or butyrylchlolinesterase inhibitors); gamma secretase inhibitors; gamma secretase modulators; HMG-CoA reductase inhibitors; non-steroidal anti-inflammatory agents; N-methyl-D-aspartate receptor antagonists; anti-amyloid antibodies; vitamin E; nicotinic acetylcholine receptor agonists; CB1 receptor inverse agonists or CB1 receptor antagonists; an antibiotic; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors and cholesterol absorption inhibitors (e.g., ezetimibe).

Another embodiment of this invention is directed to a method of treating Alzheimer's disease, comprising administering an effective (i.e., therapeutically effective) amount of one or more (e.g., one) compounds of formula (IA) to (IH), (IJ) to (IL), 1 to 45, 1A to 45A, or 1B to 45B, in combination with an effective (i.e., therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of a compound of formula (IA) to (IH), (IJ) to (IL), 1 to 45, 1A to 45A, or 1B to 45B, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Alzheimer's disease, comprising administering an effective (i.e., therapeutically effective) amount of one or more (e.g., one) compounds of formula (IA) to (IH), (IJ) to (IL), 1 to 45, 1A to 45A, or 1B to 45B, in combination with an effective (i.e., therapeutically effective) amount of one or more compounds selected from the group consisting of Aβ antibody inhibitors, gamma secretase inhibitors and beta secretase inhibitors.

Another embodiment of this invention is directed to a method of treating Alzheimer's disease, comprising administering an effective (i.e., therapeutically effective) amount of one or more (e.g., one) compounds of formula (IA) to (IH), (IJ) to (IL), 1 to 45, 1A to 45A, or 1B to 45B, in combination with an effective (i.e., therapeutically effective) amount of one or more BACE inhibitors.

Another embodiment of this invention is directed to a method of treating Downs syndrome, comprising administering an effective (i.e., therapeutically effective) amount of one or more (e.g., one) compounds of formula (IA) to (IH), (IJ) to (IL), 1 to 45, 1A to 45A, or 1B to 45B to a patient in need of treatment.

Another embodiment of this invention is directed to a method of treating Downs syndrome, comprising administering an effective (i.e., therapeutically effective) amount of a compound of formula (IA) to (IH), (IJ) to (IL), 1 to 45, 1A to 45A, or 1B to 45B to a patient in need of treatment.

Another embodiment of this invention is directed to a method of treating Downs syndrome, comprising administering an effective (i.e., therapeutically effective) amount of one or more (e.g., one) compounds of formula (IA) to (IH), (IJ) to (IL), 1 to 45, 1A to 45A, or 1B to 45B, in combination with an effective therapeutically effective) amount of one or more cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to a method of treating Downs syndrome, comprising administering an effective (i.e., therapeutically effective) amount of a compound of formula (IA) to (IH), (IJ) to (IL), 1 to 45, 1A to 45A, or 1B to 45B, in combination with an effective (i.e., therapeutically effective) amount of one or more (e.g., one) cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), to a patient in need of treatment.

Another embodiment of this invention is directed to combinations (i.e., pharmaceutical compositions) comprising an effective (i.e., therapeutically effective) amount of one or more (e.g., one) compounds of formula (IA) to (IH), (IJ) to (IL), 1 to 45, 1A to 45A, or 1B to 45B, in combination with an effective (i.e., therapeutically effective) amount of one or more compounds selected from the group consisting of cholinesterase inhibitors (such as, for example, (±)-2,3-dihydro-5,6-dimethoxy-2-[[1-(phenylmethyl)-4-piperidinyl]methyl]-1H-inden-1-one hydrochloride, i.e., donepezil hydrochloride, available as the Aricept® brand of donepezil hydrochloride), Aβ antibody inhibitors, gamma secretase inhibitors and beta secretase inhibitors. The pharmaceutical compositions also comprise a pharmaceutically acceptable carrier.

This invention also provides a kit comprising, in separate containers, in a single package, pharmaceutical compositions for use in combination, wherein one container comprises an effective amount of a compound of formula (IA) to (IH), (IJ) to (IL), 1 to 45, 1A to 45A, or 1B to 45B in a pharmaceutically acceptable carrier, and another container (i.e., a second container) comprises an effective amount of another pharmaceutically active ingredient (as described above), the combined quantities of the compound of formula (I) and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

This invention also provides a kit comprising, in separate containers, in a single package, pharmaceutical compositions for use in combination, wherein one container comprises an effective amount of a compound selected from the group consisting of the compounds of formulas (IA) to (IH), (IJ) to (IL), 1 to 45, 1A to 45A, and 1B to 45B in a pharmaceutically acceptable carrier, and another container (i.e., a second container) comprises an effective amount of another pharmaceutically active ingredient (as described above), the combined quantities of the compounds of formulas (IA) to (IH), (IJ) to (IL), 1 to 45, 1A to 45A, or 1B to 45B and the other pharmaceutically active ingredient being effective to: (a) treat Alzheimer's disease, or (b) inhibit the deposition of amyloid protein (e.g., amyloid beta protein) in, on or around neurological tissue (e.g., the brain), or (c) treat neurodegenerative diseases, or (d) modulate the activity of gamma-secretase.

Other embodiments of this invention are directed to any one of the above embodiments wherein one or more (e.g., one) compounds selected from the group consisting of; A1, A2, and A3 are used instead of the compounds of formulas (IA) to (IH), (IJ) to (IL), 1 to 45, 1A to 45A, or 1B to 45B.

Examples of cholinesterase inhibitors are tacrine, donepezil, rivastigmine, galantamine, pyridostigmine and neostigmine, with tacrine, donepezil, rivastigmine and galantamine being preferred.

Examples of m₁ agonists are known in the art. Examples of m₂ antagonists are also known in the art; in particular, m₂ antagonists are disclosed in U.S. Pat. Nos. 5,883,096; 6,037,352; 5,889,006; 6,043,255; 5,952,349; 5,935,958; 6,066,636; 5,977,138; 6,294,554; 6,043,255; and 6,458,812; and in WO 03/031412, all of which are incorporated herein by reference.

Examples of BACE inhibitors include those described in: US2005/0119227 published Jun. 2, 2005 (see also WO2005/016876 published 02124/2005), US2005/0043290 published Feb. 24, 2005 (see also WO2005/014540 published Feb. 17, 2005), WO2005/058311 published Jun. 30, 2005 (see also US2007/0072852 published Mar. 29, 2007), US2006/0111370 published May 25, 2006 (see also WO2006/065277 published Jun. 22, 2006), U.S. application Ser. No. 11/710,582 filed Feb. 23, 2007, US2006/0040994 published Feb. 23, 2006 (see also WO2006/014762 published Feb. 9, 2006), WO2006/014944 published Feb. 9, 2006 (see also US2006/0040948 published Feb. 23, 2006), WO2006/138266 published Dec. 28, 2006 (see also US2007/0010667 published Jan. 11, 2007), WO2006/138265 published Dec. 28, 2006, WO2006/138230 published Dec. 28, 2006, WO2006/138195 published Dec. 28, 2006 (see also US2006/0281729 published Dec. 14, 2006), WO2006/138264 published Dec. 28, 2006 (see also US2007/0060575 published Mar. 15, 2007), WO2006/138192 published Dec. 28, 2006 (see also US2006/0281730 published Dec. 14, 2006), WO2006/138217 published Dec. 28, 2006 (see also US2006/0287294 published Dec. 21, 2006), US2007/0099898 published May 3, 200 (see also WO2007/050721 published May 3, 2007), WO2007/053506 published May 10, 2007 (see also US2007/099875 published May 3, 2007), U.S. application Ser. No. 11/759,336 filed Jun. 7, 2007, U.S. Application Ser. No. 60/874,362 filed Dec. 12, 2006, and U.S. Application Ser. No. 60/874,419 filed Dec. 12, 2006, the disclosures of each being incorporated incorporated herein by reference thereto.

It is noted that the carbons of formula (I) and other formulas herein may be replaced with 1 to 3 silicon atoms so long as all valency requirements are satisfied.

As used above, and throughout this disclosure, the following terms, unless otherwise indicated, shall be understood to have the following meanings:

“Patient” includes both human and animals.

“Mammal” means humans and other mammalian animals.

“One or more” means that there is at least one and there can be more than one, and examples include 1, 2 or 3, or 1 and 2, or 1.

“At least one” means there is at least one and there can be more than one, and examples include 1, 2 or 3, or 1 and 2, or 1.

“Bn” means benzyl.

“Et” means ethyl.

“i-pr” means isopropyl.

“Pr” means propyl.

“t-Bu” means tert-butyl.

“Fused benzocycloalkyl ring” means a phenyl ring fused to a cycloalkyl ring (as cycloalkyl is defined below), such as, for example,

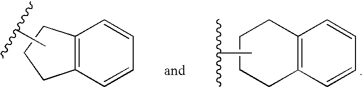

“Alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain, Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)— cycloalkyl, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl, aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain, Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain, “Lower alkynyl” means about 2 to about 6 carbon atoms in the chain which may be straight or branched, lore limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl- group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl- group in which the alkyl and aryl are as previously described. Preferred akylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyis include cyclopentenyl, cyclohexenyl, cyclohepta-1,3-dienyl, and the like. Non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halogen” means fluorine, chlorine, bromine, or iodine. Preferred are fluorine, chlorine and bromine. “Halo” refers to fluoro, chloro, bromo or iodo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, aryithic, heteroaryithio, aralkylthio, heteroaralkylthio, cycloalkyl, heterocyclyl, —O—C(O)-alkyl, —O—C(O)-aryl, —O—C(O)— cycloalkyl, —C(═N—CN)—NH₂, —C(═NH)—NH₂, —C(═NH)—NH(alkyl), oxime (e.g., ═N—OH), Y₁Y₂N—, Y₁Y₂N-alkyl-, Y₁Y₂NC(O)—, Y₁Y₂NSO₂— and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

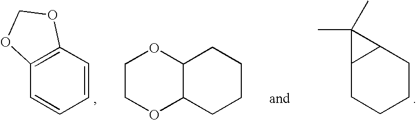

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” or “heterocycloalkyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention, The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like “Heterocyclyl” also includes rings wherein ═O replaces two available hydrogens on the same carbon atom on a ring system heterocyclyl includes rings having a carbonyl in the ring). An example of such moiety is pyrrolidone:

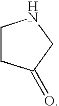

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide, S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyi, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes rings wherein ═O replaces two available hydrogens on the same carbon atom on a ring system (i.e., heterocyclyl includes rings having a carbonyl in the ring). An example of such moiety is pyrrolidinone:

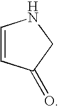

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

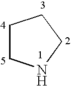

there is no —OH attached directly to carbons marked 2 and 5.

It should also be noted that tautomeric forms such as, for example, the moieties:

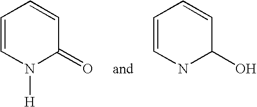

are considered equivalent in certain embodiments of this invention.

“Alkynylalkyl” means an alkynyl-alkyl- group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl- group in which the heteroaryl and alkyl are as previously described. Preferred heteroaralkyls contain a rower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl,

“Alkoxy” means an alkyl-O— group in which the alkyl group is as previously described. Nan-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—CO— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle, R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₉)alkylcarbamoyl-(C1-C2)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates, Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci. 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (Inner salts“) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formula (I), and salts, solvates, esters and prodrugs thereof, may exist in their tautomeric form (for example, as an amide, enol, keto or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention. In addition, the present invention embraces all geometric and positional isomers. For example, if a compound of Formula (I) incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds of Formula I, and of the salts, solvates, esters and prodrugs of the compounds of Formula I, are intended to be included in the present invention.

The compounds according to the invention can have pharmacological properties; in particular, the compounds of Formula I can be modulators of gamma secretase (including inhibitors, antagonists and the like).

More specifically, the compounds of Formula I can be useful in the treatment of a variety of disorders of the central nervous system including, for example, including, but not limited to, Alzheimer's disease, AIDS-related dementia, Parkinson's disease, amyotrophic lateral sclerosis, retinitis pigmentosa, spinal muscular atrophy and cerebellar degeneration and the like.

Another aspect of this invention is a method of treating a mammal (e.g., human) having a disease or condition of the central nervous system by administering a therapeutically effective amount of at least one compound of Formula I, or a pharmaceutically acceptable salt, solvate, ester or prodrug of said compound to the mammal.

A preferred dosage is about 0.001 to 500 mg/kg of body weight/day of the compound of Formula I. An especially preferred dosage is about 0.01 to 25 mg/kg of body weight/day of a compound of Formula I, or a pharmaceutically acceptable salt or solvate of said compound.

The compounds of this invention may also be useful in combination (administered together or sequentially) with one or more additional agents listed above.

The compounds of this invention may also be useful in combination (administered together or sequentially) with one or more compounds selected from the group consisting of Aβ antibody inhibitors, gamma secretase inhibitors and beta secretase inhibitors.

If formulated as a fixed dose, such combination products employ the compounds of this invention within the dosage range described herein and the other pharmaceutically active agent or treatment within its dosage range.

Accordingly, in an aspect, this invention includes combinations comprising an amount of at least one compound of Formula I, or a pharmaceutically acceptable salt, solvate, ester or prodrug thereof, and an amount of one or more additional agents listed above wherein the amounts of the compounds/treatments result in desired therapeutic effect.

The pharmacological properties of the compounds of this invention may be confirmed by a number of pharmacological assays. Certain assays are exemplified later in this document.

This invention is also directed to pharmaceutical compositions which comprise at least one compound of Formula I, or a pharmaceutically acceptable salt, solvate, ester or prodrug of said compound and at least one pharmaceutically acceptable carrier.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula (I), or a pharmaceutically acceptable salt, solvate, ester or prodrug of said compound and a pharmaceutically acceptable carrier, vehicle or diluent.

Yet another aspect of this invention is a kit comprising an amount of at least one compound of Formula (I), or a pharmaceutically acceptable salt, solvate, ester or prodrug of said compound and an amount of at least one additional agent listed above, wherein the amounts of the two or more ingredients result in desired therapeutic effect.

The invention disclosed herein is exemplified by the following illustrative schemes and examples which should not be construed to limit the scope of the disclosure. Alternative mechanistic pathways and analogous structures will be apparent to those skilled in the art.

The compounds of the invention can be prepared by the schemes and examples below.

### Example 1

### Compounds A1 and A2

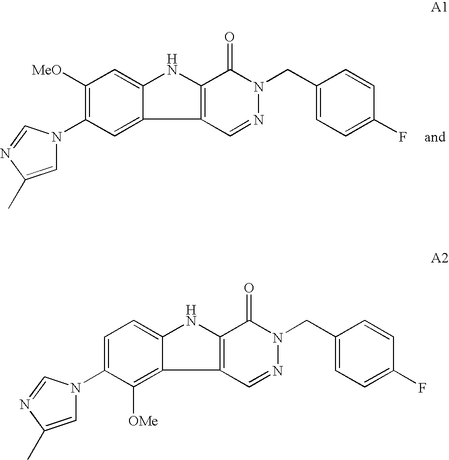

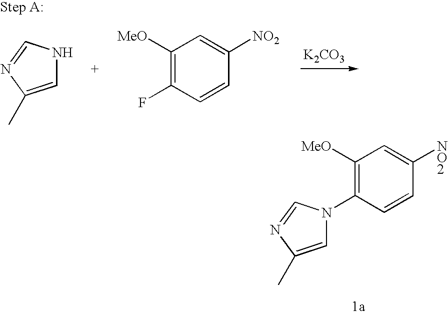

4-methylimidazole (2.0 mmol), 3-methoxy-4-fluoro-nitrobenzene (1.0 mmol) and K₂CO₃ (5 mmol) were stirred in CH₃CN (10 mL) at room temperature over night. The reaction mixture was filtered and concentrated under reduced pressure. The crude product was recrystallized with EtOAc to give desired product 1a.

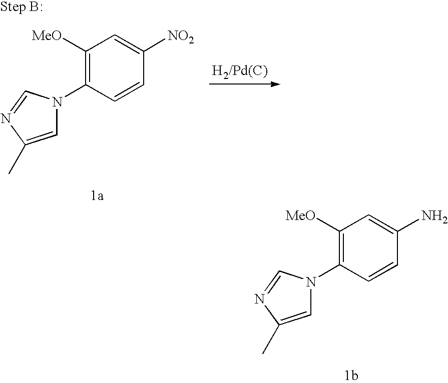

Compound 1a was hydrogenated with hydrogen balloon in the presence of Pd(C) as the catalyst (10 wt %) in MeOH over night. The mixture was filtered and concentrated under reduced pressure to give product 1b.

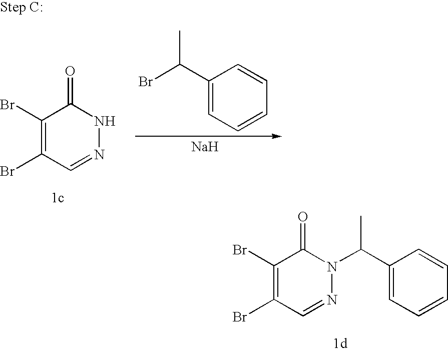

NaH (0.397 g, 9.06 mmol, 1.15 eq) was added to a solution of compound 1c (2.0 g, 7.88 mmol, 1 eq) in DMF (10 mL) at 0° C. The mixture was stirred for 15 minutes before alpha-methyl benzyl bromide (1.34 mL, 9.85 mmol, 1.25 eq) was added dropwise. The resulting reaction mixture was stirred at room temperature over night. The mixture was diluted with EtOAc (200 mL) and HCl solution (30 mL, 0.5 M). The organic layer was washed with water (3×), brine, dried over MgSO₄, and concentrated to give the crude product. The crude residue was purified by column chromatography eluting with EtOAc/hexanes to yield compound 1d (2.43 g, 86%).

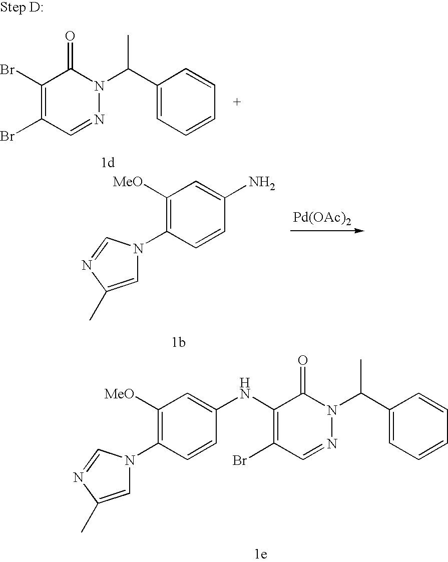

A mixture of compound 1d (0.118 mmol), 1b (24 mg, 0.118 mmol), Pd(OAc)₂ (1.06 mg, 0.00472 mmol), BINAP (2.94 mg, 0.00472 mmol) and K₂CO₃ (81.4 mg, 0.59 mmol) in toluene was vacuum/nitrogen exchange degassed for 3 times before it was heated at 120° C. for 48 hours. The reaction mixture was cooled and diluted with EtOAc (50 mL) and NH₄Cl solution (10 mL). The organic layer was washed with water, brine, dried over MgSO₄, and concentrated to give the crude product. The crude residue was purified by Gilson reverse phase HPLC to yield Compound 1e.

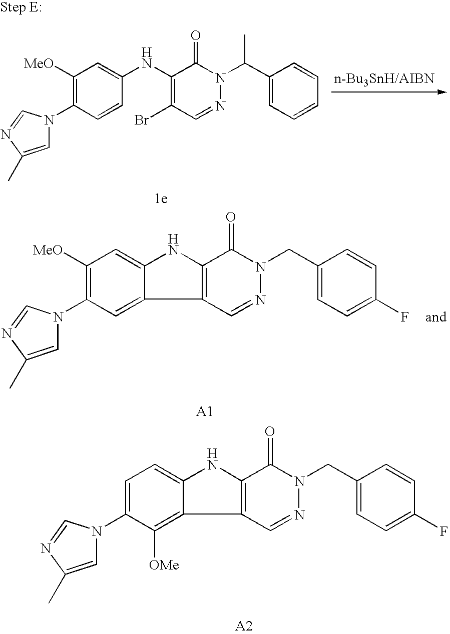

A mixture of compound 1e (30 mg. 0.0619 mmol), n-Bu₃SnH (31.8 uL, 0.12 mmol), AIBN (1.84 mg, 0.00112 mmol) in benzene (2 mL) was vacuum/nitrogen exchange degassed for 3 times before it was heated at 100° C. for 24 hours. The reaction mixture was cooled and solvent was removed under vacuum. The crude product was purified by Gilson reverse phase HPLC to yield Compound A1, Electrospray MS [M+1]⁺ 404.2; and Compound A2, Electrospray MS [M+1]⁺ 404.2

### Example 2

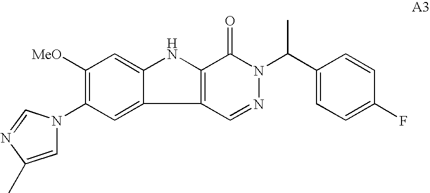

Compound A3 was prepared following essentially the same procedure as Example 1, Electrospray MS [M+1]⁺ 418.2.

### Assay:

Secretase Reaction and Aβ Analysis in Whole Cells; HEK293 cells overexpressing APP with Swedish and London mutations are treated with the specified compounds for 5 hour at 37° C. in 100 ml of DMEM medium containing 10% fetal bovine serum. At the end of the incubation, total Aβ, Aβ40 and Aβ42 are measured using electrochemiluminescence (ECL) based sandwich immunoassays. Total Aβ is determined using a pair of antibodies TAG-W02 and biotin-4G8, Aβ40 is identified with antibody pairs TAG-G2-10 and biotin-4G8, while Aβ42 is identified with TAG-G2-11 and biotin-4G8. The ECL signal is measured using Sector Imager 2400 (Meso Scale Discovery).

MS Analysis of Aβ Profile: Aβ profile in conditioned media is determined using surface enhanced laser desorption/ionization (SELDI) mass spectrometry. Conditioned media is incubated with antibody W02 coated PS20 ProteinChip array. Mass spectra of Aβ captured on the array are read on SELDI ProteinChip Reader (Bio-Rad) according to manufacture's instructions.

CSF Aβ Analysis: Aβ in rat CSF is determined using. MSD technology as described above. Aβ40 is measured using antibody pair Tag-G2-10 and biotin-4G8, while Aβ42 is measured using Tag-anti Aβ42 (Meso Scale Discovery) and biotin-4G8. The ECL signal is measured using Sector Imager 2400 (Mesa Scale Discovery).

Matrix-assisted laser desorption/ionization mass spectrometric (MALDI MS) analysis of Aβ is performed on a Voyager-DE STR mass spectrometer (ABI, Framingham, Mass.). The instrument is equipped with a pulsed nitrogen laser (337 nm). Mass spectra are acquired in the linear mode with an acceleration voltage of 20 kV. Each spectrum present in this work represents an average of 256 laser shots. To prepare the sample-matrix solution, 1 μL of immunoprecipitated Aβ sample is mixed with 3 μL of saturated α-cyano-4-hydroxycinnamic acid solution in 0.1% TFA/acetonitrile. The sample-matrix solution is then applied to the sample plate and dried at ambient temperature prior to mass spectrometric analysis. All the spectra are externally calibrated with a mixture of bovine insulin and ACTH (18-39 clip).

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A compound of the formula (I): 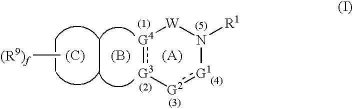
or a pharmaceutically acceptable salt, ester, or solvate thereof, wherein:
Ring (B) is a 4 to 10 membered single ring or fused ring selected from the group consisting of: cycloalkyl, cycloalkenyl, aryl, heteroaryl, heterocycloalkyl, and heterocycloalkenyl, and
(a) said heterocycloalkyl Ring (B), or said heterocycloalkenyl Ring (B), or said heteroaryl Ring (B), comprises 1 to 4 heteroatoms (including any heteroatoms common to Ring (A) and Ring (C)) selected from the group consisting of —NR²—, —O—, —S—, —S(O)— and —S(O)₂, and
(b) Ring (B) is optionally substituted with 1 to 5 substitutents independently selected from the group consisting of: ═O, ═NR² and R²¹;
Ring (C) is an aryl or heteroaryl ring, and said Ring (C) is optionally substituted with 1 to 3 independently selected R²¹ substitutents;
the dotted line ( [symbol] ) between positions (1) and (2) represents an optional bond;
the dotted line ( [symbol] ) between positions (3) and (4) represents an optional bond;
d is 0 or 1;
f is 1;
m is 0 to 6;
n is 1 to 5;
p is 0 to 5;
q is 0, 1 or 2, and each q is independently selected;
W is selected from the group consisting of: —C(O)—, —S(O)₂—, —S(O)—, and —C(═NR²)—;
G¹ is selected from the group consisting of: a direct bond (i.e., the N at (5) is bonded directly to G², and Ring A is a five membered ring), —O—, —C(R²¹)_{q}, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q} group is 0 or 1, and
(b) d for the —N(R²)_{d}— group is 0; and
(c) G¹ is not —O—, —C(O)—, —C(═NR²)—, —S—, —S(O)₂, or S(O)—;
G² is selected from the group consisting of: a direct bond, —O—, —C(R²¹)_{q}, —N(R²)_{d}—, —C(O)—, —C(═NR²)—, —S—, —S(O)₂, and —S(O)—; and with the proviso that when the optional double bond between (3) and (4) is present then:
(a) q for the —C(R²¹)_{q} group is 0 or 1, and
(b) d for the —N(R²)_{d}— group is 0; and
(c) G² is not —O—, —C(O)—, —C(═NR²)—, —S—, —S(O)₂, or —S(O)—;
G³ is:
(a) carbon when the optional bond between G³ and G⁴ is present,
(b) carbon when the optional bond between G³ and G⁴ is absent, and there is a double bond to G³ in Ring (B), and
(c) carbon or nitrogen when the optional bond between G³ and G⁴ is absent, and there is single double bond to G³ in Ring (B);
G⁴ is:
(a) carbon when the optional bond between G³ and G⁴ is present,
(b) carbon when the optional bond between G³ and G⁴ is absent, and there is a double bond to G⁴ in Ring (B), and
(c) carbon or nitrogen when the optional bond between G³ and G⁴ is absent, and there is single double bond to G⁴ in Ring (B);
R¹ is selected from the group consisting of: alkyl-, alkenyl-, alkynyl-, aryl-, arylalkyl-, alkylaryl-, cycloalkyl-, cycloalkenyl, cycloalkylalkyl-, fused benzocycloalkyl, fused benzoheterocycloalkyl, fused heteroarylcycloalkyl, fused heteroarylheterocycloalkyl, heteroaryl-, heteroarylalkyl-, heterocyclyl-, heterocyclenyl, -and heterocyclyalkyl-; wherein each of said alkyl-, alkenyl- and alkynyl-, aryl-, arylalkyl-, alkylaryl-, cycloalkyl-, cycloalkenyl-, cycloalkylalkyl-, fused benzocycloalkyl, fused benzaheterocycloalkyl, fused heteroarylcycloalkyl, fused heteroarylheterocycloalkyl, heteroaryl-, heteroarylalkyl-, heterocyclyl-, heterocyclenyl and heterocyclyalkyl-R¹ groups is optionally substituted with 1-5 independently selected R²¹ groups; or
R² is selected from the group consisting of: H, —OH, —O-alkyl, —O-(halo substituted alky), —NH(R⁴), —N(R⁴)₂ (wherein each R⁴ is independently selected), —NH₂, —S(R⁴), —S(O)R⁴, —S(O)(OR⁴), —S(O)₂1R⁴—S(O)₂(OR⁴), —S(O)NHR⁴, —S(O)N(R⁴)₂, —S(O)NH₂, —S(O)₂NHR⁴, —S(O)₂N(R⁴)₂, —S(O)₂NH₂, —CN, —C(O)₂R⁴, —C(O)NHR⁴, —C(O)N(R⁴)₂, —C(O)NH₂, —C(O)R⁴, unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
Each R⁴ is independently selected from the group consisting of: unsubstituted aryl, substituted aryl, unsubstituted heteroaryl, substituted heteroaryl, unsubstituted alkyl, substituted alkyl, unsubstituted arylalkyl-, substituted arylalkyl-, unsubstituted heteroarylalkyl-, substituted heteroarylalkyl-, unsubstituted alkenyl, substituted alkenyl, unsubstituted alkynyl, substituted alkynyl, unsubstituted cycloalkyl, and substituted cycloalkyl, wherein said substituted aryl, heteroaryl, alkyl, arylalkyl-, heteroarylalkyl-, alkenyl, alkynyl and cycloalkyl groups are substituted with 1 to 5 independently selected R²¹ groups;
R⁹ is selected from the group consisting of: aryl-, arylalkyl-, cycloalkyl-, cycloalkenyl, cycloalkylalkyl-, heteroaryl-, heteroarylalkyl-, heterocyclyl-, heterocyclenyl-, and heterocyclyalkyl-, wherein each of said R⁹ aryl-, arylalkyl-, cycloalkyl-, cycloalkenyl, cycloalkylalkyl-, heteroaryl-, heteroarylalkyl-, heterocyclyl-, heterocyclenyl-, heterocyclyalkyl- and heterocyclyalkyl- is optionally substituted with 1-3 independently selected R²¹ groups;
R¹⁴ is selected from the group consisting of H, alkyl, alkenyl, alkynyl, cycloalkyl, cycloalkylalkyl, cycloalkenyl, heterocyclyl, heterocyclenyl, heterocyclylalkyl, heterocyclyalkenyl-, aryl, arylalkyl, heteroaryl, heteroarylalkyl, —CN, —C(O)R¹⁵, —C(O)OR¹⁵, —C(O)N(R¹⁵)(R¹⁶), —S(O)N(R¹⁵)(R¹⁶), —S(O)₂N(R¹⁵)(R¹⁶), —C(═NO¹⁵)R¹⁶, and —P(O)(OR¹⁵)(OR¹⁶);
R¹⁵, R¹⁶ and R¹⁷ are independently selected from the group consisting of H, alkyl, alkenyl, alkynyl, cycloalkyl, cycloalkylalkyl, heterocyclyl, heterocyclylalkyl, aryl, arylalkyl, heteroaryl, heteroarylalkyl, arylcycloalkyl, arylheterocyclyl, (R¹⁸)-alkyl, (R¹⁸)ₙ-cycloalkyl, (R¹⁸)ₙ-cycloalkylalkyl, (R¹⁸)ₙ-heterocyclyl, (R¹⁸)ₙ-heterocyclylalkyl, (R¹⁸)ₙ-aryl, (R¹⁸)ₙ-arylalkyl, (R¹⁸)ₙ-heteroaryl and (R¹⁸)ₙ-heteroarylalkyl; or
R¹⁵, R¹⁶ and R¹⁷ are independently selected from the group consisting of: 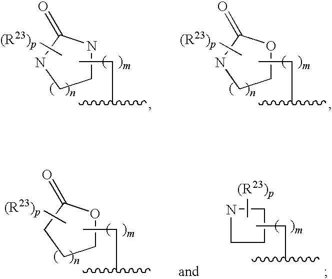
Each R¹⁸ is independently selected from the group consisting of alkyl, alkenyl, alkynyl, aryl, arylalkyl, arylalkenyl, arylalkynyl, —NO₂, halo, heteroaryl, HO-alkyoxyalkyl, —CF₃, —CN, alkyl-CN, —C(O)R¹⁹, —C(O)OH, —C(O)OR¹⁹, —C(O)NHR²⁰, —C(O)NH₂, —C(O)NH₂—C(O)N(alkyl)₂, —C(O)N(alkyl)(aryl), —C(O)N(alkyl)(heteroaryl), —SR¹⁹, —S(O)₂R²⁰, —S(O)NH₂, —S(O)NH(alkyl), —S(O)N(alkyl)(alkyl), —S(O)NH(aryl), —S(O)₂NH₂, —S(O)₂NHR¹⁹, —S(O)₂NH(heterocyclyl), —S(O)₂N(alkyl)₂, —S(O)₂N(alkyl)(aryl), —OCF₃, —OH, —OR²⁰, —O-heterocyclyl, —O-cycloalkylalkyl, —O-heterocyclylalkyl, —NH₂, —NHR²⁰, —N(alkyl)₂, —N(arylalkyl)₂, —N(arylalkyl)-(heteroarylalkyl), —NHC(O)R²⁰, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁰, —NHS(O)₂NH(alkyl), —NHS(O)₂N(alkyl)(alkyl), —N(alkyl)S(O)₂NH(alkyl) and —N(alkyl)S(O)₂N(alkyl)(alkyl); or
two R¹⁸ moieties on adjacent carbons can be linked together to form a 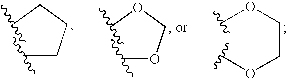
R¹⁹ is selected from the group consisting of: alkyl, cycloalkyl, aryl, arylalkyl and heteroarylalkyl;
R²⁰ is selected from the group consisting of: alkyl, cycloalkyl, aryl, halo substituted aryl, arylalkyl, heteroaryl and heteroarylalkyl;
Each R²¹ is independently selected from the group consisting of: alkyl, alkenyl, alkynyl, cycloalkyll, cycloalkylalkyl, cycloalkenyl, heterocycloalkyl, heterocycloalkylalkyl, aryl, arylalkyl, heteroaryl, heteroarylalky, halo, —ON, —OR¹⁵,
—C(O)R¹⁵, —C(O)OR¹⁵, —O(O)N(R¹⁵)(R¹⁶), —SR¹⁵, —S(O)N(R¹⁵)(R¹⁶), —CH(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —CH₂—N(R¹⁵)C(O)N(R¹⁶)(R¹⁷), —CH₂—R¹⁵; —CH₂N(R¹⁵)(R¹⁶), —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —S(O)R¹⁵, ═NOR¹⁵, —N₃, —NO₂, —S(O)₂R¹⁵, —O—N═C(R⁴)₂ (wherein each R⁴ is independently selected), and —O—N═C(R⁴)₂ wherein R⁴ is taken together with the carbon atom to which they are bound to form a 5 to 10 membered ring, said ring optionally containing 1 to 3 heteroatoms selected from the group consisting of —O—, —S—, —S(O)—, —S(O)₂—, and —NR²—; wherein each of said alkyl, alkenyl, alkynyl, cycloalkyl, cycloalkylalkyl, cycloalkenyl, heterocycloalkyl, heterocycloalkylalkyl, aryl, arylalkyl, heteroaryl, and heteroarylalkyl R²¹ groups is optionally substituted with 1 to 5 independently selected R²² groups;
Each R group is independently selected from the group consisting of alkyl, cycloalkyl, cycloalkenyl, heterocycloalkyl, aryl, heteroaryl, halo, —CF₃, —CN, —OR¹⁵, —C(O)R¹⁵, —C(O)OR¹⁵, -alkyl-C(O)OR¹⁵, C(O)N(R¹⁵)(R¹⁶), —SR¹⁵, —S(O)N(R¹⁵)(R¹⁶), —S(O)₂N(R¹⁵)(R¹⁶), —C(═NOR¹⁵)R¹⁶, —P(O)(OR¹⁵)(OR¹⁶), —N(R¹⁵)(R¹⁶), -alkyl-N(R¹⁵)(R¹⁶), —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)OR¹⁶, —CH₂—N(R¹⁵)C(O)OR¹⁶, —N₃, ═NOR¹⁵, —NO₂, —S(O)R¹⁵ and —S(O)₂R¹⁵;
or two R²¹ or two R²² moieties on adjacent carbons can be linked together to form a 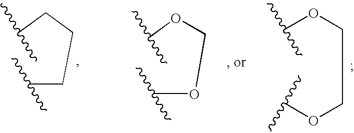
and when R²¹ or R²² is selected from the group consisting of —C(═NOR¹⁵)R¹⁶, —N(R¹⁵)C(O)R¹⁶, —CH₂—N(R¹⁵)C(O)R¹⁶, —N(R¹⁵)S(O)R¹⁶, —N(R¹⁵)S(O)₂R¹⁶, —CH₂—N(R¹⁵)S(O)₂R¹⁶, —N(R¹⁵)S(O)₂N(R¹⁶)(R¹⁷), —N(R¹⁵)S(O)N(R¹⁶)(R¹⁷), —N(R¹⁵)C(O)N(R¹⁶)(R¹⁷), —CH₂—N(R¹⁵)C(O)N(R¹⁶)(R¹⁷), —N(R¹⁵)C(O)R¹⁶ and —CH₂—N(R¹⁵)C(O)OR¹⁶, then R¹⁵ and R¹⁶ together can form a C₂ to C₄ chain wherein, optionally, one, two or three ring carbons can be replaced by —C(O)— or —N(H)— and R¹⁵ and R¹⁸, together with the atoms to which they are attached, form a 5 to 7 membered ring, optionally substituted by (R²³)ₙ;
Each R²³ is independently selected from the group consisting of alkyl, alkenyl, alkynyl, cycloalkyl, cycloalkylalkyl, cycloalkenyl, heterocycloalkyl, heterocycloalkylalkyl, aryl, arylalkyl, heteroaryl, heteroarylalkyl, halo, —CN, —OR²⁴, —C(O)R²⁴, —C(O)OR²⁴, —C(O)N(R²⁴)(R²⁵), —SR²⁴, —S(O)N(R²⁴)(R²⁵), —S(O)₂N(R²⁴)(R²⁵), —C(═NOR²⁴)R²⁵, —P(O)(OR²⁴)(OR²⁵), —N(R²⁴)(R²⁵), -alkyl-N(R²⁴)(R²⁵), —N(R²⁴)C(O)R²⁵, —CH₂—N(R²⁴)C(O)R²⁵, —N(R²⁴)S(O)R²⁵, —N(R²⁴)S(O)₂R²⁵, —CH₂—N(R²⁴)S(O)₂R²⁵, —N(R²⁴)S(O)₂N(R²⁵)(R²⁶), —N(R²⁴)S(O)N(R²⁵)(R²⁶), —N(R²⁴)C(O)N(R²⁵)(R²⁶), —CH₂—N(R²⁴)C(O)N(R²⁵)(R²⁶), —N(R²⁴)C(O)OR²⁵, —CH₂—N(R²⁴)C(O)OR²⁵, —S(O)R²⁴ and —S(O)₂R²⁴; and wherein each of the alkyl, cycloalkyl, cycloalkylalkyl, heterocycloalkyl, heterocycloalkylalkyl, aryl, arylalkyl, heteroaryl, heteroarylalkyl, alkenyl and alkynyl R²³ groups is optionally substituted with 1 to 5 independently selected R²⁷ groups;
Each R²⁴, R²⁵ and R²⁶ is independently selected from the group consisting of H, alkyl, cycloalkyl, cycloalkylalkyl, heterocycloalkyl, heterocycloalkylalkyl, aryl, arylalkyl, heteroaryl, heteroarylalkyl, arylcycloalkyl, (R^{27A})ₙ-alkyl, (R^{27A})ₙ-cycloalkyl, (R^{27A})ₙ-cycloalkylalkyl, (R^{27A})ₙ-heterocycloalkyl, (R^{27A})ₙ-heterocycloalkylalkyl, (R^{27A})ₙ-aryl, (R^{27A})ₙ-arylalkyl, (R^{27A})ₙ-heteroaryl and (R^{27A})ₙ-heteroarylalkyl;
Each R²⁷ is independently selected from the group consisting of alkyl, cycloalkyl, heterocycloalkyl, aryl, heteroaryl, halo, —CF₃, —CN, —OR²⁴, —C(O)R²⁴, —C(O)OR²⁴, alkyl-C(O)OR²⁴, C(O)N(R²⁴)(R²⁵), —SR²⁴, —S(O)N(R²⁴)(R²⁵), —S(O)₂N(R²⁴)(R²⁵), —C(═NOR²⁴)R²⁵, —P(O)(OR²⁴)(OR²⁵), —N(R²⁴)(R²⁵), -alkyl-N(R²⁴)(R²⁵), —N(R²⁴)C(O)R²⁵, —CH₂—N(R²⁴)C(O)R²⁵, —N(R²⁴)S(O)R²⁵, —N(R²⁴)S(O)₂R²⁵, —CH₂—N(R²⁴)S(O)₂R²⁵, —N(R²⁴)S(O)₂N(R²⁵)(R²⁶), —N(R²⁴)S(O)N(R²⁵)(R²⁶), —N(R²⁴)C(O)N(R²⁵)(R²⁸), —CH₂—N(R²⁴)C(O)N(R²⁵)(R²⁶), —N(R²⁴)C(O)OR²⁵, —CH₂—N(R²⁴)C(O)OR²⁵, —S(O)R²⁴ and —S(O)₂R²⁴;
Each R^{27A} is independently selected from the group consisting of alkyl, aryl, arylalkyl, —NO₂, halo, —CF₃, —ON, alkyl-CN, —C(O)R²⁸, —C(O)OH, —C(O)OR²⁸, —C(O)NHR²⁹, —C(O)N(alkyl)₂, —C(O)N(alkyl)(aryl), —C(O)N(alkyl)(heteroaryl), —SR²⁸, —S(O)₂R²⁸, —S(O)NH₂, —S(O)NH(alkyl), —S(O)N(alkyl)(alkyl), —S(O)NH(aryl), —S(O)₂NH₂, —S(O)₂NHR²⁸, —S(O)₂NH(aryl), —S(O)₂NH(heterocycloalkyl), —S(O)₂N(alkyl)₂, —S(O)₂N(alkyl)(aryl), —OH, —OR²⁹, —O-heterocycloalkyl, —O-cycloalkylalkyl, —O-heterocycloalkylalkyl, —NH₂, —NHR²⁹, —N(alkyl)₂, —N(arylalkyl)₂, —N(arylalkyl)(heteroarylakyl), —NHC(O)R²⁹, —NHC(O)NH₂, —NHC(O)NH(alkyl), —NHC(O)N(alkyl)(alkyl), —N(alkyl)C(O)NH(alkyl), —N(alkyl)C(O)N(alkyl)(alkyl), —NHS(O)₂R²⁹, —NHS(O)₂NH(alkyl), —NHS(O)₂N(alkyl)(alkyl), —N(alkyl)S(O)₂NH(alkyl) and —N(alkyl)S(O)₂N(alkyl)(alkyl);
R²⁸ is selected from the group consisting of: alkyl, cycloalkyl, arylalkyl and heteroarylalkyl; and
R²⁹ is selected from the group consisting of; alkyl, cycloalkyl, aryl, arylalkyl, heteroaryl or heteroarylalkyl; and
provided that:
(a) Ring A does not have two adjacent —O— atoms in the ring; and
(b) Ring A does not have two adjacent sulfur groups in the ring (i.e., when there is a —S—, —S(O)— or —S(O)₂ group at one position in Ring A, then the adjacent positions in Ring A are not —S—, —S(O)— or —S(O)₂); and
(c) Ring A does not have an —O— atom adjacent to a sulfur group (i.e., Ring A does not have an —O— atom adjacent to a —S—, —S(O)— or —S(O)₂); and
(d) When G¹ is N, then G² is not —O—; and
(e) When G¹ is —O—, then G² is not N; and
(f) When G¹ is N, then G² is not —S—; and
(g) When G¹ is —S—, then G² is not N; and
(h) When G¹ is a direct bond, and G² is —O—, then G³ is not N; and
(i) When G² is a direct bond, and G¹ is —O—, then G³ is not N; and
(j) When G¹ is N, and G³ is N, then G² is not N; and
(k) When G² is N, and G³ is N, then G¹ is not N; and
(l) When G¹ is N, and G² is N, then G³ is not N.

2. The compound of claim 1 wherein Ring B is selected from the group consisting of: cyclopentyl, cyclohexyl, cycloheptyl, 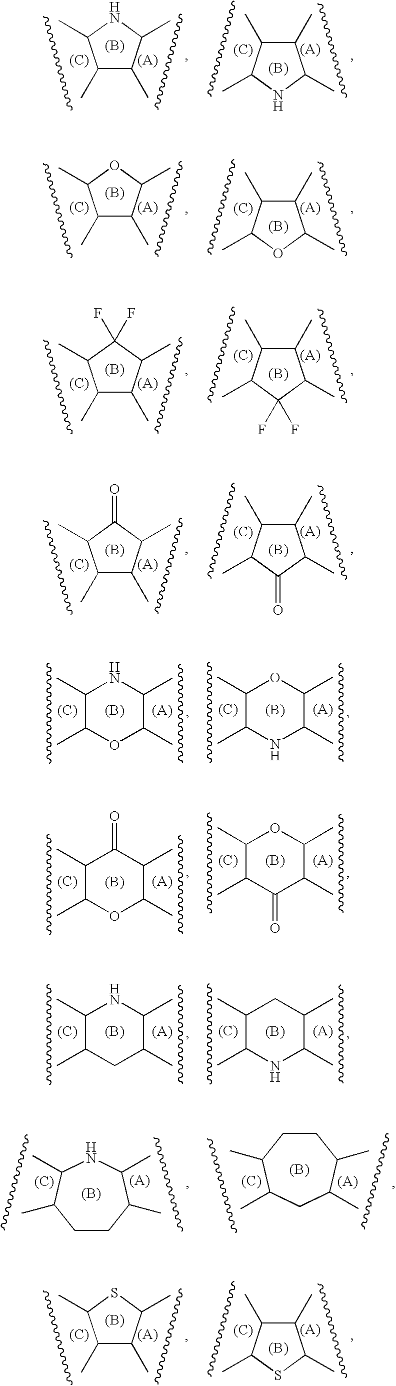 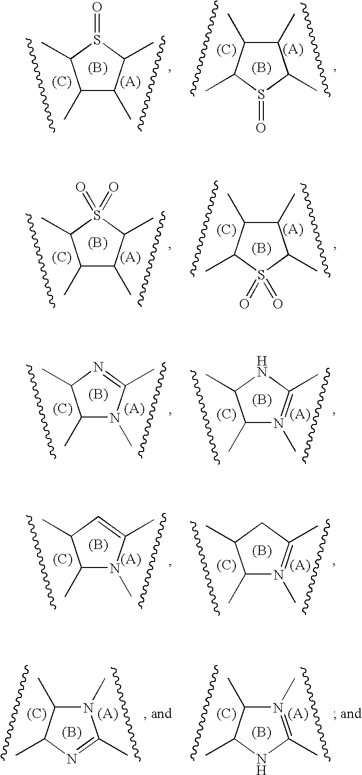
Ring (C) is selected from the group consisting of: 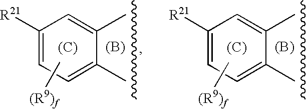
wherein R²¹ is —OR¹⁵, 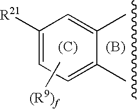
wherein R²¹ is —OR¹⁵ and R¹⁵ is alkyl, 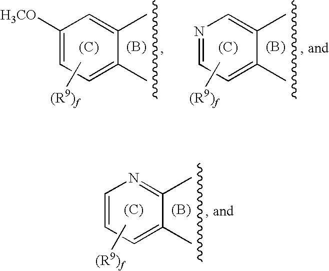
said R⁹ group is selected from the group consisting of heteroaryl and heteroaryl substituted with one or more R²¹ groups, wherein each R²¹ is independently selected.

3. The compound of claim 1 wherein said R⁹ is imidazolyl substituted with one R²¹ group, wherein each R²¹ is independently selected.

4. The compound of claim 1 wherein the R⁹—R(C)— moiety is selected from the group consisting of: 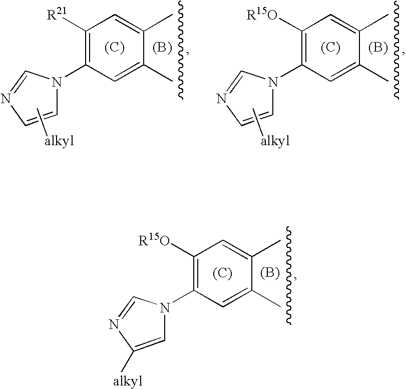
wherein said R¹⁵ is alkyl, 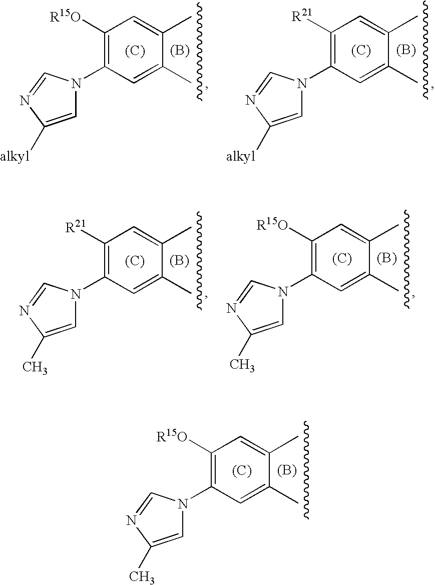
wherein said R¹⁵ is alkyl, 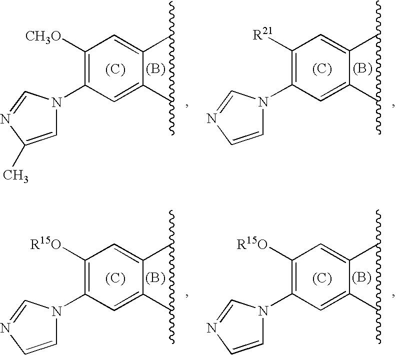
wherein said R¹⁵ is alkyl, and

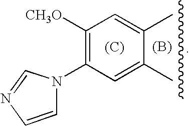

5. The compound of claim 1 wherein the R⁹-Ring (C)— moiety is:

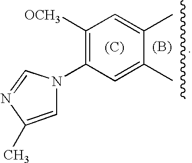

6. The compound of claim 1 wherein said R¹ group is: 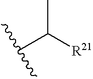
wherein R²¹ is unsubstituted or substituted with one or more independently selected R²² groups.

7. The compound of claim 1 wherein:
R¹ is an alkyl group substituted with one R²¹ group, and said R²¹ group is an aryl group; or
R¹ is an alkyl group substituted with one R²¹ group, and said R²¹ group is an aryl group, and said aryl is phenyl, and said alkyl group is methyl or ethyl; or
R¹ is an alkyl group substituted with one R²¹ group, and said R²¹ group is an aryl group, and said aryl group is substituted with one or more R²² groups; or
R¹ is an alkyl group substituted with one R²¹ group, and said R²¹ group is an aryl group, and said aryl group is substituted with one or more R²² groups wherein each R²² group is the same or different halo; or
R¹ is an alkyl group substituted with one R²¹ group, and said R²¹ group is an aryl group, and said aryl group is substituted with one or two R²² halo groups; or
R¹ is an alkyl group substituted with one R²¹ group, and said R²¹ group is an aryl group, and said aryl group is substituted with one or two R²² halo groups wherein the halo is F.

8. The compound of claim 1 wherein said R¹ is selected from the group consisting of:

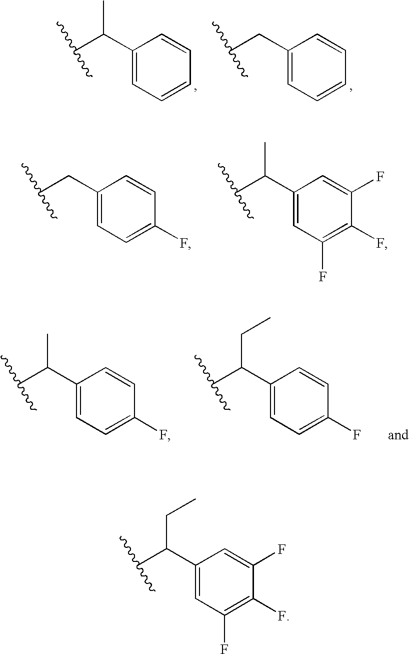

9. The compound of claim 1 wherein said R⁹ group is selected from the group consisting of heteroaryl and heteroaryl substituted with one or more R²¹ groups, and wherein each R²¹ is independently selected.

10. The compound of claim 4 wherein:
(1)
R¹ is an alkyl group substituted with one R²¹ group, or
R¹ is an alkyl group substituted with one R²¹ group, and said R²¹ group is substituted with one or more independently selected R²² groups, or
(2)
R¹ is an alkyl group substituted with one phenyl, or
R¹ is an alkyl group substituted with one phenyl, and said phenyl is substituted with one or more independently selected R²² groups, or
(3)
R¹ is a methyl or ethyl group substituted with one phenyl, or
R¹ is a methyl or ethyl group substituted with one phenyl, and said phenyl is substituted with one or more independently selected halos, or
(4)
R¹ is a methyl or ethyl group substituted with one phenyl, or
R¹ is an methyl or ethyl group substituted with one phenyl, and said phenyl is substituted with one or two independently selected halos, or
(5)
R¹ is a methyl or ethyl group substituted with one phenyl, or
R¹ is an methyl or ethyl group substituted with one phenyl, and said phenyl is substituted with one or two F, or
(6)
R¹ is a methyl or ethyl group substituted with one phenyl, or
R¹ is an methyl or ethyl group substituted with one phenyl, and said phenyl is substituted with one or two F, or
(7)
R¹ is selected from the group consisting of: 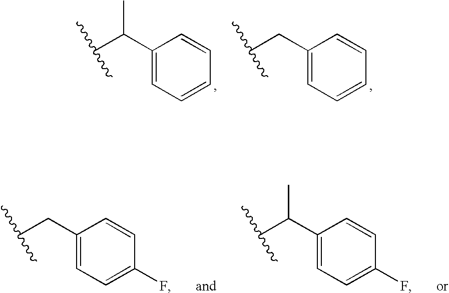
(8)
R¹ is selected from the group consisting of: 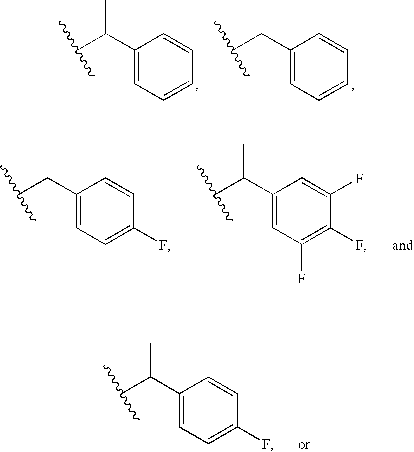
(9)
R¹ is selected from the group consisting of: 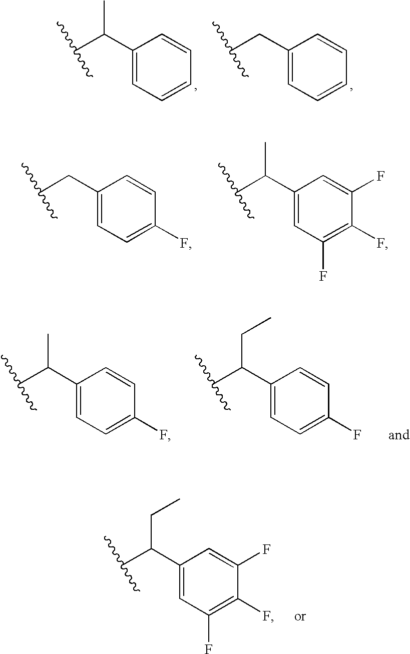
(10)
R¹ is selected from the group consisting of:

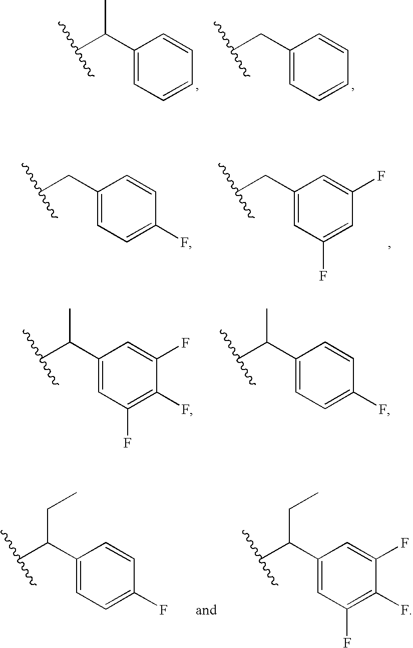

11. The compound of claim 10 wherein W is —C(O)—.

12. The compound of claim 1 selected from the group consisting of having the formula:

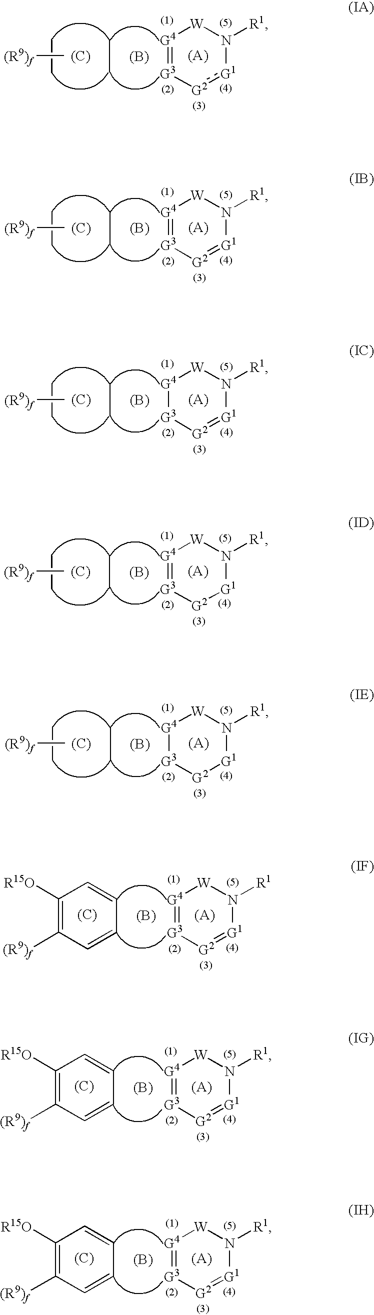

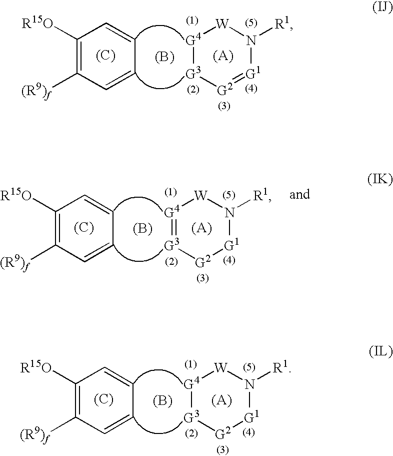

13. The compound of claim 1 selected from the group consisting of:

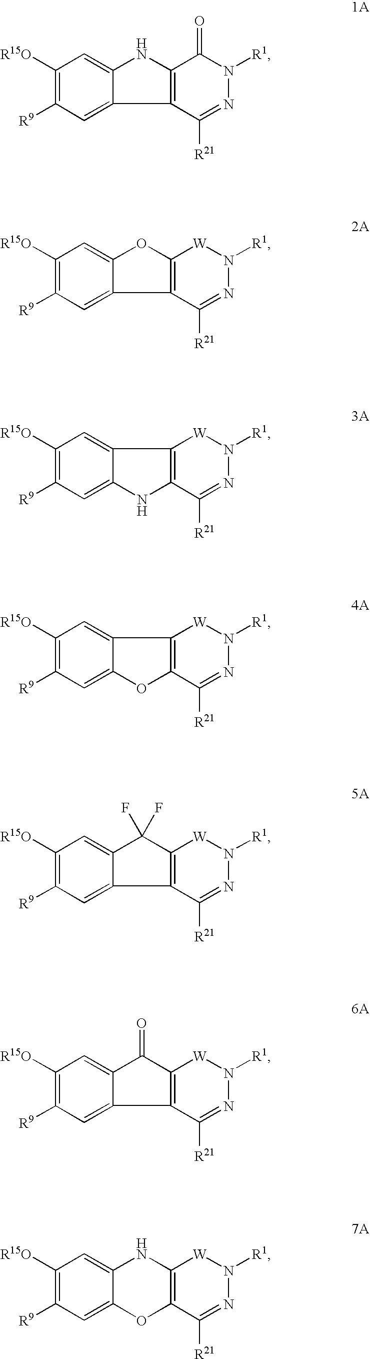

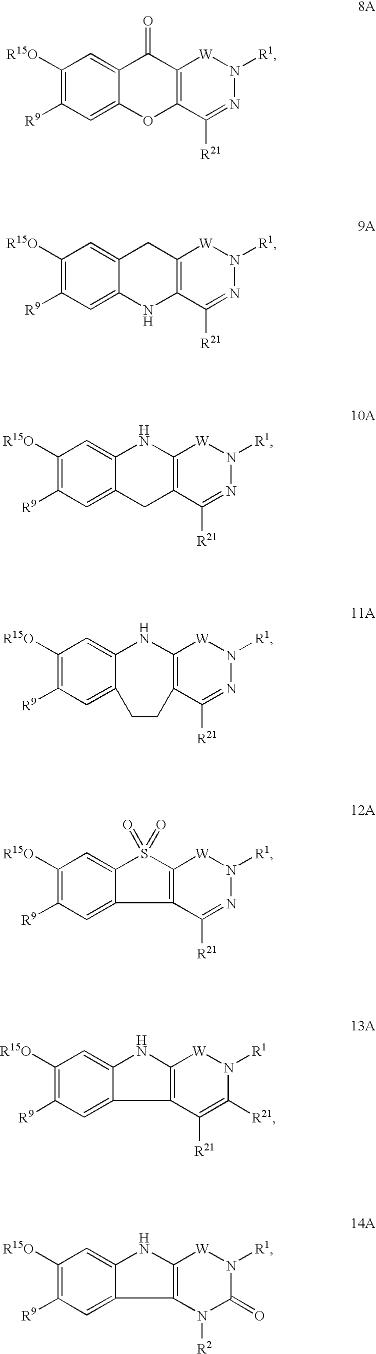

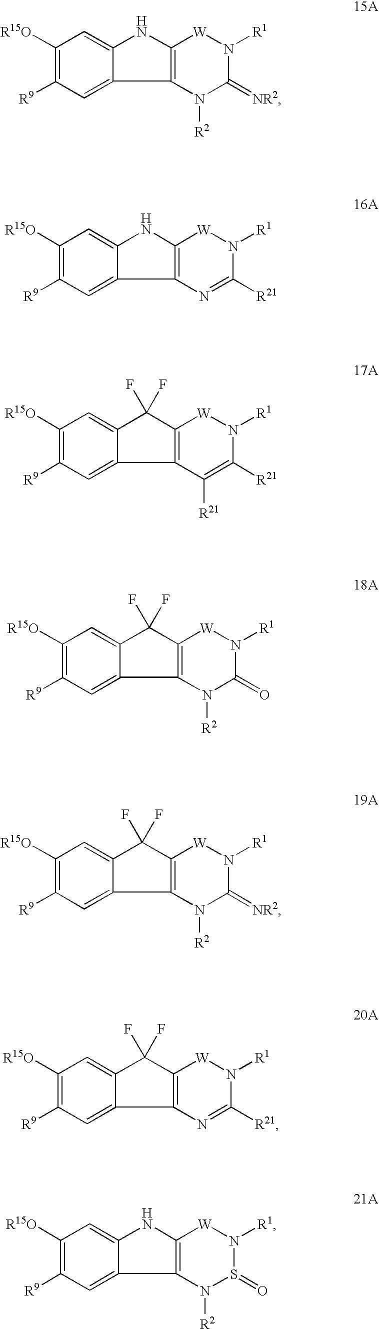

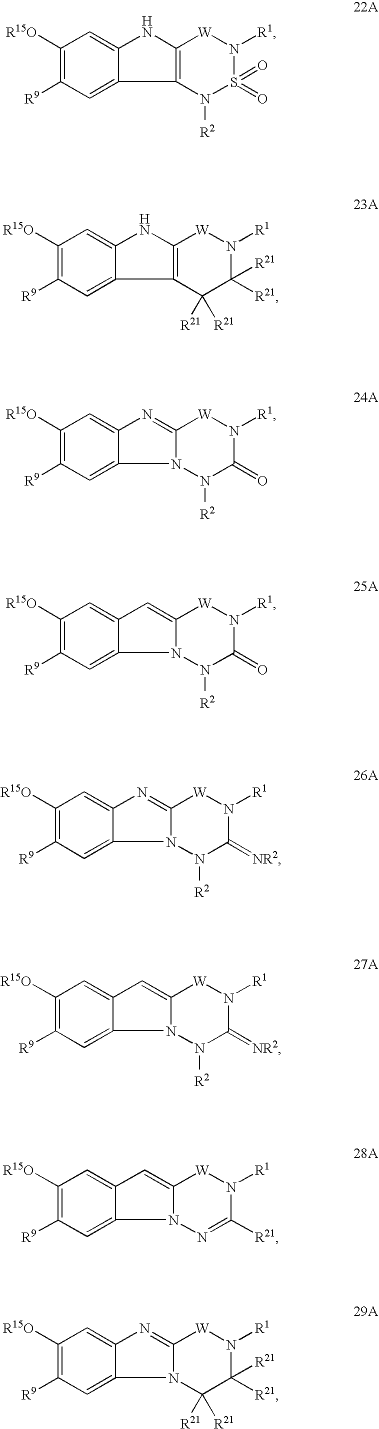

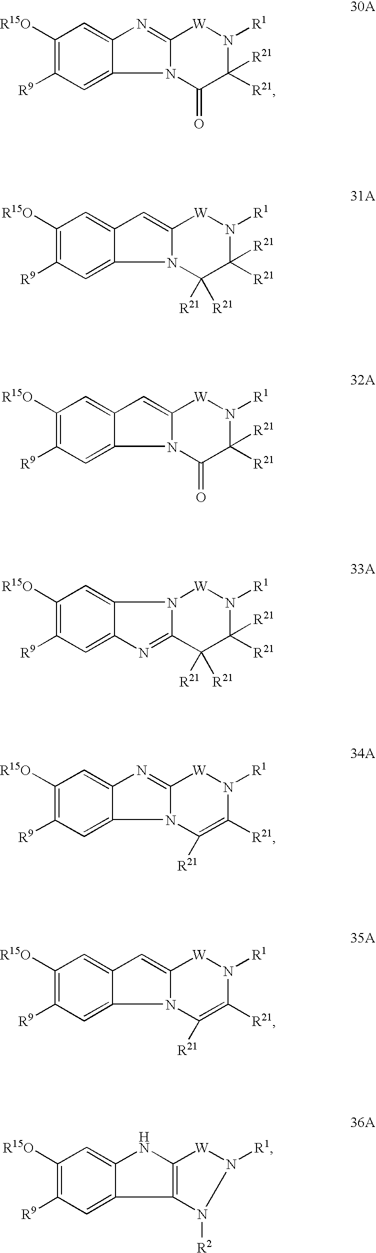

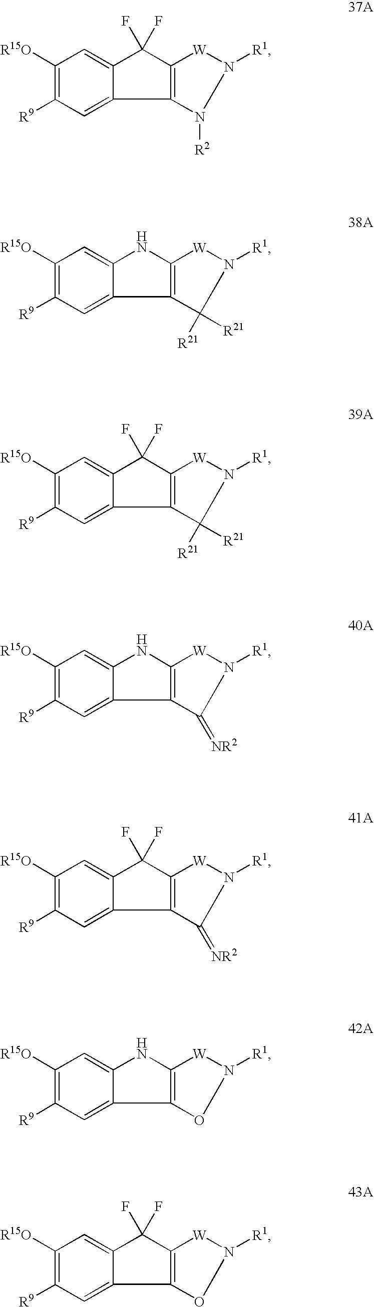

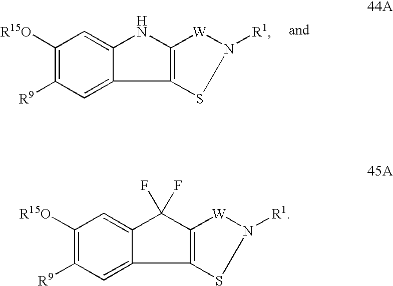

14. The compound of claim 1 selected from the group consisting of:

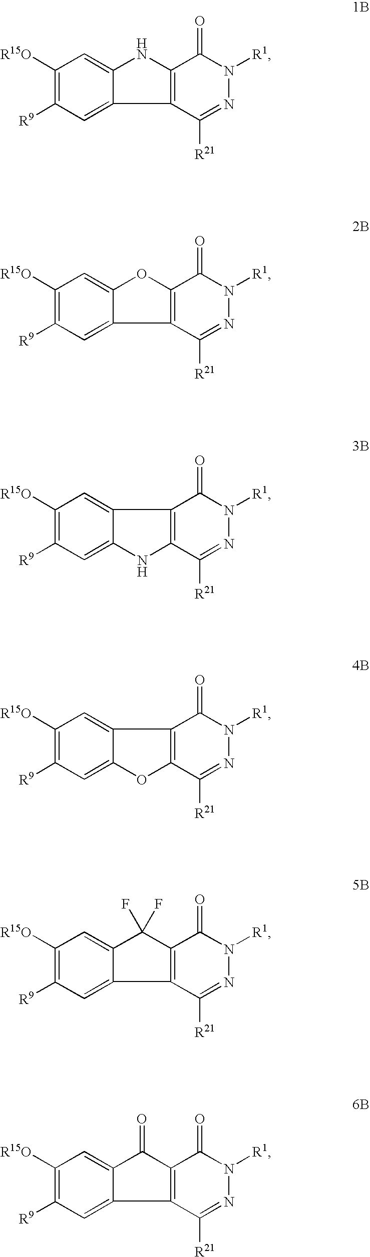

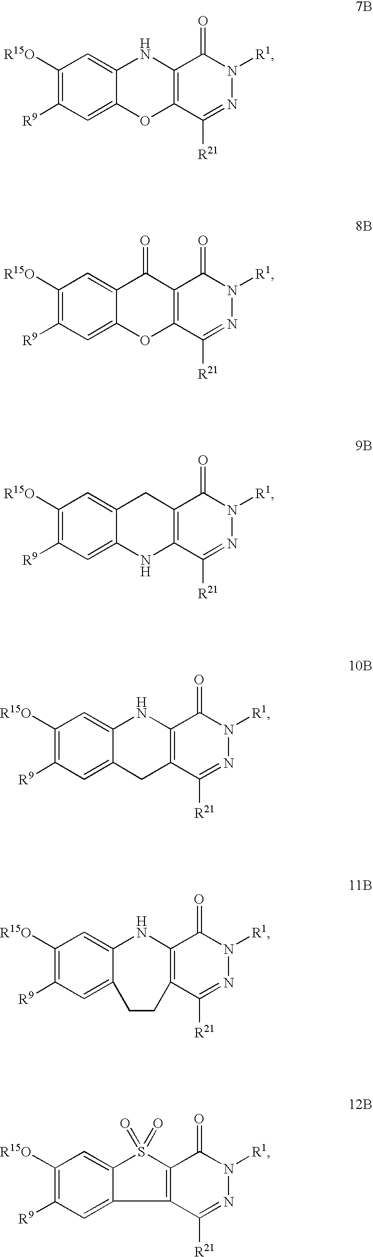

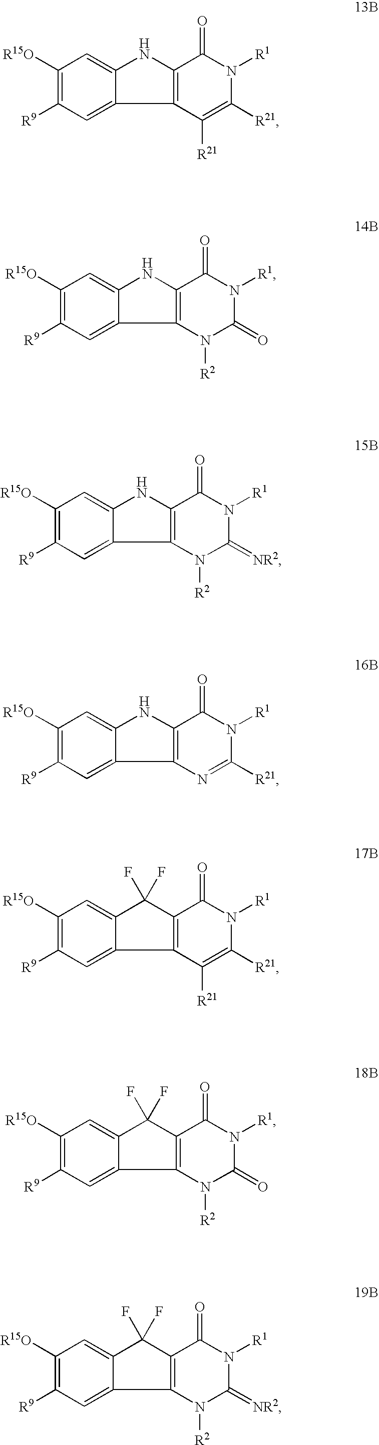

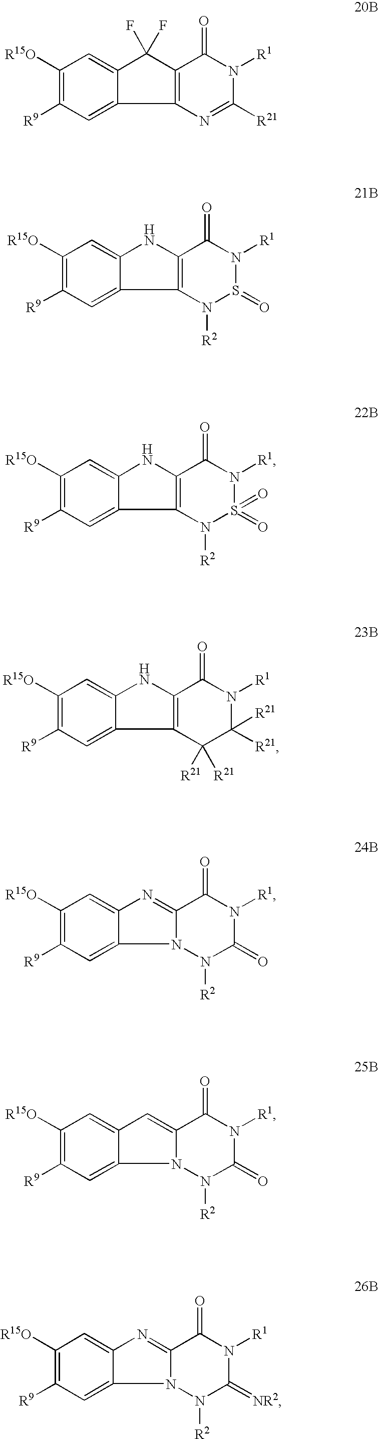

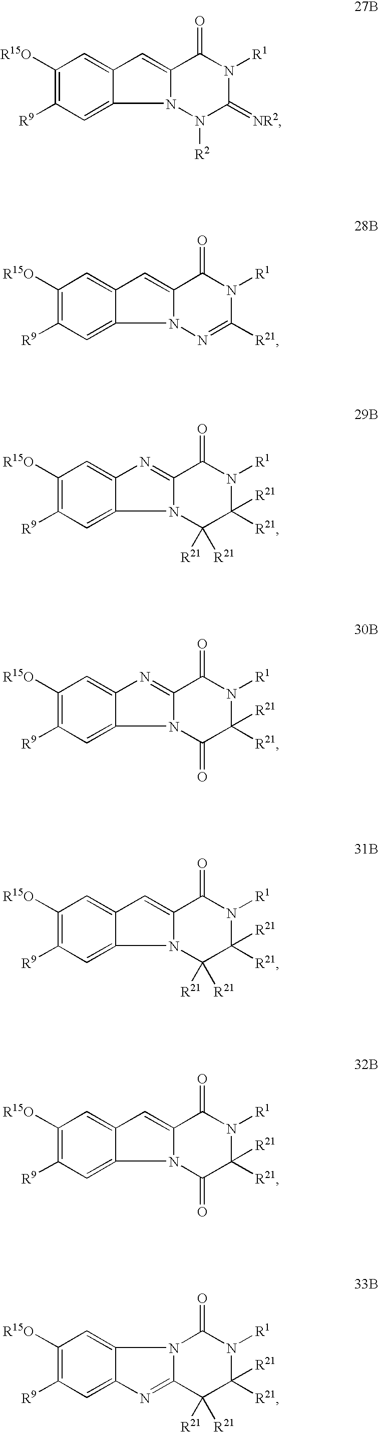

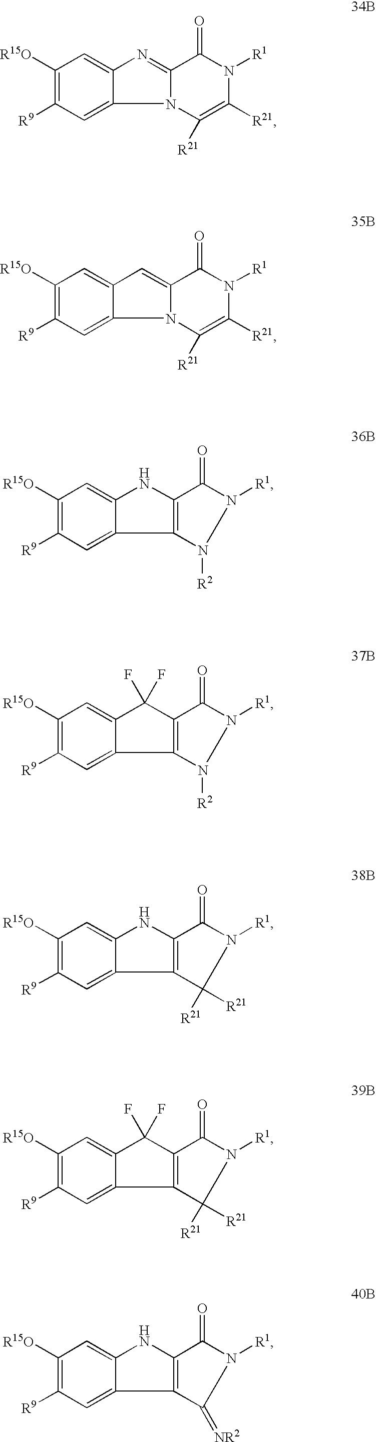

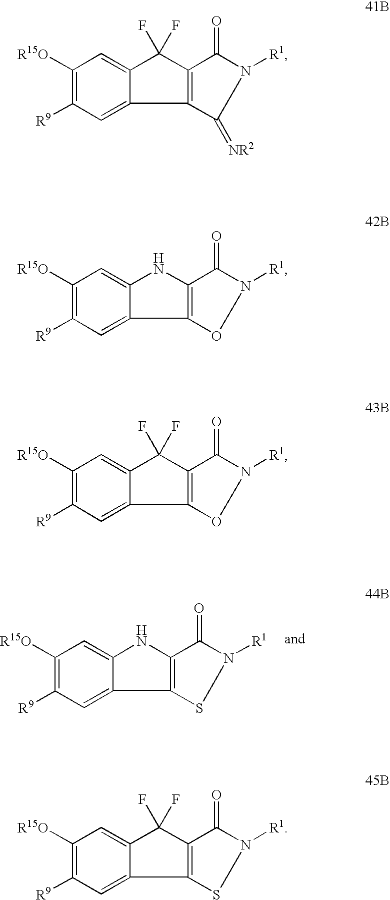

15. The compound of claim 1 selected from the group consisting of:

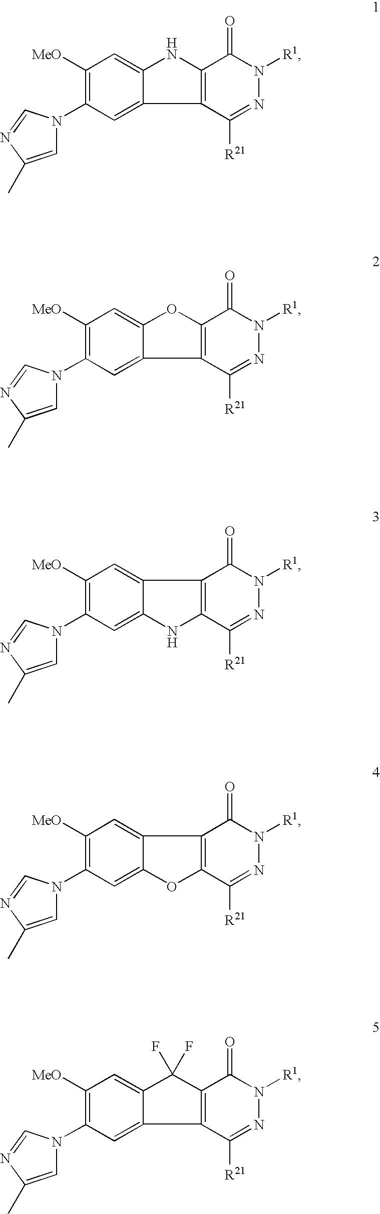

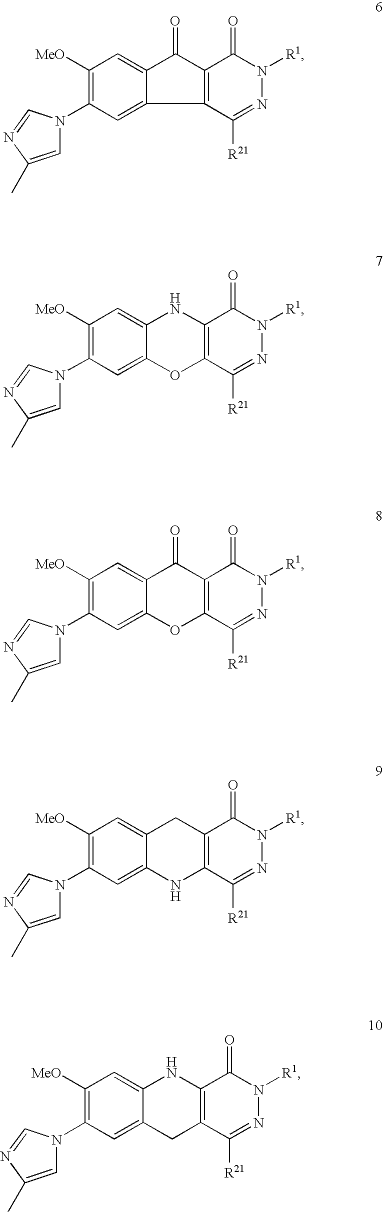

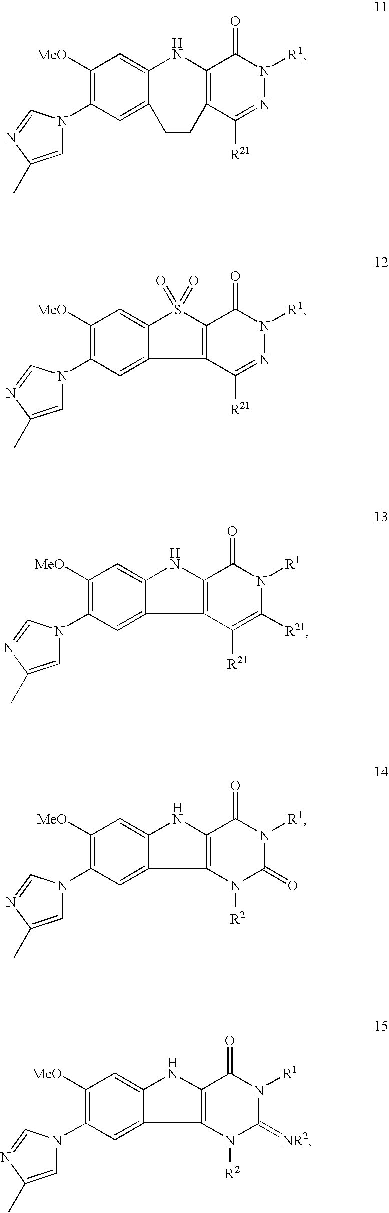

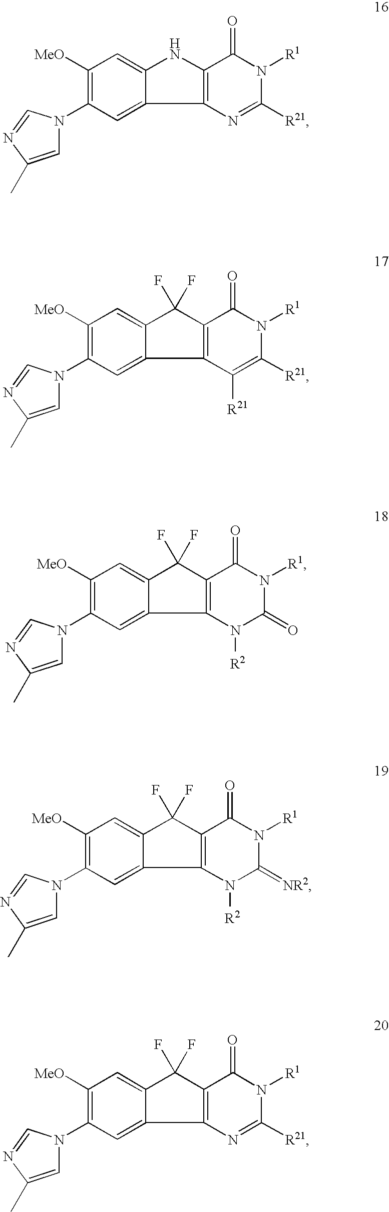

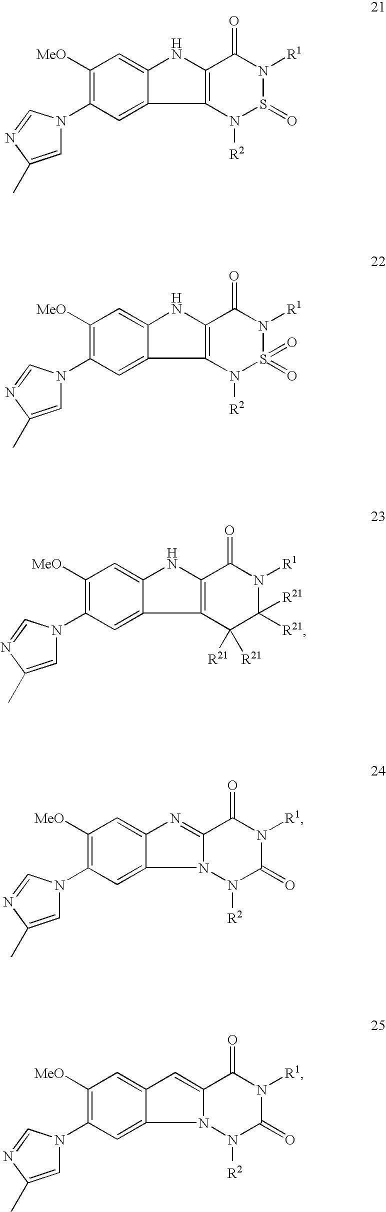

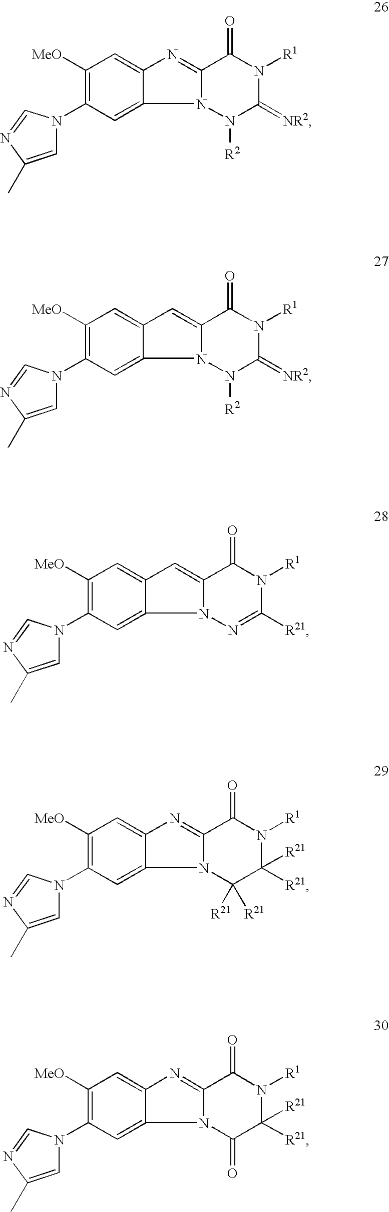

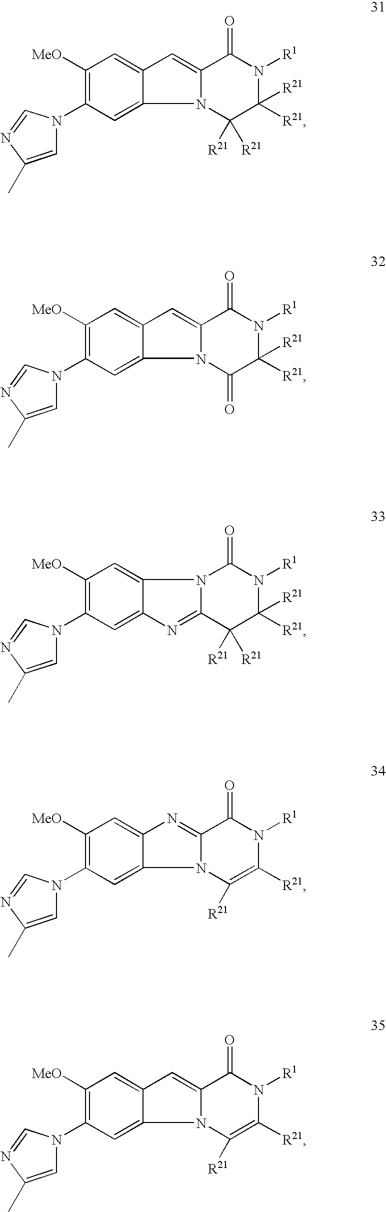

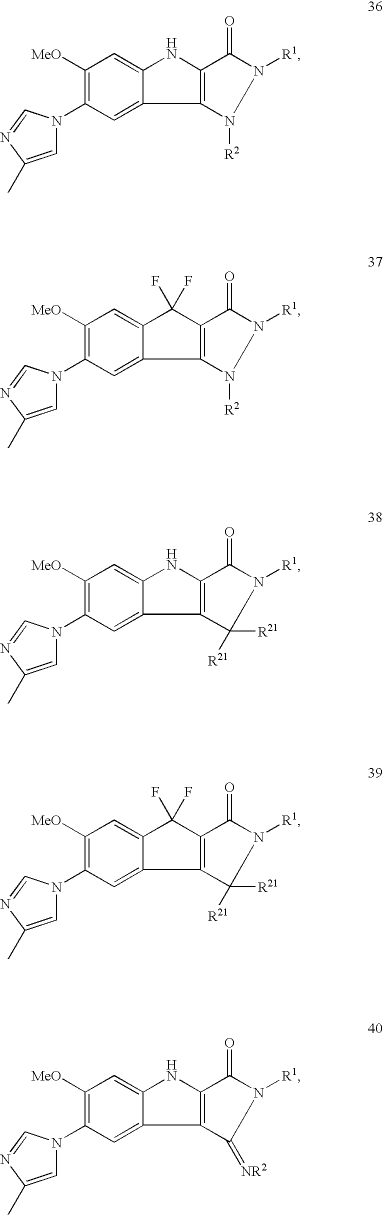

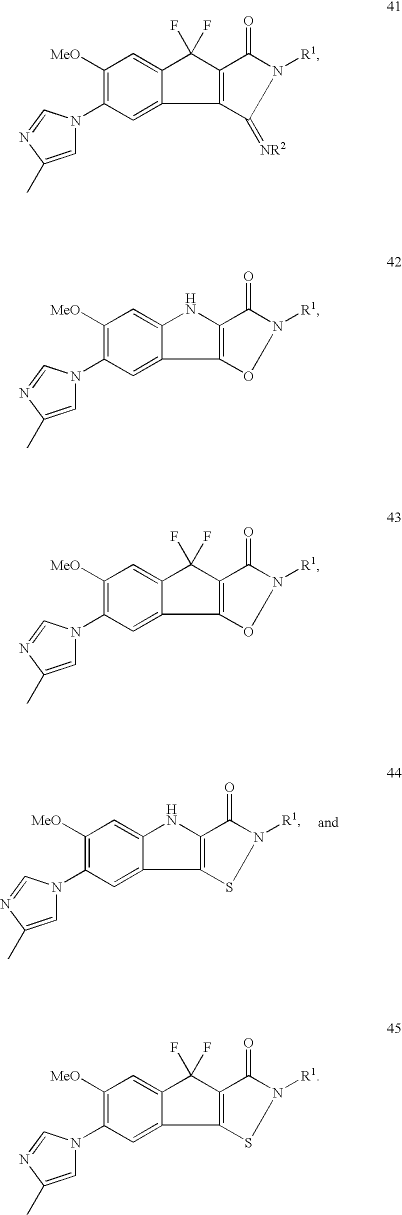

16. The compound of claim 1 selected from the group consisting of: compounds A1, A2, and A3.

17. A pharmaceutical composition comprising:
(a) a therapeutically effective amount of at least one compound of claim 1, or a pharmaceutically acceptable salt, solvate, ester or prodrug thereof, and at least one pharmaceutically acceptable carrier; or
(b) a therapeutically effective amount of at least one compound of claim 1, or a pharmaceutically acceptable salt, solvate, ester or prodrug thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

18. A method of treating a central nervous system disorder comprising:
(a) administering a therapeutically effective amount of at least one compound of claim 1 to a patient in need of such treatment; or
(a) administering a therapeutically effective amount of a pharmaceutical composition comprising a therapeutically effective amount of at least one compound of claim 1, or a pharmaceutically acceptable salt, solvate, ester or prodrug thereof, and at least one pharmaceutically acceptable carrier; or
(b) administering a therapeutically effective amount of a pharmaceutical composition comprising a therapeutically effective amount of at least one compound of claim 1, or a pharmaceutically acceptable salt, solvate, ester or prodrug thereof, and at least one pharmaceutically acceptable carrier, and a therapeutically effective amount of one or more compounds selected from the group consisting of cholinesterase inhibitors, Aβ antibody inhibitors, gamma secretase inhibitors and beta secretase inhibitors.

19. A method of treating Alzheimers disease comprising:
(a) administering a therapeutically effective amount of at least one compound of claim 1 to a patient in need of such treatment; or
(b) administering a therapeutically effective amount of at least one compound of claim 1, in combination with a therapeutically effective amount of a BACE inhibitor, to a patient in need of such treatment.

20. A method of treating Alzheimers disease comprising
(a) administering a therapeutically effective amount of at least one compound of claim 15 to a patient in need of such treatment; or
(b) administering a therapeutically effective amount of at least one compound of claim 15, in combination with a therapeutically effective amount of a BACE inhibitor, to a patient in need of such treatment.

21. A method of treating Downs syndrome comprising administering a therapeutically effective amount of at least one compound of claim 1 to a patient in need of such treatment.

22. A method of:
(a) modulating gamma secretase activity comprising administering an effective amount of a compound of claim 1 to a patient in need of such treatment; or
(b) inhibiting the deposition of beta amyloid protein comprising administering an effective amount of a compound of claim 1 to a patient in need of such treatment; or
(c) treating one or more neurodegenerative disease comprising administering an effective amount of a compound of claim 1 to a patient in need of such treatment.

23. The compound of claim 1 wherein R¹ is: 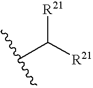
wherein one R²¹ is an unsubstituted or substituted alkyl group, and the other R²¹ is an unsubstituted or substituted arylgroup.

24. The compound of claim 22 wherein R¹ is: 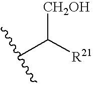
and R²¹ is unsubstituted aryl or aryl substituted with one or more independently selected R²² groups.
